SEQUENCE LISTING (126 sequences)

SEQ ID NO: 1 - protein, 121 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 2 - protein, 101 aa, Homo sapiens
QNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKD

SEQ ID NO: 3 - protein, 44 aa, Homo sapiens
PCGPCSERRKHLFVQDPQTCKCSCKNTDSRCKARQLELNERTCR

SEQ ID NO: 4 - DNA, 366 bp, Homo sapiens
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggtga

SEQ ID NO: 5 - protein, 14 aa, Artificial sequence
  misc_feature ()..(): Synthetic
PXCVXXXRCXGCCN

SEQ ID NO: 6 - protein, 25 aa, Homo sapiens
KKSVRGKGKGQKRKRKKSRYKSWSV

SEQ ID NO: 7 - protein, 6 aa, Homo sapiens
ARQEKC

SEQ ID NO: 8 - protein, 11 aa, Homo sapiens
ARQEKCDKPRR

SEQ ID NO: 9 - protein, 8 aa, Artificial sequence
  misc_feature ()..(): Synthetic
YVGARCCL

SEQ ID NO: 10 - protein, 8 aa, Artificial sequence
  misc_feature ()..(): Synthetic
MPWSLPGP

SEQ ID NO: 11 - protein, 16 aa, Homo sapiens
YVGARCCLMPWSLPGP

SEQ ID NO: 12 - protein, 23 aa, Homo sapiens
KSVRGKGKGQKRKRKKSRYKSWS

SEQ ID NO: 13 - protein, 11 aa, Artificial sequence
  misc_feature ()..(): Synthetic
CXXXRDGXXXC

SEQ ID NO: 14 - protein, 45 aa, Artificial sequence
  misc_feature ()..(): Synthetic
CXXXXXXXXXXXXCXCXXXXXXCXXXXXXCXXXXXXXXXXXXXXC

SEQ ID NO: 15 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 16 - DNA, 3041 bp, Homo sapiens
gaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggcgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaaccaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggatatatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttcatttaattgtttcagaataggataattaggtattcgaattttgtctttaaaattcatgtggtttctatgcaaagttcttcatatcatcacaacattatttgatttaaataaaattgaaagt

SEQ ID NO: 17 - protein, 192 aa, Homo sapiens
MPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 18 - protein, 235 aa, Homo sapiens
HDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFR

SEQ ID NO: 19 - protein, 49 aa, Homo sapiens
MTVFLSFAFLAAILTHIGCSNQRRSPENSGRRYNRIQHGQCAYTFILPE

SEQ ID NO: 20 - protein, 184 aa, Homo sapiens
HRLPRDCQELFQVGERQSGLFEIQPQGSPPFLVNCKMTSDGGWTVIQRRHDGSVDFNRPWEAYKAGFGDPHGEFWLGLEKVHSIMGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFR

SEQ ID NO: 21 - protein, 221 aa, Homo sapiens
EEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 22 - protein, 219 aa, Homo sapiens
KMGPKGEPGPRNCRELLSQGATLSGWYHLCLPEGRALPVFCDMDTEGGGWLVFQRRQDGSVDFFRSWSSYRAGFGNQESEFWLGNENLHQLTLQGNWELRVELEDFNGNRTFAHYATFRLLGEVDHYQLALGKFSEGTAGDSLSLHSGRPFTTYDADHDSSNSNCAVIVHGAWWYASCYRSNLNGRYAVSEAAAHKYGIDWASGRGVGHPYRRVRMMLR

SEQ ID NO: 23 - protein, 217 aa, Homo sapiens
DCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 24 - protein, 219 aa, Homo sapiens
KPVGPWQDCAEARQAGHEQSGVYELRVGRHVVSVWCEQQLEGGGWTVIQRRQDGSVNFFTTWQHYKAGFGRPDGEYWLGLEPVYQLTSRGDHELLVLLEDWGGRGARAHYDGFSLEPESDHYRLRLGQYHGDAGDSLSWHNDKPFSTVDRDRDSYSGNCALYQRGGWWYHACAHSNLNGVWHHGGHYRSRYQDGVYWAEFRGGAYSLRKAAMLIRPLKL

SEQ ID NO: 25 - protein, 215 aa, Homo sapiens
LPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQ

SEQ ID NO: 26 - protein, 222 aa, Artificial sequence
  misc_feature ()..(): Source not known
PRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 27 - protein, 222 aa, Artificial sequence
  misc_feature ()..(): Source not known
HDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 28 - protein, 214 aa, Mus musculus
RDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQP

SEQ ID NO: 29 - protein, 216 aa, Homo sapiens
FQDCAEIKRSGVNTSGVYTIYETNMTKPLKVFCDMETDGGGWTLIQHREDGSVNFQRTWEEYKEGFGNVAREHWLGNEAVHRLTSRTAYLLRVELHDWEGRQTSIQYENFQLGSERQRYSLSVNDSSSSAGRKNSLAPQGTKFSTKDMDNDNCMCKCAQMLSGGWWFDACGLSNLNGIYYSVHQHLHKINGIRWHYFRGPSYSLHGTRMMLRPMGA

SEQ ID NO: 30 - protein, 216 aa, Homo sapiens
FQDCAEIQRSGASASGVYTIQVSNATKPRKVFCDLQSSGGRWTLIQRRENGTVNFQRNWKDYKQGFGDPAGEHWLGNEVVHQLTRRAAYSLRVELQDWEGHEAYAQYEHFHLGSENQLYRLSVVGYSGSAGRQSSLVLQNTSFSTLDSDNDHCLCKCAQVMSGGWWFDACGLSNLNGVYYHAPDNKYKMDGIRWHYFKGPSYSLRASRMMIRPLDI

SEQ ID NO: 31 - protein, 224 aa, Homo sapiens
KPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 32 - protein, 220 aa, Homo sapiens
INEGPFKDCQQAKEAGHSVSGIYMIKPENSNGPMQLWCENSLDPGGWTVIQKRTDGSVNFFRNWENYKKGFGNIDGEYWLGLENIYMLSNQDNYKLLIELEDWSDKKVYAEYSSFRLEPESEFYRLRLGTYQGNAGDSMMWHNGKQFTTLDRDKDMYAGNCAHFHKGGWWYNACAHSNLNGVWYRGGHYRSKHQDGIFWAEYRGGSYSLRAVQMMIKPID

SEQ ID NO: 33 - protein, 136 aa, Homo sapiens
GKKEKPEKKVKKSDCGEWQWSVCVPTSGDCGLGTREGTRTGAECKQTMKTQRCKIPCNWKKQFGAECKYQFQAWGECDLNTALKTRTGSLKRALHNAECQKTVTISKPCGKLTKPKPQAESKKKKKEGKKQEKMLD

SEQ ID NO: 34 - protein, 121 aa, Homo sapiens
KKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 35 - protein, 43 aa, Homo sapiens
CKYQFQAWGECDLNTALKTRTGSLKRALHNAECQKTVTISKPC

SEQ ID NO: 36 - protein, 54 aa, Homo sapiens
AECKYQFQAWGECDLNTALKTRTGSLKRALHNAECQKTVTISKPCGKLTKPKPQ

SEQ ID NO: 37 - protein, 72 aa, Homo sapiens
AECKYQFQAWGECDLNTALKTRTGSLKRALHNAECQKTVTISKPCGKLTKPKPQAESKKKKKEGKKQEKMLD

SEQ ID NO: 38 - protein, 80 aa, Homo sapiens
CGEWTWGPCIPNSKDCGLGTREGTCKQETRKLKCKIPCNWKKQFGADCKYKFESWGECDANTGLKTRSGTLKKALYNADC

SEQ ID NO: 39 - protein, 21 aa, Homo sapiens
GKKEKPEKKVKKSDCGEWQWS

SEQ ID NO: 40 - protein, 16 aa, Homo sapiens
SKKKKKEGKKQEKMLD

SEQ ID NO: 41 - protein, 61 aa, Homo sapiens
DCKYKFENWGACDGGTGTKVRQGTLKKARYNAQCQETIRVTKPCTPKTKAKAKAKKGKGKD

SEQ ID NO: 42 - protein, 42 aa, Homo sapiens
KYKFENWGACDGGTGTKVRQGTLKKARYNAQCQETIRVTKPC

SEQ ID NO: 43 - protein, 32 aa, Homo sapiens
MQAQQYQQQRRKFAAAFLAFIFILAAVDTAEA

SEQ ID NO: 44 - protein, 20 aa, Homo sapiens
MQHRGFLLLTLLALLALTSA

SEQ ID NO: 45 - protein, 139 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSS

SEQ ID NO: 46 - protein, 15 aa, Homo sapiens
MAEGEITTFTALTEK

SEQ ID NO: 47 - protein, 8 aa, Homo sapiens
KKNGSCKR

SEQ ID NO: 48 - protein, 13 aa, Artificial sequence
  misc_feature ()..(): Synthetic
RLYCXLXXXPDGR

SEQ ID NO: 49 - protein, 4 aa, Homo sapiens
ISSK

SEQ ID NO: 50 - protein, 5 aa, Homo sapiens
KKPKL

SEQ ID NO: 51 - protein, 535 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 52 - protein, 22 aa, Homo sapiens
MLGPCMLLLLLLLGLRLQLSLG

SEQ ID NO: 53 - protein, 29 aa, Homo sapiens
AAHPGRSVVPALLPLLAGTLLLLETATAP

SEQ ID NO: 54 - protein, 108 aa, Homo sapiens
GMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTR

SEQ ID NO: 55 - protein, 20 aa, Artificial sequence
  misc_feature ()..(): Synthetic
AQVPDSAXTATAYLCGVKAN

SEQ ID NO: 56 - protein, 86 aa, Artificial sequence
  misc_feature ()..(): Synthetic
TNVAKNXIMFLGDGMGVSTVTAARILKGQXHHXXGXETXLXMDXFPXVALSKTYNXXAQVPDSAXTATAYLCGVKANXXTXGXSAA

SEQ ID NO: 57 - protein, 53 aa, Artificial sequence
  misc_feature ()..(): Synthetic
EDTLTXVTADHSHVFXFGGYTXRGNSIFGLAPMXXDTDKKXXTAILYGNGPGY

SEQ ID NO: 58 - protein, 22 aa, Homo sapiens
VVPALLPLLAGTLLLLETATAP

SEQ ID NO: 59 - protein, 154 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGCDKPRR

SEQ ID NO: 60 - protein, 162 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKCDKPRR

SEQ ID NO: 61 - protein, 150 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKKKCDKPRR

SEQ ID NO: 62 - protein, 154 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKKKKKKKCDKPRR

SEQ ID NO: 63 - protein, 7 aa, Artificial sequence
  misc_feature ()..(): Synthetic
GGGGSSS

SEQ ID NO: 64 - protein, 4 aa, Artificial sequence
  misc_feature ()..(): Synthetic
IEGR

SEQ ID NO: 65 - protein, 9 aa, Artificial sequence
  misc_feature ()..(): Synthetic
PGISGGGGG

SEQ ID NO: 66 - protein, 15 aa, Artificial sequence
  misc_feature ()..(): Synthetic
GGGGSGGGGSGGGGS

SEQ ID NO: 67 - protein, 13 aa, Artificial sequence
  misc_feature ()..(): Synthetic
EGKSSGSGSEKEF

SEQ ID NO: 68 - protein, 26 aa, Homo sapiens
MNFLLSWVHWSLALLLVLHHAKWSQA

SEQ ID NO: 69 - DNA, 33 bp, Artificial sequence
  misc_feature ()..(): Synthetic
cgcggatccaccatgaactttctgctgtcttgg

SEQ ID NO: 70 - DNA, 39 bp, Artificial sequence
  misc_feature ()..(): Synthetic
ctaaatggtttctcttcctccccgcctcggcttgtcaca

SEQ ID NO: 71 - DNA, 39 bp, Artificial sequence
  misc_feature ()..(): Synthetic
tgtgacaagcctgaggcgggaggaagagaaaccatttag

SEQ ID NO: 72 - DNA, 28 bp, Artificial sequence
  misc_feature ()..(): Synthetic
cgcggatcctcaaaaatctaaaggtcga

SEQ ID NO: 73 - DNA, 1107 bp, Artificial sequence
  misc_feature ()..(): Synthetic
gctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccaatgaactttctgctgtcttgggtgcattggagccttgccttgctgctctacctccaccatgccaagtggtcccagtcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcgggaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 74 - protein, 368 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 75 - DNA, 39 bp, Artificial sequence
  misc_feature ()..(): Synthetic
tttgcactccgcgccaaattgccgcctcggcttgtcaca

SEQ ID NO: 76 - DNA, 39 bp, Artificial sequence
  misc_feature ()..(): Synthetic
tgtgacaagccgaggcggcaatttggcgcggagtgcaaa

SEQ ID NO: 77 - DNA, 28 bp, Artificial sequence
  misc_feature ()..(): Synthetic
cgcggatccttaatccagcatcttctcc

SEQ ID NO: 78 - DNA, 669 bp, Artificial sequence
  misc_feature ()..(): Synthetic
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggcaatttggcgcggagtgcaaataccagttccaggcctggggagaatgtgacctgaacacagccctgaagaccagaactggaagtctgaagcgagccctgcacaatgccgaatgccagaagactgtcaccatctccaagccctgtggcaaactgaccaagcccaaacctcaagcagaatctaagaagaagaaaaaggaaggcaagaaacaggagaagatgctggattaa

SEQ ID NO: 79 - protein, 222 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRQFGAECKYQFQAWGECDLNTALKTRTGSLKRALHNAECQKTVTISKPCGKLTKPKPQAESKKKKKEGKKQEKMLD

SEQ ID NO: 80 - DNA, 37 bp, Artificial sequence
  misc_feature ()..(): Synthetic
tgcagtcggctccaaactcccgcctcggcttgtcaca

SEQ ID NO: 81 - DNA, 37 bp, Artificial sequence
  misc_feature ()..(): Synthetic
tgtgacaagccgaggcgggagtttggagccgactgca

SEQ ID NO: 82 - DNA, 27 bp, Artificial sequence
  misc_feature ()..(): Synthetic
cgcggatccctagtcctttcccttccc

SEQ ID NO: 83 - DNA, 639 bp, Artificial sequence
  misc_feature ()..(): Synthetic
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcgggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactag

SEQ ID NO: 84 - protein, 212 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRREFGADCKYKFENWGACDGGTGTKVRQGTLKKARYNAQCQETIRVTKPCTPKTKAKAKAKKGKGKD

SEQ ID NO: 85 - DNA, 36 bp, Artificial sequence
  misc_feature ()..(): Synthetic
ccatgggcccgacggcttccgcctcggcttgtcaca

SEQ ID NO: 86 - DNA, 36 bp, Artificial sequence
  misc_feature ()..(): Synthetic
tgtgacaagccgaggcggaagccgtcgggcccatgg

SEQ ID NO: 87 - DNA, 28 bp, Artificial sequence
  misc_feature ()..(): Synthetic
cgcggatccttagtggaaggtgttgggg

SEQ ID NO: 88 - DNA, 1116 bp, Artificial sequence
  misc_feature ()..(): Synthetic
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggaagccgtcgggcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaagcaagggtttgggaacattgatggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaacggggtctggtaccgcgggggccattaccggagccgctaccaggacggagtctactgggctgagttccgaggaggctcttactcactcaagaaagtggtgatgatgatccgaccgaaccccaacaccttccactaa

SEQ ID NO: 89 - protein, 371 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRKPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 90 - DNA, 36 bp, Artificial sequence
  misc_feature ()..(): Synthetic
gaatggtccttcattgatccgcctcggcttgtcaca

SEQ ID NO: 91 - DNA, 36 bp, Artificial sequence
  misc_feature ()..(): Synthetic
tgtgacaagccgaggcggatcaatgaaggaccattc

SEQ ID NO: 92 - DNA, 29 bp, Artificial sequence
  misc_feature ()..(): Synthetic
cgcggatcctcagtcaataggcttgatca

SEQ ID NO: 93 - DNA, 1104 bp, Artificial sequence
  misc_feature ()..(): Synthetic
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggatcaatgaaggaccattcaaagactgtcagcaagcaaaagaagctgggcattcggtcagtgggatttatatgattaaacctgaaaacagcaatggaccaatgcagttatggtgtgaaaacagtttggaccctgggggttggactgttattcagaaaagaacagacggctctgtcaacttcttcagaaattgggaaaattataagaaagggtttggaaacattgacggagaatactggcttggactggaaaatatctatatgcttagcaatcaagataattacaagttattgattgaattagaagactggagtgataaaaaagtctatgcagaatacagcagctttcgtctggaacctgaaagtgaattctatagactgcgcctgggaacttaccagggaaatgcaggggattctatgatgtggcataatggtaaacaattcaccacactggacagagataaagatatgtatgcaggaaactgcgcccactttcataaaggaggctggtggtacaatgcctgtgcacattctaacctaaatggagtatggtacagaggaggccattacagaagcaagcaccaagatggaattttctgggccgaatacagaggcgggtcatactccttaagagcagttcagatgatgatcaagcctattgactga

SEQ ID NO: 94 - protein, 367 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRINEGPFKDCQQAKEAGHSVSGIYMIKPENSNGPMQLWCENSLDPGGWTVIQKRTDGSVNFFRNWENYKKGFGNIDGEYWLGLENIYMLSNQDNYKLLIELEDWSDKKVYAEYSSFRLEPESEFYRLRLGTYQGNAGDSMMWHNGKQFTTLDRDKDMYAGNCAHFHKGGWWYNACAHSNLNGVWYRGGHYRSKHQDGIFWAEYRGGSYSLRAVQMMIKPID

SEQ ID NO: 95 - DNA, 1387 bp, Artificial sequence
  misc_feature ()..(): Synthetic
atgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaaggtagagaatatatcccaggacaacatgaagaaagaaatggtagagatacagcagaatgcagtacagaaccagacggctgtgatgatagaaatagggacaaacctgttgaaccaaacagcggagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcagaattccatcattgaagaactcgaaaaaaaaatagtgactgccacggtgaataattcagttcttcagaagcagcaacatgatctcatggagacagttaataacttactgactatgatgtccacatcaaacgcagctaaggaccccactgttgctaaagaagaacaaatcagcttcagagactgtgctgaagtattcaaatcaggacacaccacgaatggcatctacacgttaacattccctaattctacagaagagatcaaggcctactgtgacatggaagctggaggaggcgggtggacaattattcagcgacgtgaggatggcagcgttgcatttcagaggacttggaaagaatataaagtgggatttggtaacctctcagaaaaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggnnnnnnnnnnnnnnnnnnnggcaatgattttagcacaagggatggagccaccgncanatgtatttgcaaatgttcacaaatgctaacagnaggtnnnnnnnnnnnnnnnnnnnnnnnnnnnnnntactggaaaggctcaggctattcgctcaaggccacaaccatgatgatccgaccagcagatttc

SEQ ID NO: 96 - protein, 360 aa, Artificial sequence
  misc_feature ()..(): Synthetic
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENISQDNMKKEMVEIQQNAVQNQTAVMIEIGTNLLNQTAEQTRKLTDVEAQVSNATTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNCKXXXXVAKEEQISFRDCAEVFKSGHTTNGIYTLMWQIVFFTLSCDLVLAAAYNNFRKSMDSIGKKQYQVQHGSCSYTFLLPEMDNCRSSSSPY

SEQ ID NO: 97 - protein, 339 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 98 - DNA, 361 bp, Artificial sequence
  misc_feature ()..(): Synthetic
gtccaatgctgtgcagagggacgcgccgctcgaatacgatgactcggtgcagaggctgcaagtgctggagaacatcatggaaaacaacactcagtggctaatgaaggtagagaatatatcccaggacaacatgaagaaagaaatggtagagatacagcagaatgcagtacagaaccagacggctgtgatgatagaaatagggacaaacctgttgaaccaaacagcggagcaaacgcggaagttaactgatgtggaagcccaagtattaaatcagaccacgagacttgaacttcagctcttggaacactccctctcgacaaacaaattggaaaaacagattttggaccagaccagtgaaata

SEQ ID NO: 99 - protein, 123 aa, Artificial sequence
  misc_feature ()..(): Synthetic
VSNAVQRDAPLEYDDSVQRLQVLENIMENNTQWLMKLENISQDNMKKEMVEIQQNAVQNQTAVMIEIGTNLLNQTAEQTRKLTDVEAQVSNATTRLELQLLEHSLSTNKLEKQILDQTSEINK

SEQ ID NO: 100 - protein, 462 aa, Artificial sequence
  misc_feature ()..(): Synthetic
VSNAVQRDAPLEYDDSVQRLQVLENIMENNTQWLMKLENISQDNMKKEMVEIQQNAVQNQTAVMIEIGTNLLNQTAEQTRKLTDVEAQVSNATTRLELQLLEHSLSTNKLEKQILDQTSEINKMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 101 - protein, 224 aa, Homo sapiens; identical to SEQ ID NO: 31

SEQ ID NO: 102 - protein, 220 aa, Homo sapiens; identical to SEQ ID NO: 32

SEQ ID NO: 103 - protein, 371 aa, Artificial sequence; identical to SEQ ID NO: 89
  misc_feature ()..(): Synthetic

SEQ ID NO: 104 - protein, 367 aa, Artificial sequence; identical to SEQ ID NO: 94
  misc_feature ()..(): Synthetic

SEQ ID NO: 105 - protein, 53 aa, Homo sapiens
KLENYIQDNMKKEMVEIQQNAVQNQTAVMIEIGTNLLNQTAEQTRKLTDVEAQ

SEQ ID NO: 106 - protein, 105 aa, Homo sapiens
HGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHK

SEQ ID NO: 107 - protein, 192 aa, Homo sapiens
GPICVNTKGQDASTIKDMITRMDLENLKDVLSRQKREIDVLQLVVDVDGNIVNEVKLLRKESRNMNSRVTQLYMQLLHEIIRKRDNSLELSQLENKILNVTTEMLKMATRYRELEVKYASLTDLVNNQSVMITLLEEQCLRIFSRQDTHVSPPLVQVVPQHIPNSQQYTPGLLGGNEIQRDPGYPRDLMPPP

SEQ ID NO: 108 - protein, 196 aa, Homo sapiens
PYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMET

SEQ ID NO: 109 - protein, 105 aa, Homo sapiens; identical to SEQ ID NO: 106

SEQ ID NO: 110 - protein, 192 aa, Homo sapiens; identical to SEQ ID NO: 107

SEQ ID NO: 111 - protein, 135 aa, Homo sapiens
DASTIKDMITRMDLENLKDVLSRQKREIDVLQLVVDVDGNIVNEVKLLRKESRNMNSRVTQLYMQLLHEIIRKRDNSLELSQLENKILNVTTEMLKMATRYRELEVKYASLTDLVNNQSVMITLLEEQCLRIFSR

SEQ ID NO: 112 - protein, 101 aa, Homo sapiens
ELELLNNELLKQKRQIETLQQLVEVDGGIVSEVKLLRKESRNMNSRVTQLYMQLLHEIIRKRDNALELSQLENRILNQTADMLQLASKYKDLEHKYQHLAT

SEQ ID NO: 113 - protein, 493 aa, Homo sapiens
MRPLCVTCWWLGLLAAMGAVAGQEDGFEGTEEGSPREFIYLNRYKRAGESQDKCTYTFIVPQQRVTGAICVNSKEPEVLLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKYQHLATLAHNQSEIIAQLEEHCQRVPSARPVPQPPPAAPPRVYQPPTYNRIINQISTNEIQSDQNLKVLPPPLPTMPTLTSLPSSTDKPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 114 - protein, 54 aa, Homo sapiens
TNKLERQMLMQSRELQRLQGRNRALETRLQALEAQHQAQLNSLQEKREQLHSLL

SEQ ID NO: 115 - protein, 145 aa, Homo sapiens
TQQVKQLEQALQNNTQWLKKLERAIKTILRSKLEQVQQQMAQNQTAPMLELGTSLLNQTTAQIRKLTDMEAQLLNQTSRMDAQMPETFLSTNKLENQLLLQRQKLQQLQGQNSALEKRLQALETKQQEELASILSKKAKLLNTLS

SEQ ID NO: 116 - DNA, 465 bp, Homo sapiens
gcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaagcaagggtttgggaacattgacggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccacccaggacagagatcatgatgtctacacag

SEQ ID NO: 117 - DNA, 305 bp, Homo sapiens
ggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctkggtagcctacaaggcggtggttttgggggatccccacggcgagttctggcttgggtcttggagaaaggkgcatagcatcacggggggaccggaacagccgmctggccgtgcaamctgcggggactgggatgggcaaacgc

SEQ ID NO: 118 - DNA, 458 bp, Homo sapiens
  misc_feature (224)..(244): “n” may be any nucleotide
attataagctgcggctggggcgataccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaaccggggtctggtaccgcgggggccattaccggagccgctaccaggacggagngtactgggctgagttccgaggaggctcttactcactcaaggaaacgtggtgatgatgatccgaccgaaccccaacaccttccactaagccagctccccctcctgacctctccgtggccattgncaggangcccaccctggtcacgctggccacagcacanagaacaactcctcacnagttcatcctgaggctgggaggaccgggatgctggattctgttttnccgaagtcactg

SEQ ID NO: 119 - DNA, 173 bp, Artificial sequence
  misc_feature ()..(): Synthetic
tataagctgcggctggggcgataccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaaccg

SEQ ID NO: 120 - DNA, 638 bp, Artificial sequence
  misc_feature ()..(): Synthetic
gcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaagcaagggtttgggaacattgacggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccacccaggacagagatcatgatgtctacacagtataagctgcggctggggcgataccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaaccg

SEQ ID NO: 121 - DNA, 4045 bp, Artificial sequence
  misc_feature ()..(): Synthetic
gcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaagcaagggtttgggaacattgacggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccacccaggacagagatcatgatgtctacacagtataagctgcggctggggcgataccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaaccggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggcgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaaccaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggatatatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttcatttaattgtttcagaataggataattaggtattcgaattttgtctttaaaattcatgtggtttctatgcaaagttcttcatatcatcacaacattatttgatttaaataaaattgaaagtgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggtga

SEQ ID NO: 122 - protein, 280 aa, Artificial sequence
  misc_feature ()..(): Synthetic
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKVENISQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNAAKDPTVAKEEQIS

SEQ ID NO: 123 - protein, 221 aa, Homo sapiens
EEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 124 - DNA, 1506 bp, Artificial sequence
  misc_feature ()..(): Synthetic
atgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaaggtagagaatatatcccaggacaacatgaagaaagaaatggtagagatacagcagaatgcagtacagaaccagacggctgtgatgatagaaatagggacaaacctgttgaaccaaacagcggagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcagaattccatcattgaagaactcgaaaaaaaaatagtgactgccacggtgaataattcagttcttcagaagcagcaacatgatctcatggagacagttaataacttactgactatgatgtccacatcaaacgcagctaaggaccccactgttgctaaagaagaacaaatcagcgaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 125 - protein, 501 aa, Artificial sequence
  misc_feature ()..(): Synthetic
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKVENISQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNAAKDPTVAKEEQISEEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 126 - protein, 648 aa, Artificial sequence
  misc_feature ()..(): Synthetic
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRMWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKVENISQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNAAKDPTVAKEEQISEEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

(19) United States
(12) Patent Application Publication
(10) Pub. No.: US 2003/0027751 A1
(43) Pub. Date: Feb. 6, 2003

(54) VEGF fusion proteins

(75) Inventors: Imre Kovesdi, Rockville, MD (US); Paul D. Kessler, Frederick, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: GenVec, Inc., Gaithersburg, MD (US)

(21) Appl. No.: 09/832,355

(22) Filed: Apr. 10, 2001

(51) Int. Cl. A61K 38/18; C07K 14/515

(52) U.S. Cl. 514/12; 530/350

(57) ABSTRACT

The invention provides therapeutic fusion proteins which include a first peptide portion comprising a first non-heparin binding VEGF peptide portion and a second non-VEGF peptide portion covalently associated with the first peptide portion, which first and second peptide portions separately promote angiogenesis, bone growth, wound healing, or any combination thereof. Further provided are polynucleotides encoding such fusion proteins, vectors including such polynucleotides, methods of making such proteins, and methods of promoting angiogenesis, bone growth, and/or wound healing using such proteins, polynucleotides, and vectors.

### FIELD OF THE INVENTION

This invention pertains to fusion proteins, polynucleotides encoding such fusion proteins, and methods of producing and administering such fusion proteins and polynucleotides.

### BACKGROUND OF THE INVENTION

Several therapeutic proteins are known to be involved in angiogenesis, bone growth, and wound healing. An exemplary group of such proteins are the vascular endothelial growth factors (VEGFs).

Through recombinant DNA technology, scientists have been able to generate fusion proteins that contain the combined amino acid sequence of two or more proteins. Fusion proteins including a VEGF portion are known in the art. For example, U.S. Pat. No. 5,194,597 discloses fusion proteins, which include a platelet-derived growth factor (PDGF) portion and a vascular endothelial growth factor (VEGF) portion; International Patent Application WO 00/06195 discloses fusion proteins comprising specific VEGFs fused to a collagen-binding peptides, International Patent Application WO 00/37642 discloses fusion proteins including an angiopoietin portion fused to a VEGF portion; and U.S. Pat. No. 5,972,338 discloses fusion proteins including NL1, an angiopoietin homolog, and a VEGF. However, the VEGF fusion proteins of the '597 patent are believed to lack the ability to work on different aspects of a biological system (e.g., by targeting different receptors or promoting different aspects of a therapeutic biologic cascade), and those of the '642 application and '338 patent may have limited therapeutic potential due to poor in vivo half-life, limited in vivo mobility, undesired receptor interaction, interference with desired receptor binding, or combinations of such drawbacks, while the fusion proteins of the '195 application are limited in their range of therapeutic potential.

Accordingly, there remains a need for therapeutic fusion proteins which exhibit improved therapeutic potential over those presently known in the art. This invention provides such fusion proteins, polynucleotides that encode such fusion proteins, and methods of producing and administering such fusion proteins and polynucleotides. These and other advantages of the invention, as well as additional inventive features, will be apparent from the description of the invention provided herein.

### BRIEF SUMMARY OF THE INVENTION

The invention provides a fusion protein comprising a first non-heparin binding VEGF peptide portion and a second non-VEGF peptide portion covalently associated with the first peptide portion, which first and second peptide portions separately promote angiogenesis, bone growth, wound healing, or any combination thereof. The invention also provides polynucleotides encoding such fusion proteins, vectors including such polynucleotides, methods of making such proteins, and methods of promoting angiogenesis, bone growth, and/or wound healing using such proteins, polynucleotides, and vectors.

### DETAILED DESCRIPTION OF THE INVENTION

The invention provides a fusion protein including a first VEGF peptide portion (referred to herein as the “first” or “VEGF” peptide portion) and a second non-VEGF peptide portion (referred to herein as the “second” or “non-VEGF” peptide portion) covalently associated with the first peptide portion. The first and second peptide portions separately promote angiogenesis, bone growth, wound healing, or any combination thereof. The peptide portions can be any sequence of covalently-associated amino acid residues. Typically, the peptide portions will include an amino acid sequence of a naturally occurring protein or related amino acid sequence. A peptide portion can include an entire protein, e.g., a naturally occurring protein. The peptide portion can be any suitable size and consist of any suitable number of amino acid residues (e.g., 10, 20, 50, 75, 100, 400, 500, or more amino acid residues). Preferably, the peptide portion includes about 10-700 amino acid residues, more preferably about 20-600 amino acid residues, even more preferably about 50-500 amino acid residues (e.g., about 100-450 amino acid residues).

The first or VEGF peptide portion typically and preferably comprises a non-heparin-binding VEGF. As such, the term “VEGF peptide portion” or “first peptide portion” is directed to such peptides (although fusion proteins comprising heparin-binding VEGF peptide portions also are contemplated and separately discussed herein). The VEGF peptide portion can comprise any suitable non-heparin-binding VEGF. Preferably, the VEGF peptide portion includes a VEGF-A (VEGF-I). A particularly preferred non-heparin-binding VEGF-A isoform is human VEGF₁₂₁ (SEQ ID NO: 1) and homologs thereof (e.g., bovine or murine VEGF₁₂₀), which are described in, e.g., U.S. Pat. Nos. 5,219,739 and 5,194,596. Fragments of such VEGFs also can be used (e.g., a fragment comprising at least 65%, preferably at least 75%, and more preferably at least about 90% of VEGF₁₂₁). Typically and preferably, the non-heparin-binding VEGF portion will be a portion of a naturally occurring VEGF, e.g., human VEGF₁₂₁ (described generally in, e.g., Gitay-Goren et al., J. Biol. Chem., 271, 5519-23 (1996), and U.S. Pat. No. 5,219,739), VEGF-C, or VEGF-E (described generally in, e.g., Ogawa et al., J. Biol. Chem., 273(47), 31273-82 (1998), and Meyer et al., EMBO J., 18(2), 363-74 (1999)).

The VEGF peptide portion is not limited to naturally occurring non-heparin-binding VEGFs, but also can be a non-heparin-binding fragment of a naturally occurring heparin-binding VEGF (e.g., VEGF₁₁₀) (as described in, e.g., Keck et al., Arch. Biochem. Biophys., 344(1), 103-113 (1997)). Thus, for example, the VEGF peptide portion can include a non-heparin-binding fragment of a mammalian VEGF-B (VEGF-II) (e.g., VEGF-B₁₆₇ and VEGF-B₁₈₆) (described in, e.g., Grimmond et al., Genome Res., 6, 122-29 (1996), Olofsson et al., Proc. Natl. Acad. Sci. USA, 93, 2567-81 (1996), and U.S. Pat. No. 5,840,693, or a fragment of a modified VEGF-B (e.g., as described in International Patent Application WO 98/49300)), VEGF-C (described in, e.g., Joukov et al., EMBO J., 15, 290-98 (1996), and Lee et al., Proc. Natl. Acad. Sci. USA, 93, 1988-92 (1996)), VEGF-C (as described in e.g., Juokov et al., EMBO J., 16, 3898-11 (1997) and Lee et al., Proc. Natl. Acad. Sci. USA, 93, 1988-1992 (1996), VEGF-D (described in, e.g., Achen et al. Proc. Natl. Acad. Sci. USA, 95, 548-53 (1998) and International Patent Application WO 99/33485), Placenta Growth Factor (PlGF) (e.g., PlGF-129 or PlGF-1 50) (described in, e.g., Maglione et al., Proc. Natl. Acad. Sci. USA, 88, 9267-71 (1991)), mammalian VEGF-E (not to be confused with non-heparin-binding Orf virus VEGF-E, discussed further herein) (as described in, e.g., International Patent Application WO 99/47677), the “VEGF-3s” described in International Patent Application WO 00/09148, the VEGF-2s described in International Patent Application WO 95/24473, the VEGF-2 of U.S. Pat. No. 5,932,540, placenta growth factor (PlGF) (as described in, e.g., Achen et al., Int. J. Exp. Path., 79, 255-65 (1998) and references cited therein), GD-VEGF, or spinal cord derived growth factor (SCDGF) (as described in, e.g., Hanada et al., FEBS Lett., 475(2), 97-102 (2000)). A preferred fragment comprises the VEGF-A receptor binding domain (about residues 8-109 of VEGF₁₂₁, VEGF₁₆₅, VEGF₁₈₉, and VEGF₂₀₆) (SEQ ID NO: 2). Where the VEGF peptide portion comprises a non-heparin-binding fragment of a heparin-binding VEGF, or a truncated non-heparin-binding VEGF, it can be preferred that the VEGF peptide portion comprises the VEGF₁₁₀ sequence plus at least 5, more preferably at least 10 (but optionally more, e.g., 15, 20, or 25) amino acid sequences, which desirably correspond to or homologous with the 21 additional residues in VEGF₁₂₁.

Alternatively, the VEGF peptide portion can include a non-heparin-binding fragment of a non-mammalian VEGF, such as ORFV2-VEGF or OV-VEGF7 (as described in, e.g., Lyttle et al., J. Virol., 68, 84-92 (1991) and Ogawa et al., J. Biol. Chem., 273, 31273-82 (1998)). Where a non-heparin-binding fragment of an otherwise heparin-binding VEGF is used as the VEGF peptide portion, the VEGF peptide portion is preferably a fragment of a mammalian VEGF-A, such as VEGF₁₃₈, VEGF₁₄₅, VEGF₁₄₈, VEGF₁₆₂, VEGF₁₆₅, VEGF₁₈₂, VEGF₁₈₉, VEGF₂₀₆, P1GF-2, and variants thereof (as described in, e.g., Poltorak et al., J. Biol. Chem., 272, 7151-58 (1997), U.S. Pat. Nos. 6,057,428 and 6,013,780, and International Patent Applications WO 98/10071 and WO 99/40197). For example, the VEGF peptide portion can be a VEGF₁₈₉ or VEGF₁₆₅ fragment lacking about 25, preferably about 35, and more preferably about 40, of the amino acid residues located between positions 116 and 159 in these peptides (SEQ ID NO: 3). Other suitable fragments include modified wild-type VEGFs, such as the truncated VEGFs described in International Patent Application WO 98/49300.

Alternatively still, the VEGF peptide portion can include an amino acid sequence of a VEGF variant or homolog, which (1) exhibits high levels of amino acid sequence identity (either globally or locally) to a naturally occurring VEGF, (2) exhibits high levels of amino acid sequence homology to a naturally occurring VEGF, (3) exhibits a substantially similar hydrophilicity to a naturally occurring VEGF, (4) is encoded by a polynucleotide which hybridizes to a polynucleotide which encodes naturally occurring VEGF or a degenerate sequence thereof and which, when expressed, produces a non-heparin binding protein, or (5) meets any combination of (1)-(4). Preferably, the VEGF homolog exhibits high levels of sequence identity to a naturally occurring VEGF. VEGF homologs that do not exhibit high levels of identity to a naturally occurring VEGF preferably exhibit high levels of amino acid conservation and similar hydrophobicity to a naturally occurring VEGF. Such VEGF homolog peptide portions can be obtained in any suitable manner, including by synthetically preparing such homologs (e.g., through recombinant DNA technologies such as those further described herein) and identifying genes encoding naturally occurring VEGF homologs or orthologs, using techniques described herein and/or otherwise known in the art.

Preferably, the VEGF homolog peptide portion exhibits a significant level of identity to a naturally occurring VEGF, preferably a naturally non-heparin binding VEGF, and most preferably VEGF₁₂₁. The VEGF homolog peptide portion desirably exhibits at least about 50%, preferably at least about 75%, more preferably at least about 85%, and even more preferably at least about 90% amino acid global sequence identity (i.e., overall or total) to a naturally occurring VEGF (e.g., VEGF-E (as described in, e.g., Meyer et al., EMBO J., 18(2), 363-74 (1999)), PlGF-1, or VEGF121).

“Identity” with respect to amino acid or polynucleotide sequences refers to the percentage of residues or bases that are identical in the two sequences when the sequences are optimally aligned. If, in the optimal alignment, a position in a first sequence is occupied by the same amino acid residue or nucleotide as the corresponding position in the second sequence, the sequences exhibit identity with respect to that position. The level of identity between two sequences (or “percent sequence identity”) is measured as a ratio of the number of identical positions shared by the sequences with respect to the size of the sequences (i.e., percent sequence identity=(number of identical positions/total number of positions)×100).

The “optimal alignment” is the alignment which provides the highest identity between the aligned sequences. In obtaining the optimal alignment, gaps can be introduced, and some amount of non-identical sequences and/or ambiguous sequences can be ignored. Preferably, if a gap needs to be inserted into a first sequence to achieve the optimal alignment, the percent identity is calculated using only the residues that are paired with a corresponding amino acid residue (i.e., the calculation does not consider residues in the second sequences that are in the “gap” of the first sequence). However, it is often preferable that the introduction of gaps and/or the ignoring of non-homologous/ambiguous sequences is associated with a “gap penalty.”

A number of mathematical algorithms for rapidly obtaining the optimal alignment and calculating identity between two or more sequences are known and incorporated into a number of available software programs. Examples of such programs include the MATCH-BOX, MULTAIN, GCG, FASTA, and ROBUST programs for amino acid sequence analysis, and the SIM, GAP, NAP, LAP2, GAP2, and PIPMAKER programs for nucleotide sequences. Preferred software analysis programs for both amino acid and polynucleotide sequence analysis include the ALIGN, CLUSTAL-W (e.g., version 1.6 and later versions thereof), and BLAST programs (e.g., BLAST 2.1, BL2SEQ, and later versions thereof).

For amino acid sequence analysis, a weight matrix, such as the BLOSUM matrixes (e.g., the BLOSUM45, BLOSUM50, BLOSUM62, and BLOSUM80 matrixes), Gonnet matrixes (e.g., the Gonnet40, Gonnet80, Gonnet120, Gonnet160, Gonnet250, and Gonnet350 matrixes), or PAM matrixes (e.g., the PAM30, PAM70, PAM120, PAM160, PAM250, and PAM350 matrixes), are used in determining identity. BLOSUM matrixes are preferred. The BLOSUM50 and BLOSUM62 matrixes are typically most preferred. In the absence of availability to use such weight matrixes (e.g., in nucleic acid sequence analysis and with some amino acid analysis programs), a scoring pattern for residue/nucleotide matches and mismatches can be used (e.g., a +5 for a match −4 for a mismatch pattern).

The ALIGN program produces an optimal global alignment of the two chosen protein or nucleic acid sequences using a modification of the dynamic programming algorithm described by Myers and Miller, CABIOS, 4, 11-17 (1988). Preferably, if available, the ALIGN program is used with weighted end-gaps. If gap opening and gap extension penalties are available, they are preferably set between about −5 to −15 and 0 to −3, respectively, more preferably about −12 and −0.5 to −2, respectively, for amino acid sequence alignments, and −10 to −20 and −3 to −5, respectively, more preferably about −16 and −4, respectively, for nucleic acid sequence alignments. The ALIGN program and principles underlying it are further described in, e.g., Pearson et al., Proc. Natl. Acad. Sci. USA, 85, 2444-48 (1988), and Pearson et al., Methods Enzymol., 183, 63-98 (1990).

The BLAST programs provide analysis of at least two amino acid or nucleotide sequences, either by aligning a selected sequence against multiple sequences in a database (e.g., GenSeq), or, with BL2SEQ, between two selected sequences. BLAST programs are preferably modified by low complexity filtering programs such as the DUST or SEG programs, which are preferably integrated into the BLAST program operations (see, e.g., Wooton et al., Compu. Chem., 17, 149-63 (1993), Altschul et al., Nat. Genet., 6, 119-29 (1994), Hancock et al., Comput. Appl. Biosci. 10, 67-70 (1994), and Wootton et al., Meth. in Enzym., 266, 554-71 (1996)). If a lambda ratio is used, preferred settings for the ratio are between 0.75 and 0.95, more preferably between 0.8 and 0.9. If gap existence costs (or gap scores) are used, the gap existence cost is preferably set between about −5 and −15, more preferably about −10, and the per residue gap cost is preferably set between about 0 to −5, more preferably between 0 and −3 (e.g., −0.5). Similar gap parameters can be used with other programs as appropriate. The BLAST programs and principles underlying them are further described in, e.g., Altschul, et al., J. Mol. Biol., 215, 403-10 (1990), Karlin and Altschul, Proc. Natl. Acad. Sci. USA, 87, 2264-68 (1990) (as modified by Karlin and Altschul, Proc. Natl. Acad. Sci. USA, 90, 5873-77 (1993)), and Altschul et al., Nucl. Acids Res., 25, 3389-3402 (1997).

For multiple sequence analysis, the CULSTAL-W program can be used. The CLUSTAL-W program desirably is run using “dynamic” (versus “fast”) settings. Preferably, nucleotide sequences are compared using the BESTFIT matrix, whereas amino acid sequences are evaluated using a variable set of BLOSUM matrixes depending on the level of identity between the sequences (e.g., as used by the CLUSTAL-W version 1.6 program available through the San Diego Supercomputer Center (SDSC)). Preferably, the CLUSTAL-W settings are set to the SDSC CLUSTAL-W default settings (e.g., with respect to special hydrophilic gap penalties in amino acid sequence analysis). The CLUSTAL-W program and underlying principles of operation are further described in, e.g., Higgins et al., CABIOS, 8(2), 189-91 (1992), Thompson et al., Nucleic Acids Res., 22, 4673-80 (1994), and Jeanmougin et al., Trends Biochem. Sci., 23, 403-07 (1998).

Several commercially available software suites incorporate the ALIGN, BLAST, and CLUSTAL-W programs and similar functions, and may include significant improvements in settings and analysis. Examples of such programs include the GCG suite of programs and those available through DNASTAR, Inc. (Madison, Wis.). Particular preferred programs include the Lasergene and Protean programs sold by DNASTAR.

Because various algorithms, matrixes, and programs are commonly used to analyze sequences, “identity” is commonly understood in the art to represent a variable measurement. Accordingly, the identity between two sequences is preferably not limited to any exact measurement by a single technique, but, rather, is understood to represent an approximate range “about” a particular identity (e.g., +/−10%, more preferably +/−8%, and even more preferably +/−5% of the particular identity). Alternatively, an exact identity can be measured by using only one of the aforementioned programs, preferably one of the BLAST programs, as described herein.

The VEGF homolog peptide portion also can include or consist of a peptide which exhibits significant levels of local sequence identity to a naturally occurring, preferably naturally non-heparin-binding, VEGF, despite lacking an overall sequence identity at the above-described levels. For example, VEGF homolog peptide portions which exhibit at least about 70% identity, preferably at least about 80%, and more preferably at least about 90% identity, across a local alignment of at least about 65, preferably at least about 75, and more preferably at least about 90 amino acid residues, to a naturally occurring VEGF (e.g., VEGF₁₂₁) can be suitable.

Local sequence alignment can be determined using local sequence alignment software, e.g., the BLAST programs described above, the LFASTA program, or, more preferably, the LALIGN program. Preferably, the LALIGN program using a BLOSUM50 matrix analysis is used for amino acid sequence analysis, and a +5 match/−4 mismatch analysis is used for polynucleotide sequence analysis. Gap extension and opening penalties are preferably the same as those described above with respect to analysis with the ALIGN program. For LALIGN (or other program) analysis using k-tup value settings (also referred to as “k-tuple” or ktup values), a k-tup value of 0-3 for proteins, and 0-10 (e.g., about 6) for nucleotide sequences, is preferred.

The VEGF homolog peptide portion can alternatively include a peptide portion that exhibits high levels of homology to a naturally occurring, preferably naturally non-heparin binding, VEGF, despite lacking the above-described levels of global or local identity. For example, a VEGF homolog peptide portion which exhibits at least about 80%, preferably at least about 90%, and more preferably at least about 95% to a naturally occurring VEGF homology amino acid sequence, can be suitable, even though the homolog exhibits relatively low levels of identity (e.g., less than about 40% identity) to its wild-type VEGF homolog. “Homology” is a function of the number of corresponding conserved and identical amino acid residues in the optimal homology alignment. The “optimal homology alignment” is the alignment which provides the highest level of homology between two amino acid sequences, using the principles described above with respect to the “optimal alignment.” Conservative amino acid residue substitutions involve exchanging a member within one class of amino acid residues for a residue that belongs to the same class. VEGF homolog peptide portions containing conservative substitutions are expected to substantially retain the biological properties and functions associated with their wild-type counterpart. The classes of amino acids and the members of those classes are presented in Table 1.

TABLE 1
Amino Acid Residue Classes

| Amino Acid Class | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

Preferably, the highly hydrophilic VEGF homolog peptide portion or highly conserved VEGF homolog peptide portion exhibits high weight homology to a naturally occurring VEGF, most preferably VEGF₁₂₁. “High weight homology” means that at least about 40%, preferably at least about 60%, and more preferably at least about 70% of the non-identical amino acid residues are members of the same weight-based “weak conservation group” or “strong conservation group” as the corresponding amino acid residue in the wild-type VEGF. Strong group conservation is preferred. Weight-based conservation is determined on the basis of whether the non-identical corresponding amino acid is associated with a positive score on one of the weight-based matrices described herein (e.g., the BLOSUM50 matrix), typically and preferably the PAM250 matrix. Weight-based strong conservation groups include STA, NEQK, NHQK, NDEQ, QHRK, MILV, MILF, HY, and FYW. Weight-based weak conservation groups include CSA, ATV, SAG, STNK, STPA, SGND, SNDEQK, NDEQHK, NEQHRK, FVLIM, and HFY. The CLUSTAL W sequence analysis program provides analysis of weight based strong conservation and weak conservation groups in its output, and offers the preferred technique for determining weight-based conservation, preferably using the CLUSTAL W default settings used by the San Diego Supercomputer (SDSC).

Alternatively, the VEGF peptide portion can include a peptide exhibiting high levels of hydrophobicity/hydrophilicity conservation (“hydrophilicity”) to a naturally occurring, preferably naturally non-heparin binding, VEGF, optimally VEGF₁₂₁. Hydrophilicity can be determined using the Key & Doolittle index, the scores for each naturally occurring amino acid in the index being as follows: I (+4.5), V (+4.2), L (+3.8), F (+2.8), C (+2.5), M (+1.9); A (+1.8), G (−0.4), T (−0.7), S (−0.8), W (−0.9), Y (−1.3), P (−1.6), H (−3.2); E (−3.5), Q (−3.5), D (−3.5), N (−3.5), K (−3.9), and R (−4.5) (see, e.g., U.S. Pat. No. 4,554,101 for further discussion). The VEGF portion can include a peptide where at least 45%, preferably at least 60%, and more preferably at least 75% (e.g., at least 85%, 90%, or 95%) of the amino acid residues which differ from the naturally occurring VEGF exhibit less than a +/−2 change in hydrophilicity, more preferably less than a +/−1 change in hydrophilicity, and even more preferably less than a +/−0.5 change in hydrophilicity. Thus, the VEGF peptide portion preferably exhibits a total change in hydrophilicity of less than about 150, more preferably less than about 100, and even more preferably less than about 50 (e.g., less than about 30, 20, or 10). Examples of typical amino acid substitutions which retain similar or identical hydrophilicity include arginine-lysine substitutions, glutamate-aspartate substitutions, serine-threonine substitutions, glutamine-asparagine substitutions, and valine-leucine-isoleucine substitutions.

In yet another alternative, the non-heparin binding VEGF homolog peptide can include a peptide encoded by a polynucleotide that hybridizes to (1) the complement of a polynucleotide that, when expressed, results in a naturally occurring non-heparin-binding VEGF (e.g., a polynucleotide encoding human VEGF₁₂₁ (SEQ ID NO: 4)) or (2) a polynucleotide which would hybridize to the complement of such a sequence but for the degeneracy of the genetic code, under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5× Denhardt's solution, 10% dextran sulfate, and 20 mg/mL denatured sheared salmon sperm DNA, followed by washing the filters in 1×SSC at about 37-50° C., or substantially similar conditions, e.g., the moderately stringent conditions described in Sambrook et al., Molecular Cloning: A Laboratory Manual (Cold Spring Harbor Press 1989). High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0. 1% Ficoll/0.1% polyvinylpyrrolidone (PVP)/50 mM sodium phosphate buffer at pH 6.5 with 750 mM sodium chloride, 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at (i) 42° C. in 0.2×SSC, (ii) at 55° C. in 50% formamide and (iii) at 55° C. in 0.1×SSC (preferably in combination with EDTA). Additional details and explanation of stringency of hybridization reactions are provided in, e.g., Ausubel et al., Current Protocols in Molecular Biology (Wiley Interscience Publishers 1995).

The VEGF homolog peptide portion desirably retains identity to naturally occurring VEGFs at highly conserved residues. Conserved residues can be identified by using CLUSTAL-W or a similar program to identify positions where sequences are identical across many, most, or all of the members of a group of related proteins in the original alignment. Thus, for example, the VEGF peptide portion preferably retains the eight cysteine residues that are positionally conserved within the VEGF-A and PDGF protein families; preferably the VEGF peptide portion retains the cysteine knot structure formed by the non-dimer associated cysteines in this conserved domain, and more particularly the VEGF portion preferably comprises an amino acid sequence having the sequence pattern Pro Xaa Cys Val Xaa Xaa Xaa Arg Cys Xaa Gly Cys Cys Asn (SEQ ID NO: 5), where Xaa represents any amino acid residue, preferably a residue selected from one of the twenty naturally occurring amino acids. Desirably, the VEGF portion also or alternatively retains conserved residues in the kinase-insert domain-containing (KDR) receptor-binding domain of KDR-binding VEGFs, such as Arg₈₂, Lys₈₄, His₈₆, and/or, even more preferably, Asp₆₃, Glu₆₄, and Glu₆₇, and the hydrophobic residues within about 55 amino acid residues or less (e.g., 50 residues or less, 40 residues or less, or 30 residues or less) of Asp₆₃ (such sequences are in reference to the positions in the N-terminus of all VEGF-A isoforms, e.g., VEGF₁₂₁). Advantageously, the VEGF peptide portion retains the conserved residues/sequences necessary to induce authophosphorylation at human KDR receptor positions 1054 and 1059 (or their analogs in other species), which maximizes KDR kinase activity. Also desirably, the VEGF portion retains the VEGF glycosylation site at or near Asn₇₅ in wild type VEGF-As, or a functionally similar counterpart thereof.

The VEGF homolog peptide portion is desirably recognized by anti-VEGF antibodies, preferably human anti-VEGF antibodies, and desirably at least one monoclonal anti-VEGF antibody. Any suitable anti-VEGF antibody can be used. Examples of suitable antibodies are described in, e.g., Kim et al., Nature, 362, 841-44 (1993), Borgstrom et al., Cancer Res., 56(17), 4032-39 (1996), Presta et al., Cancer Res., 57(20), 4593-99 (1997), Wang et al., J. Cancer Res., Clin. Oncol., 124(11), 615-20 (1998), Asano et al. Jpn. J. Cancer Res., 90(1), 93-100 (1999), Mordenti et al., Toxicol. Pathol., 27(1):, 14-21 (1999), and Schlaeppi et al., J. Cancer Res., Clin. Oncol., 125(6), 336-42 (1999), as well as U.S. Pat. No. 5,219,739.

The VEGF homolog peptide portion preferably comprises a region of structural similarity to a non-heparin binding VEGF, preferably VEGF₁₂₁, or a non-heparin-binding VEGF fragment (e.g., VEGF₁₁₀). VEGF peptide portions comprising a portion exhibiting structural similarity to VEGF₁₂₁ (i.e., including the C-terminal domain thereof), or consisting essentially of such a structure, are particularly preferred. Structural similarity can be determined by any suitable technique, preferably using a suitable software program for making such assessments. Examples of such programs include the MAPS program and the TOP program (described in Lu, Protein Data Bank Quarterly Newsletter, #78, 10-11 (1996), and Lu, J. Appl. Cryst., 33, 176-183 (2000)). Using these programs the VEGF homolog peptide portion will desirably exhibit a low structural diversity, topological diversity (e.g., a topical diversity of less than about 20, preferably less than about 15, and more preferably less than about 10), or both. Alternatively, the homolog can be compared to the desired VEGF using the PROCHECK program (described in, e.g., Laskowski, J. Appl. Cryst., 26, 283-291 (1993)), the MODELLER program, or commercially available programs incorporating such features. Alternatively, a sequence comparison using a program such as the PredictProtein server (available at http://dodo.cpmc.columbia.edu/predictprotein/) should reveal similar structure for the VEGF homolog peptide portion and a wild-type non-heparin-binding VEGF, preferably VEGF₁₂₁.

The administration of the VEGF homolog peptide portion, or expression of the peptide portion from a polynucleotide, preferably induces the synthesis of plasminogen activator, plasminogen activator inhibitor type-1, interstitial collagenase, or a combination thereof.

Polynucleotides encoding VEGF homologs including sequences encoding VEGF homolog peptide portions can be identified in living systems through screening polynucleotide libraries (e.g., a genomic library, cDNA library, or sublibrary thereof). Such screening can be performed by any suitable technique, including, e.g., screening the libraries with polynucleotide probes under conditions wherein hybridization to VEGF homolog-encoding polynucleotides is likely to occur (e.g., under at least moderately stringent conditions). Such screening can be performed in a human DNA or cDNA library (e.g., to determine novel VEGF splice variants or homologs), or in a polynucleotide library obtained from other species, preferably other mammalian species (e.g., Pan troglodytes, Gorilla gorilla, Pongo pygmaeus, Hvlobates concolor, Macaca mulatta, Papio papio, Papio hamadrvns, Cercopithecus aethiops, Cebus capucinus, Aotus trivirgatus, Sanguinus oedipus, Microcebus murinus, Mus musculus, Rattus norvegicus, Cricetulus griseus, Felis catus, Mustela vison, Canisfamiliaris, Orystolagus cuniculus, Bos taurus, Ovis aries, Sus scrofa, and Equus caballus). Fluorescence in situ hybridization (FISH) of a cDNA clone to a metaphase chromosomal spread can be used to perform chromosomal screening for VEGF homolog-encoding genes. Pools of protein candidates can be similarly screened for VEGF homologs using standard biochemical and proteomics-related techniques (e.g., the yeast two hybrid system as described in, e.g., Mendelsohn and Brat, Curr. Opn. Biotech., 5, 482-86 (1994), and/or affinity chromatography (e.g., using the KDR receptor or portion thereof)). The VEGF peptide portion and second peptide portion can be derived from the same or different species (e.g., the fusion protein can include a bovine or murine VEGF peptide portion and a human derived second peptide portion).

Polynucleotides comprising sequences encoding novel VEGF homolog peptide portions (also referred to as VEGF variant-encoding polynucleotides) also can be synthesized through inducing mutations in known VEGF-encoding polynucleotides (e.g., a VEGF-E gene sequence or VEGF₁₂₁ gene sequence). For example, VEGF variant-encoding polynucleotides can be obtained through application of site-directed mutagenesis (as described in, e.g., Edelman et al., DNA, 2, 183 (1983), Zoller et al., Nucl. Acids Res., 10, 6487-5400 (1982), and Veira et al., Meth. Enzymol., 153, 3 (1987)), alanine scanning, or random mutagenesis, such as iterated random point mutagenesis induced by error-prone PCR, chemical mutagen exposure, or polynucleotide expression in mutator cells (see, e.g., Bomscheueret al., Biotechnol. Bioeng., 58, 554-59 (1998), Cadwell and Joyce, PCR Methods Appl., 3(6), S136-40 (1994), Kunkel et al., Methods Enzymol., 204, 125-39 (1991), Low et al., J. Mol. Biol., 260, 359-68 (1996), Taguchi et al., Appl. Environ. Microbiol., 64(2), 492-95 (1998), and Zhao et al., Nat. Biotech., 16, 258-61 (1998)). Suitable primers for PCR-based site-directed mutagenesis or related techniques can be prepared by the methods described in, e.g., Crea et al., Proc. Natl. Acad. Sci. USA, 75, 5765 (1978). The application of site-directed mutagenesis to produce novel VEGF variants is described by, e.g., Shortle et al., Ann. Rev. Genet., 15, 288-94 (1981), Keyt et al., J. Biol. Chem., 271, 5638-46 (1996), and Ki et al., J. Biol. Chem., 275(38), 29823-28 (2000).

Other polynucleotide mutagenesis methods useful for producing novel VEGF variant-encoding polynucleotides include PCR mutagenesis techniques (as described in, e.g., Kirsch et al., Nucl. Acids Res., 26(7), 1848-50 (1998), Seraphin et al., Nucl. Acids Res., 24(16), 3276-7 (1996), Caldwell et al., PCR Methods Appl., 2(1), 28-33 (1992), Rice et al., Proc. Natl. Acad. Sci. USA. 89(12), 5467-71 (1992) and U.S. Pat. No. 5,512,463), cassette mutagenesis techniques based on the methods described in Wells et al., Gene, 34, 315 (1985), phagemid display techniques (as described in, e.g., Soumillion et al., Appl. Biochem. Biotechnol., 47, 175-89 (1994), O'Neil et al., Curr. Opin. Struct. Biol., 5(4), 443-49 (1995), Dunn, Curr. Opin. Biotechnol., 7(5), 547-53 (1996), and Koivunen et al., J. Nucl. Med., 40(5), 883-88 (1999)), and recrusive ensemble mutagenesis (REM) (as described in, e.g., Arkin and Yourvan, Proc. Natl. Acad. Sci. USA, 89, 7811-15 (1992), and Delgrave et al., Protein Eng., 6(3), 327-331 (1993)). Alternatively, VEGF variant-encoding polynucleotides can be pre-designed and synthetically produced using techniques such as those described in, e.g., Itakura et al., Annu. Rev. Biochem., 53, 323 (1984), Itakura et al., Science, 198, 1056 (1984), and Ike et al., Nucl. Acid Res., 11, 477 (1983). For example, sequence analysis of a number of VEGF polypeptides (e.g., a group of non-heparin-binding VEGF peptides and/or peptide portions) can be subjected to sequence analysis (e.g., using CLUSTAL-W) to identify an amino acid consensus sequence that can be used to design novel DNAs based on the genetic code (e.g., by subjecting the consensus sequence to reverse translation analysis). Further details regarding the above-described techniques are described in Sambrook et al., and Ausubel et al., supra.

Alternatively, VEGF variants can be generated through directed evolution techniques (e.g., polynucleotide shuffling). Examples of such techniques are described in, e.g., Stemmer, Nature, 370, 389-91 (1994), Cherry et al., Nat. Biotechnol. 17, 379-84 (1 999), and Schmidt-Dannert et al., Nat Biotechnol., 18(7), 750-53 (2000). Preferably, VEGF variant-encoding polynucleotide shuffling is performed in combination with staggered extension (StEP), random primer shuffling, backcrossing of improved variants, or any combination thereof, e.g., as described in Zhao et al., supra, Cherry et al., supra, Arnold et al., Biophys. J., 73, 1147-59 (1997), Zhao and Arnold, Nucl. Acids Res., 25(6), 1307-08 (1997), and Shao et al., Nucl. Acids Res., 26, 681-83 (1998). Alternatively, the incremental truncation for the creation of hybrid enzymes (ITCHY) method (see, e.g., Ostermeier et al., Nat. Biotechnol., 17(12), 1205-09 (1999)) can be applied to combinations of VEGF encoding genes or gene fragments (e.g., to two polynucleotide encoding different non-heparin biding VEGFs (e.g., a human VEGF₁₂₁ and zebrafish VEGF₁₂₁), to two polynucleotides encoding substantially similar (or identical) VEGFs, or to combinations of a non-heparin-binding VEGF and other related protein (e.g., a heparin-binding VEGF)) to produce novel VEGF variant-encoding polynucleotides. Another set of techniques for introducing diversity into a library of homologs are provided in U.S. Pat. No. 6,159,687.

The biological activity of the products of molecular evolution are expected to vary, and, accordingly, some screening for biological activity of the directed evolution product can be required to ensure the peptide portion encoded by the VEGF variant-encoding polynucleotide is suitable for incorporation in the fusion protein and/or fusion protein-encoding polynucleotides of the present invention. Any suitable assay for measuring the desired biological activity of a molecule can be used. The type of assay selected for measuring the biological activity of the VEGF variant will depend on the desired property to be associated with the VEGF variant (e.g., promotion of angiogenesis, bone growth, or wound healing).

Examples of techniques for measuring angiogenesis, and thus for determining the angiogenic potential of angiogenic proteins (e.g., an angiogenic VEGF peptide portion or angiogenic fusion protein), include administering the angiogenic protein or DNA encoding the angiogenic protein (preferably in a suitable vector) in the rabbit or rat hind limb models (using a protocol as described in, e.g., Poliakova et al., J. Thorac. Cardiovasc. Surg., 118(2), 339-47 (1999), Rosengart et al., J. Vasc. Surg., 26(2), 302-12 (1997) et al., J. Cardiovasc. Pharmacol., 27, 91-98 (1996), and/or Takeshita et al., J. Clin. Invest., 93(2), 662-70 (1994), U.S. Pat. No. 6,121,246, or discussed herein in Example 1) and/or the mouse ear model (using a protocol similar to that described in, e.g., Kjosleth et al., Microsurgery, 15(6), 390-98 (1994), or as discussed herein in Example 1). Similar techniques are discussed in, e.g., Takeshita et al., J. Clin. Invest., 93(20), 662-70 (1994). Other assays for assessing the angiogenic potential of an angiogenic factor include performing exercise tolerance tests (as described in, e.g., Fujita et al., Circulation,77(5), 1022-29 (1988), Kornowski et al., Am. J. Cardiol, 81(7A), 44E-48E (1998), and Rosengart et al., Circulation, 100(5), 468-74 (1999)), magnetic resonance imaging (MRI) testing for local perfusion, rest and stress (adenosine) ^{99m} Tc-sestamibi SPECT tests, rest and stress (dobutamine) echocardiograms, gradient echo tests, intravascular ultrasound (IVUS) (as described in, e.g., Oshima et al., Vasc. Med., 3(4), 281-90 (1998)), angiography tests, or any combinations thereof, after administration of the putative angiogenic factor to a tissue (preferably a potentially ischemic or ischemic tissue in a mammalian host). Other quantitative angiogenesis activity assays include the corneal pocket assay, the matrigel angiogenesis/endothelial cell assay, endothelial cell chemotaxis assays, umbilical artery outgrowth assay, choriollantoic membrane development assay, and related assays described in, e.g., Dellian et al., Am. J. Path., 149, 59-72 (1996), Folkman, Cell, 79, 315-28 (1994), O'Reilly et al., Cell, 88, 277-84 (1997), and Ribatti et al., J. Vasc. Res., 34, 455-63 (1997). A more recent assay specifically designed for analytically comparing the angiogenic potential of different factors is described in Wang et al., Int. J. Mol. Med, 6(6), 645-53 (2000). The angiogenesis-inducing capability of a factor also can be determined by comparative measurement of the number of blood vessels, blood vessel density, total blood vessel volume, blood flow measurements, blood pressure ratios, or the like, in a particular tissue to which an angiogenic factor has been administered (as described in, e.g., Sands et al., Cancer Lett., 27(l), 15-21 (1985), Pu, et al., Circulation, 88, 208-15 (1993), Bauters et al., Am. J. Physiol., 267, H1263-71 (1994), Takeshita et al., supra, Bauters et al., Circulation, 91, 2802- 09 (1995), Bauters et al., J. Vasc. Surg, 21, 314-25 (1995), and Witzenbichler et al., Am. J. Pathol., 153(2), 381-94 (1998)). Other useful techniques for measuring angiogenesis include those described in U.S. Pat. Nos. 5,976,782, 5,972,639, and 5,919,759, as well as the in vitro angiogenesis assays described in Tolsma et al., J. Cell Biol., 122, 497 (1993), and Vogel et al., J. Cell. Biochem., 53, 74 (1993).

Bone growth (and thus promotion thereof) can be assessed by assays such as those described in, e.g., Hosh-Choudhery et al., Endocrinology, 137, 331-39 (1996), Urist et al., Proc. Soc. Exp. Biol. Med, 176, 472-75 (1984), Deftos et al., Clin. Chem., 38, 2318-21 (1992), Hassager et al., Metabolism, 40, 205-08 (1991), Kanzaki et al., J. Clin. Endocrinol. Metab., 75, 1104-1109 (1992), and U.S. Pat. Nos. 4,857,456, 5,656,598, 6,071,708, 6,080,779, and 6,150,328. Assays for wound healing activity include those described in Winter, Epidermal Wound Healing, 71-112 (Maibach, HI and Rovee, D T, eds.), as modified by Eaglstein et al., J. Invest. Dermatol., 71, 382-84 (1978).

Other biological activities also or alternatively can be considered in assessing the therapeutic potential of a peptide portion or fusion protein. For example, tissue generation/repair activity, which can be associated with VEGF homologs and fusion proteins, can be assayed using the techniques described in International Patent Applications WO 95/16035, WO 95/05846, and WO 91/07491. Chemotactic activity, which also can be associated with VEGF peptide portions and fusion proteins can be assayed by testing their ability to induce the migration of cells across a membrane or to induce the adhesion of one cell population to another cell population. Suitable assays for movement and adhesion include those described in, e.g., Current Protocols in Immunology, Chapter 6.12 (Colligan et al. eds.), Taub et al., J. Clin. Invest., 95, 1370-76 (1995), Lind et al. APMIS, 103, 140-46 (1995), Muller et al., Eur. J. Immunol., 25, 1744-48 (1994), Gruber et al., J. Immunol., 152, 5860-67 (1994), and Johnston et al., J. Immunol., 153, 1762-68 (1994). Receptor-ligand binding can be determined by the assays described in, e.g., Takai et al., Proc. Natl. Acad. Sci. USA, 84, 6864-68 (1987), Bierer et al., J. Exp. Med., 168, 1145-56 (1988), Rosenstein et al., J. Exp. Med., 169, 149-60 (1989), Stoltenborg et al., J. Immunol. Methods, 175, 59-68 (1994), Stitt et al., Cell, 80, 661-70 (1995), and Chapter 7.28 of Current Protocols in Immunology (Coligan and Kruisbeek eds). Additional assays related to the aforementioned biological activities are described in U.S. Pat. No. 6,099,823.

The VEGF peptide portion can be any suitable size which enables the VEGF portion to exhibit an angiogenic, bone growth promoting, or wound healing promoting activity, or combinations thereof, as desired. Preferably, the biological activity of the VEGF portion is substantially similar to that of a naturally occurring non-heparin-binding VEGF, preferably VEGF₁₂₁ (e.g., about 70% or more, preferably about 80% or more, more preferably about 90% or more, and advantageously at least as much as, and optimally more than, the angiogenesis inducing capacity as VEGF₁₂₁) in a mammalian host. Such biological activity can be measured by any of the methods described herein or their equivalents in the art. Typically and preferably, the VEGF peptide portion will include a VEGF amino acid sequence of less than about 160 amino acid residues, or, more preferably, less than about 150 amino acid residues (e.g., less than about 130 amino acid residues, less than about 120 amino acid residues, less than about 100 amino acid residues, or less than about 90 amino acid residues). Advantageously, the VEGF peptide portion will include a VEGF amino acid sequence of at least 115 amino acid residues, preferably at least about 120 amino acid residues. By non-heparin-binding it is meant that less than about 5% of the VEGF peptide portion of the fusion protein should be bound to heparin-containing sites at a given moment after administration to or expression in a mammalian host (compared to, e.g., about 50-70% binding for VEGF₁₆₅, and about 90-100% for VEGF₁₈₉). More preferably, the VEGF peptide portion exhibits no apparent affinity for heparin, as exhibited by VEGF-C, non-heparin-binding PlGFs, VEGF-E, and, more preferably, VEGF₁₂₁.

Preferably, the VEGF peptide portion exhibits higher affinity for the kinase-insert domain-containing (KDR) receptor (also known as VEGFR-2) than the fms-like tyrosine kinase type 1 (flt-1) receptor (also known as VEGF-R1) or, e.g., the murine flk-1 homolog receptor thereof and/or VEGFR-3 (or Flk receptor). Such VEGF peptide portions are likely to be associated with higher levels of endothelial cell proliferation due to interaction with the KDR receptor without the growth suppressive effects brought about by too much competing flt-1 interaction (see, e.g., Ahmed et al., Lab. Invest., 77(6), 779-91 (1997), for discussion). Endothelial cell proliferation can be measured by any suitable technique, such as the technique described in Olofsson et al., Proc. Natl. Acad. Sci. USA, 93, 2576-81 (1991). Desirably, the angiogenic fusion proteins of the invention generally exhibit higher levels of endothelial cell proliferation upon in vivo expression or administration. Preferably, the VEGF peptide portion exhibits at least about 4×, preferably at least about 5×, and more preferably at least about 6× the affinity for the KDR receptor than the flt-1 receptor. More particularly, it is preferred that the VEGF peptide portion exhibits an apparent affinity for the flt-1 receptor marked by a dissociation constant (Kd) at least about 150 pM, more preferably at least about 175 pM, and even more preferably at least about 200 pM, and optimally not binding to flt-1, while exhibiting an affinity for the KDR receptor marked by a dissociation constant of about 20-30 pM, more preferably about 25-35 pM, and even more preferably about 30 pM. Desirably, the VEGF peptide portion exhibits even less affinity for the flk receptor than the flt receptor, and optimally does not bind the flk receptor at all. VEGF receptor binding can be determined using any suitable technique, such as the VEGF receptor binding assays described in International Patent Application WO 98/49300.

The VEGF peptide portion also desirably exhibits low affinity for neurophilin- 1, neurophilin-2, or both. Preferably, the VEGF peptide portion exhibits an affinity for either or both neurophilins or related proteins (analogs or variants), e.g., marked by a dissociation constant of at least 1,000 pM, more preferably at least 10,000 pM. Ideally, the VEGF peptide portion exhibits an affinity for neurolipin- 1 and neurolipin-2 equal to, or less than, the affinity exhibited by VEGF₁₂₁ (i.e., no apparent affinity). By exhibiting low affinity, or, more preferably, by not binding neurophilins whatsoever, the VEGF peptide portion can avoid undesired interactions which reduce the amount of binding to therapeutic receptors of interest (e.g., the KDR receptor) and avoids interaction with neurophilin-associated tumor cells.

The VEGF peptide portion preferably exhibits a lower level of association with cells and matrix than that of VEGF₁₈₉ and VEGF₂₀₆ (e.g., VEGF₂₀₆ residues 115-139 - see, e.g., Ferrara et al., Endocr. Rev., 13, 18-32 (1992)). In this respect, the VEGF peptide portion preferably lacks a functional “matrix targeting” sequence, a functionally homologous sequence or domain, or any similar sequence. Similarly, the VEGF peptide portion preferably lacks cell association signals, such as the 24 mer motif, Lys Lys Ser Val Arg Gly Lys Gly Lys Gly Gln Lys Arg Lys Arg Lys Lys Ser Arg Tyr Lys Ser Trp Ser Val (SEQ ID NO: 6), common to VEGF₁₈₉ and VEGF₂₀₆.

The VEGF peptide portion preferably comprises a weakly acidic to neural peptide. Thus, the VEGF peptide portion preferably comprises more acidic residues than basic residues. The VEGF peptide portion preferably exhibits less affinity for S-Sepharose than for Q-Sepharose. Desirably, the VEGF peptide portion is associated with a chaperone-associated sequence, which induces interaction with a chaperone with capacity to restore the VEGF peptide portion if damaged by oxidants. Preferably, such chaperon association does not require heparin; for example heparin-dependent Glypican-1 interaction is not desired.

The VEGF peptide portion preferably comprises the C-terminal domain of VEGF₁₂₁ or a closely related domain, particularly the C-terminal cysteine or a counterpart thereof. Thus, the VEGF peptide portion preferably comprises an amino acid sequence falling within the pattern XₕX_{b}XₕXₐX_{b}C, such as Ala Arg Gln Glu Lys Cys (SEQ ID NO: 7), which is a preferred sequence in this pattern, and more preferably comprises a sequence falling within the pattern XₕX_{b}XₕXₐX_{b}CXₐX_{b}XₙX_{b}X_{b}, such as Ala Arg Gln Glu Lys Cys Asp Lys Pro Arg Arg (SEQ ID NO: 8), which is the preferred sequence within this pattern (wherein Xₕ represents a hydrophilic uncharged residue, X_{b} represents a basic residue, Xₐ represents an acidic residue, C represents a cysteine, and Xₙ represents a non-polar uncharged residue) positioned near the peptide portion's C-terminus (e.g., about 30, preferably about 20, and more preferably about 15 amino acid residues or less from the C-terminus). The VEGF peptide portion desirably may comprise, or more typically lack, part or all of a sequence corresponding to the VEGF 6b exon, the VEGF6a exon, or both. Thus, for example, the VEGF peptide can be free of the N-terminal half of the VEGF 6b exon-encoded sequence (SEQ ID NO: 9), the C-terminal half of the VEGF 6b exon-encoded sequence (SEQ ID NO: 10), the core exon 6b-encoded sequence (SEQ ID NO: 11), the core exon 6a-encoded sequence (SEQ ID NO: 12), fragment of the exon 6a-encoded sequence, or sequences which exhibit high levels of identity thereto (e.g., about 80% identity or higher). In some circumstances, a VEGF peptide portion which exhibits a higher level of homology, more preferably identity, to other VEGFs than to a VEGF-B or VEGF-C, particularly to a VEGF-B is preferred.

The VEGF peptide portion is covalently associated with at least one additional non-VEGF peptide portion (also referred to as the “second” peptide portion). The non-VEGF peptide portion can be any suitable peptide portion including a non-VEGF factor, preferably which is capable of promoting angiogenesis, bone growth, wound healing, or any combination thereof, separate from such properties attributed to the VEGF peptide portion (i.e., by directly promoting such biological activities rather than merely augmenting such properties otherwise associated with the VEGF peptide portion). By “non-VEGF” portion, it is meant that the second peptide portion exhibits less than about 20%, preferably less than 10%, and more preferably less than 5% amino acid sequence identity to the VEGF peptide portion, and preferably exhibits at least one distinct biological function from that associated with the VEGF peptide portion, preferably a function related to angiogenesis, bone growth, and/or wound healing. In some circumstances, second peptide portions that exhibit higher levels of angiogenesis inducing activity or bone growth promoting activity than wound healing promoting activity are preferred. Also, in some circumstances, second peptide portions which exhibit less wound healing activity than the wound healing factors described herein can be desirable. Assessments of the angiogenic, bone growth promoting, and wound healing promoting activity of the second peptide proteins can be determined using any of the tests for determining such activities described herein or equivalent such tests. Most preferably, the second peptide portion promotes angiogenesis in vivo (alone or in combination with promoting bone growth and/or wound healing). Often it is desirable that the second peptide portion lacks a functional collagen-binding domain, or more preferably any collagen-binding domain, particularly where the VEGF peptide portion is about 110 amino acids or less in length (e.g., a VEGF₁₁₀ peptide portion).

The second peptide portion can interact with any suitable receptor on any suitable cell type (e.g., a TIE2 receptor in the case of an Ang-1 or ARF second peptide portion) or no receptor at all (e.g., in the case of a SEAP second peptide portion). Typically, the second peptide portion comprises at least one receptor binding domain. Where the second peptide portion interacts with a receptor, the VEGF peptide portion and second peptide portion can have similar or different cellular receptor profiles. Preferably, the VEGF peptide portion and second peptide portion receptor profiles are different (i.e., the VEGF peptide portion binds to at least one peptide portion not bound by the second peptide portion or visa versa). It can be desirable that the VEGF peptide portion and second peptide portion do not commonly interact with the same receptors, thereby increasing the biological activity of the fusion protein. Receptor binding second peptide portions can include any suitable number of receptor binding domains, each domain interacting with any suitable receptor on any suitable cell type. For example, the fusion protein can comprise a second peptide portion that includes, or consists of, one or more endothelial cell-associated receptor binding domains (e.g., a second peptide domain comprising EphrinB2, biologically active fragment thereof, or homolog thereof), and thereby imparts a high level of endothelial cell specificity to the fusion protein (although the VEGF peptide portion alternatively or additional can interact with other cells and be combined with a second peptide portion specific for such cells, for example, a macrophage specific factor). Other suitable heterologous receptor binding domains useful for incorporation in the first peptide portion, second peptide portion, or both portions are discussed further herein.

An “angiogenic VEGF fusion protein” (or angiogenic fusion protein) is any fusion protein of the invention where the second peptide portion comprises a peptide portion which promotes angiogenesis (an angiogenic peptide portion). Typically and preferably the VEGF peptide portion in such fusion proteins also will be angiogenic. “Angiogenesis,” in the context of the invention, encompasses promoting the formation of new blood vessels (also referred to in the art as neovascularization), e.g., by attracting endothelial cells to promote blood vessel sprouting, promoting blood vessel growth from or within existing blood vessels (such as by increasing the size of existing blood vessels or inducing collateral blood vessel growth from existing blood vessels (also known as vasculogenesis)) promoting blood vessel remodeling, promoting blood vessel maturation, and repairing damaged blood vessels (e.g., repairing leaky blood vessels by reducing plasma leakage). Thus, an angiogenic peptide portion can be any sequence of amino acids that induces the initiation of blood vessel growth at a location not otherwise undergoing angiogenesis, enhances or heightens collateral blood vessel growth to a location already undergoing angiogenesis, or both.

The angiogenic peptide portion can be associated with any suitable activity, or combination of activities, involved in angiogenesis. For example, the angiogenic peptide portion can comprise an endothelial cell mitogen (e.g., an aFGF or HGF), a mediator that influences endothelial cell migration or portion thereof (e.g., Del-1), a factor that induces lumen formation and vessel sprouting (e.g., NL1) (including second generation sprout formation, primary sprout formation, capillary loop formation, or combination thereof) or that is associated with inussusceptive or inter calated growth, an endothelial cell differentiation factor, a factor that participates in primary capillary plexus formation, a factor involved in pruning, fusion, or regression of emerging vessel networks, a mediator that influences vessel maturation or remodeling (e.g., a midkine), a mediator that influences vessel wall dilatation or a portion thereof (e.g. an iNOS), an extracellular matrix degradation factor or portion thereof (e.g., a TNF-α), or a factor involved in angiogenesis-related protease secretion, a factor that decreases vascular permeability (e.g., an angiopoietin or midkine), a factor which promotes connection to existing blood vessels, a factor which induces blood vessel branching and/or formation of new capillary networks (i.e., induces non-sprouting angiogenesis or intussusception), a factor that promotes vascular smooth muscle elasticity (e.g., an elastin or a fibrilin (such factors also are useful as wound healing promoting factors, discussed further herein)), a factor involved in vessel differentiation (e.g., formation of a blood barrier or fenestrae), a factor that promotes vessel fusion, a fragment of such factors, or a factor which exhibits any combination of such activities. An angiogenic factor also can be a factor that otherwise influences the amount or size of blood vessels formed or the quality of such vessels (e.g., conduction through such vessels). Thus, the fusion protein can include a second peptide portion that directs/induces blood vessel growth in a different manner than the VEGF peptide portion, thereby improving the angiogenic potential of the protein compared to a protein including or limited to the VEGF peptide portion, second peptide portion, or, preferably, both peptide portions.

Preferably, the fusion protein includes an angiogenic VEGF peptide portion and angiogenic peptide portion which separately act on at least one distinct aspect of angiogenesis from each other. For example, the VEGF peptide portion can act as a endothelial mitogen while the second peptide portion can promote vessel wall maturation, vessel wall dilatation, extracellular matrix degradation, matrix deposition, or combination thereof. Second peptide portions that exhibit blood vessel remodeling activity, blood vessel maturation activity, that reduce vascular permeability, or any combination thereof, are particularly preferred (e.g., an angiopoietin second peptide portion). Preferably, the angiogenic second peptide portion contains a peptide which, upon in vivo administration, exhibits a vascular pattern different than the “hot spot” pattern associated with VEGF₁₂₁ (such as a MK or HBNF) (as described in, e.g., Chourdhuri et al., Cancer Res., 57, 1814-19 (1997)).

The angiogenic second peptide portion can be obtained from, derived from, based upon, include, or consist of any suitable angiogenic peptide. Examples of angiogenic peptides include fibroblast growth factors (FGFs) (e.g., aFGF (FGF- 1) (also known as heparin binding factor 1), bFGF (FGF-2), HST, int-2, FGF-4, FGF-5, FGF-6, and KGF (as discussed in, e.g., Basilico and Moscatelli, “The FGF Family of Growth Factors and Oncogenes” in Advances in Cancer Research, 59, 115-65 (Woude and Klien eds., Academic Press 1992) and U.S. Pat. No. 5,614,496) and their relatives (e.g., HDGFs, as described in, e.g., Klagsbrun et al., Proc. Natl. Acad. Sci. USA, 83, 2448 (1986)), angiogenins (e.g., angiogenin, angiogenin-2, and mAngiogenin-3, as described in, e.g., Strydom et al., Biochemistry, 24, 5486 (1985), Folkman et al., Science, 235, 442 (1987), Bond et al., Biochim. Biophys. Acta, 1162, 177 (1993), Hu et al., Biochem. Biophys. Res. Commun., 197, 682. (1993), Hu et al., Proc. Natl. Acad. Sci. USA, 91, 12096 (1994), and Moenner et al., Eur. J. Biochem., 226, 483 (1994)), pleiotrophin (PTN, also known as HBNF, HB-GAM, HBBM, p18, OSF-1, and HARP, among others as described in, e.g., Kretschmer et al., Growth Factors, 5, 99 (1991), Kretschmer et al., Biochem. Biophys. Res. Commun., 192(2), 420-29 (1993), U.S. Pat. No. 5,270,449, European Patent 0 441 763, and European Patent Application 0 474 979), midkine (MK as described in, e.g., Böhlen and Kovesdi, Prog. Growth Factor Res., 3, 143-57 (1991), Inui et al., J. Peptide Sci., 2, 28-39 (1996), Iwasaki et al., EMBO J., 16, 6936-46 (1997), and U.S. Pat. No. 5,210,026), transforming growth factors (TGFs—e.g., TGF-β), placental growth factors, platelet-derived growth factors (e.g., platelet-derived endothelial cell growth factor and PDGF-BB (Regranex)), ECGF (as described in, e.g., U.S. Pat. No. 4,868,113), Del-1, angiopoietins (e.g., angiopoietin-1 (Ang-1), Ang-2, Ang-3, and Ang-4), angiopoietin homologs (e.g., muscle or liver ALGF (as described in, e.g., International Patent Application WO 99/67382), FRDGs, NL1, NL2, NL3, NL4, NL5, NL6, NL8, zapol, FARF and HFARP (as described in, e.g., Lee et al., Mol. Cells, 11 (1), 100-04 (2001), and Kim et al., Biochem. J., 346 (part 3), 603-10 (2000)), Ang-2A, Ang-2B, and Ang-2C (as described in, e.g., Mezquita et al., Biochem. Biophys. Res. Commun., 275(2), 643-51 (2000)), Ang2(443) (as described in, e.g., Kim et al., J. Biol. Chem., 275(24), 18550-56 (2000)), Ang-6 (as described in, e.g., International Patent Application 01/102429), the angiopoietin related factors described in International Patent Applications WO 00/05241, WO 00/52167, WO 00/37642, WO 00/52167, WO 00/59938, WO 98/05779, WO 99/15653, WO 99/32515, WO 99/32639, WO 99/40193, WO 99/45135, WO 99/62956, WO 99/62925, and WO 99/40193, and variants of such angiopoietins or ARFs (as described in, e.g., Kim et al., J. Biol. Chem., 274, 26523-28 (1999), U.S. Pat. Nos. 5,521,073, 5,643,755, 5,877,289, 5,879,672, 5,972,338, 6,030,831, 6,057,435, and 6,074,873, and International Patent Applications WO 96/11269, WO 96/31598, WO 99/15653, WO 99/32515, WO 99/45135, WO 99/67382, and WO 01/05825), erythropoietin, follistatin, granulocyte colony-stimulating factor (G-CSF), GM-CSF, scatter factor/hepatocyte growth factor (HGF) (as described in, e.g., U.S. Pat. Nos. 6,011,009 and 6,133,231), leptin, insulin like growth factors (IGFs, e.g., IGF-I and IGF-II), endothelial growth factors (EGFs) (e.g., endothelial cell-derived growth factor (ECDGF) and PD-ECGF (as described in, e.g., Matsukawa et al., Biochim, Biophys, Acta, 1314(1-2), 71-82 (1996), Moghaddam et al., Biochemistry, 31, 12141-46 (1992), Miyazono et al., Biochemistry, 28, 1704-10 (1989), and Ishikawa, Nature, 338(6216), 557-62 (1989)), HBEGFs (as described in, e.g., U.S. Pat. No. 6,037,329), epidermal growth factors, connective tissue growth factors (CTGFs—as described in, e.g., U.S. Pat. No. 6,149,916 and Moussad et al., Mol. Genet. Metab., 71(1-2), 276-92 (2000), preferably CTGF-2), matrix metalloproteinases (MMPs) (as described in, e.g., Murphy et al., Matrix Biol., 15(8-9), 511-8 (1997), Baramova et al., Cell Biol. Int., 19(3), 239-42 (1995), and Matrisian, Ann. N. Y Acad. Sci, 732, 42-50 (1994)), tissue inhibitors of metalloproteinase (TIMPs, e.g., vasosten or TIMP-4) (as described in, e.g., Vallamo et al., Human Pathol., 30(7), 795-802 (1999) and Dollery et al., Circ. Res., 84(5), 498-504 (1999)), Delta-3 (as described in, e.g., U.S. Pat. No. 6,121,045), COUP-TFIT, eNOS, iNOS, MCP-1, proliferin, E-selectin, VCAM1, COX-2, HIV-tat, ephrins (e.g., EphB1, EphB2, or EphB4) (as described in, e.g., Yancopoulos et al., Cell, 93, 661-64 (1998) and references cited therein), TWEAK (as described in, e.g., Lynch et al., J. Biol. Chem., 273(13), 8455-49 (1999)), CYR 61 (as described in, e.g., Babic et al., Proc. Natl. Acad. Sci. USA, 95, 6355 (1998)), Fibrin fragment E, PR39 (as described in, e.g., Li et al., Nat. Med., 6(1), 49-55 (2000), and modified by Nat. Med., 6(3), 356 (2000)), tissue plasminogen activator (tPA), urokinase-plasminogen activator (uPA), angiogenic C-x-C chemokines (as described in, e.g., Colville-Nash et al., Mol. Med. Today, 13-23 (1997)), other angiogenic factors described in International Patent Application WO 01/05825, and the AHRs (as described in U.S. Pat. No. 6,121,236). Other angiogenic peptides include cytokines such as tumor necrosis factor-alpha (TNF-α), interleukin-3 (IL-3), and interleukin-8 (IL-8), and transcription factors such as HIF-1 (or HIF-1 α and/or HIF-2α), chimeric HIF factors (e.g., the HIF-1α/VP16 factor described in Vincent et al., Circulation, 18, 2255-61 (2000)), homologs thereof (e.g., EPAS as described in, e.g., Maemura et al., J. Biol. Chem., 274(44), 3165-70 (1999)), fragments thereof, or heterodimers thereof (e.g., a HIF-1α/HIF-2α heterodimer). Preferably, the second peptide portion comprises an angiogenic factor that functions in a manner other than as a transcription factor. Non-peptide angiogenic mediators that can be associated with the fusion protein or co-administered therewith include hormones such as oestrogens and proliferin, alcohols such as glycerol, pyridine derivatives (e.g., nicotinamide), and oligosaccharides such as hyaluronan.

The second peptide portion alternatively can include a homolog of any of the aforementioned angiogenic factors, as well as their naturally occurring homologs, orthologs, paralogs, mutants, or variants. A “homolog” in this sense, and as used herein, specifically with respect to bone growth promoting factors, wound healing promoting factors, and other factors contained in or co-administered with the fusion protein can be any factor meeting one of the four qualifications for VEGF homolog peptide portions described herein (i.e., substantial global or local sequence identity, sequence homology, hydrophobicity conservation, or being encoded by a polynucleotide which hybridizes with the complement of a sequence encoding the naturally occurring factor, or a degenerate sequence thereof). Desirably, homologs of factors described herein further exhibit high levels of weight conservation and structural similarity to their wild-type counterparts, as described above with respect to preferred VEGF homolog peptide portions. Advantageously, such homologs will retain sufficient similarity to react with at least one antibody that reacts with their wild-type counterpart and exhibit similar biological properties (e.g., similar receptor interactions and/or in vivo angiogenic, bone growth promoting, or wound healing activity).

The second peptide portion of an angiogenic VEGF fusion protein also or alternatively can include a peptide portion including a peptide that modulates growth, chemotactic behavior, and/or functional activities of smooth muscle cells (SMCs). The second peptide portion can include any suitable second peptide portion which exhibits such smooth muscle cell-related biological activity. Examples of such smooth muscle cell factors include Activin A, Adrenomedullin, ANF, Angiotensin-2, Betacellulin, CLAF, endothelins, Factor X, Factor Xa, HB-EGF, Heart derived inhibitor of vascular cell proliferation, IFN-γ, IL1, Leiomyoma-derived growth factor (LDGF ), SMC-CF, macrophage-derived growth factor (MDGF), monocyte-derived growth factor, Oncostatin M, Prolactin, Protein S, SDGF (smooth muscle cell-derived growth factor), SDMF (smooth muscle cell-derived migration factor), tachykinins, and Thrombospondin. Homologs of such peptides can also be suitable.

As another alternative, the second peptide portion also or alternatively can include a peptide that modulates growth, chemotactic behavior, and/or functional activities of vascular endothelial cells. Examples of such vascular endothelial cell factors include, in addition to the several factors already discussed herein, Angiotropin (as described in, e.g., Höckel et al., J. Cell Physiol., 133, 1-13 (1987)), AtT20-ECGF, B61, CAM-RF, ChDI, CLAF, ECI, EDMF, EGF, EMAP, Neurothelin, EMMPRIN, Endostatin, Endothelial cell-viability maintaining factor, HGF, HUAF, IFN-γ, K-FGF, LIF, MD-ECI, MECIF, Oncostatin M, PF4, Transferrin, and homologs of such peptides.

Additionally or alternatively, the angiogenic second peptide portion can comprise an anticoagulant or hemostatic process modifier. A hemostatic process modifier can be any suitable protein that effects an aspect of hemostatis (either primary hemostasis, second hemostasis, or both), and desirably is modifies, and most preferably reduces coagulation upon administration or expression. Apart from affecting coagulation, the hemostatic modifier can be any peptide which effects fibrin formation, fibrin deposition, platelet formation, platelet activation, the activity of the fibrinolytic system, tissue factor activation, or any combination thereof, as well as any other suitable hemostatic process. The anticoagulant/hemostatic process modifier second peptide portion can comprise any suitable anticoagulant/hemostatic process modifier, homolog thereof, or fragment thereof. Examples of suitable anticoagulants and hemostatic process modifiers include hirudin, protein C, protein S, tissue factor pathway inhibitors, urokinase, anticoagulant nematode peptide C, bdellins, antistatin, hementin, ornatin, and decorsin (or functionally-related disintegrins). Preferred peptide portions in this respect include an Arg-Gly-Asp adhesion site (RGD site) (as described in, e.g., Krezel et al., Science, 264, 1944-1947 (1994), and references cited therein), a Leu-Asp-Val adhesion site (LDV site) (as described in, e.g., Tselepis et al., J. Biol. Chem., 272(34), 21341-48 (1997), Garat et al., Acta. Anat., 154, 34-35 (1995), Wayner et al., J. Cell Biol., 116(2), 489-97 (1992), and Makarem et al., Biochem. Soc. Trans., 19(4), 380(s) (1991)), a binding site comprising an LDV-like domain (as described in, e.g., Clements et al., J. Cell Sci., 107 (art 8), 2127-35 (1994)), and also or alternatively preferably bind at least one integrin, at least one selectin, at least one lectin, at least one cadherin, at least one thrombin, GP IIb-IIIa, Factor Xa, or combination thereof (preferably, at least one integrin). Preferably, the peptide portion comprises a sequence within the pattern Cys Xaa Xaa Xaa Arg Asp Gly Xaa Xaa Xaa Cys (SEQ ID NO: 13), and more preferably comprises a cysteine rich domain containing at least six cysteines forming three intrachain bonds, desirably within the sequence pattern Cys Xaa₆-l₂ Cys Xaa Cys Xaa₃₋₆ Cys Xaa₃-₆ Cys Xaa₈-₁₄ Cys (SEQ ID NO: 14), where Xaa represents any amino acid (preferably not a cysteine) and subscripted numbers reference possible numbers of such amino acid residues possible between cysteine residues. Desirably, such peptide portions comprise the LAP structural motif (as described in, e.g., Krezel et al, supra), which can be verified by comparison with other LAP structural motifs (e.g., using the techniques described herein with respect to VEGF homolog peptide portions).

The first or second peptide portions can comprise one or more heterologous and/or artificial receptor sites, which preferably change the receptor binding profile of the peptide portion, and more preferably localize the fusion protein (or at least the peptide portion) to a specific cell, group of cells, tissue, or tissues. For example, the fusion protein can include a SEAP second peptide portion which comprises an RGD domain or LDV domain of one of the aforementioned hemostatic modifiers (preferably from decorsin or a homolog thereof), or other integrin binding domain, selectin binding domain, or similar binding domain (e.g., a lamanin, fibrinogen, and/or fibronectin binding domain). Co-administration of fusion proteins comprising an angiogenic, bone growth promoting, or wound healing promoting peptide having such a chimeric receptor (preferably an integrin receptor, which desirably comprises an RGD domain, most preferably a decorsin RGD domain or homolog thereof) also is within the scope of the invention, as is the independent administration of such factors, polynucleotides encoding such factors, and vectors comprising such fusion proteins (as described herein with respect to the VEGF fusion proteins of the invention), preferably to promote angiogenesis, wound healing, or bone growth, in vivo.

Fusion proteins comprising angiogenic second peptide portions including an angiopoietin, an Angiopoietin-related factor (ARF), or homolog thereof, are particularly preferred. An ARF is a protein which exhibits at least about 20% amino acid sequence identity (e.g., at least about 30%, at least about 40%, or at least about 45%) to an angiopoietin, preferably to Ang-1 (SEQ ID NO: 15) (as described in, e.g., U.S. Pat. Nos. 5,521,073, 5,643,755, and 5,879,672), which facilitates angiogenesis in a mammalian host (typically and preferably including promoting and/or inducing vascular sprouting, endothelial cell attraction, and induction of vasculature maturation remodeling). In addition to showing such levels of identity to Ang-1 or another angiopoietin, the ARF peptide portion desirably comprises a fibrinogen-like domain which exhibits at least about 30% identity, more preferably at least about 35%, even more preferably at least about 45%, and advantageously at least about 55% (e.g., at least about 60%, or at least about 65%) amino acid sequence identity to the peptide encoded by polynucleotide KIAA0003 (Nomura et al.—GenBank Accession No. NP001137, as further described in DNA Res., 1(1), 27-35 and 47-56 (1994) (supplement) (1994)) (SEQ ID NO: 16), hereinafter alternatively referred to as KIAA0003-associated peptide or (KAP) (SEQ ID NO: 17) Desirably, the fibrinogen-like domain comprises at least four cysteines, more preferably at least six cysteines, which correspond to the six cysteines present in the fibrinogen like domain of Ang-1. Other suitable fibrinogen-like domains are those meeting the standards set for identifying a fibrinogen-like domain provided in International Patent Application WO 99/45135, which also provides techniques for analyzing sequences to determine if such a domain is present in a particular peptide.

More preferably, the second peptide portion comprises KAP, Ang-1, or an angiogenic fragment of either peptide (preferably a fragment which binds the TIE-2 receptor). Fragments of Ang-1, lacking a significant portion of the N-terminus of Ang-1 are also preferred. Desirably, such truncated Ang-1 peptide portions comprise less than about 50%, more preferably less than about 60%, of the Ang-1 amino acid sequence. Preferably, the Ang-1 truncated peptide portion is truncated in the N-terminal portion of the Ang-1 amino acid sequence. Truncated Ang-1 peptide portions lacking all or part of the predicted Ang-1 alpha helix rich coiled coil domain (SEQ ID NO: 18) (e.g., at least 10%, preferably at least about 50%, and more preferably at least about 90% of either the C-terminus or N-terminus of the domain, or both) are also desirable (other predicted coiled coil domains, including possible Ang-1 coiled coil domains are discussed further herein), as are Ang-1 peptide portions lacking the variable N-terminal domain (SEQ ID NO: 19) (similar modifications can be applied to other angiopoietin peptide portions and angiopoietin related factor peptide portions). Fusion proteins including such truncated Ang-1, or, more preferably, KAP peptide portions, may permit better binding to the KDR and TIE-2 receptors. Fusion proteins that exhibit higher affinity for both the KDR and TIE-2 receptors over full length VEGF-Ang-1 homologs are preferred. Moreover, due to the non-heparin binding nature of the preferred VEGF peptide portion, binding with undesired receptors (e.g., neurophilin-1) is reduced, thereby increasing TIE-2/KDR interaction.

Desirably, an angiopoietin homolog peptide portion (but not typically non-angiopoietin ARFs) will react with at least angiopoietin antibody. Examples of such antibodies are provided in U.S. Pat. No. 6,166,185.

Where the ARF peptide portion does not comprise an Ang-1 peptide portion, KAP peptide portion, or homolog thereof, the ARF peptide portion desirably comprises the fibrinogen-like domain of a peptide (i.e., a domain which is recognized as comprising a fibrinogen-like domain (preferably a domain similar to KAP) through structural analysis, sequence analysis, or combination thereof, preferably as determined through CCD analysis available through the NCBI's BLAST program). Desirably, such a domain exhibits at least about 60% homology, preferably at least about 70% homology (and more preferably identity), to KAP. Preferably, the fibrinogen-like domain is a fibrinogen-like domain of an ARF (e.g., NL4 or Zapol) or artificial homolog thereof (e.g., a mutated NL1 fibrinogen-like domain). Such peptide portions can be naturally occurring ARFs (e.g., an NL1 peptide portion), or a chimeric peptide portion comprising the fibrinogen-like domain of an ARF other than KAP. Any suitable ARF fibrinogen like domain can be incorporated. Examples of suitable fibrinogen like domains include the zapol fibrinogen like domain (FLD) (SEQ ID NO: 20), the Ang2 FLD (SEQ ID NO: 21), the NL3 FLD (SEQ ID NO: 22), the NL4 FLD (SEQ ID NO: 23), the NL8 FLD (SEQ ID NO: 24), human FDRG FLD (SEQ ID NO: 25), the muscle ALGF FLD (SEQ ID NO: 26), the FLS139 FLD (SEQ ID NO: 27), the murine FDRG FLD (SEQ ID NO: 28), the Ang3 FLD (SEQ ID NO: 29), and the Ang4 FLD (SEQ ID NO: 30). Preferred non-KAP fibrinogen-like domains include the fibrinogen-like domain of NL1 (SEQ ID NO: 31) and the fibrinogen-like domain of NL5 (SEQ ID NO: 32). The ARF also can comprise the coiled coil domain from the peptide, or a heterologous coiled coil domain, or a truncated coiled coil domain (e.g., the Ang-2(443) or Ang-2 isoform 1 coiled coil domain (as described in, e.g., Kim et al., J. Biol. Chem. (2000), supra and International Patent Application 98/05779). Alternatively, the ARF portion can comprise an ARF coiled coil domain in combination with a fibrinogen like domain of a non-ARF factor (e.g., a modified fibrinogen C sequence), which interacts with Tie-2, and preferably results in Tie-2 binding, more preferably Tie-2 activation, similar to a wild-type angiopoietin (preferably Ang-1) or ARF (e.g., NL1 or NL5). Synthetic coiled coils, or coiled coils identified in non-ARF peptides (where the ARF peptide portion comprises an ARF fibrinogen like domain) can be incorporated into the ARF peptide portion, preferably which promote multimerization formation (e.g., dimer formation), promote Tie-2 receptor binding, or both. Coiled coil domains can be identified using sequence analysis software, such as the COIL, PAIRCOIL, and PEPCOIL programs, and coiled coil analysis features of the GCG program suite, or through using the PredictProtein server (available at http://www.embl-heidelberg.de/predictprotein/submit_def.html). Alternatively or additionally the ARF peptide portion can act as an apoptosis survival factor for vascular endothelial cells. For example, HFARP second peptide portions are expected to exhibit such activity without binding Tie-2. In some aspects, such non-Tie-2 binding ARFs can be preferred (e.g., where higher levels of VEGF receptor interaction are desired).

Ang-1 peptide portions lacking the multimerization domain function associated with Ang-1 are preferred in certain aspects. For example, a fusion protein in which the multimerization domain of Ang-1 is deleted (or rendered dysfunctional, e.g., through point mutation), but the VEGF peptide portion includes the domain associated with VEGF dimerization, is expected to exhibit greater extracellular mobility in a mammalian host than naturally occurring Ang-1 multimers. Such fusion proteins are further expected to exhibit better in vivo half-life than that of wild-type Ang-1 (e.g., at least twice as long, preferably at least three times as long, and more preferably at least five times as long as a native Ang-1).

The ARF peptide portion can include fragments selected from multiple ARFs (i.e., the ARF peptide portion comprises a fusion protein including two or more ARF peptide portions). Such ARF peptide portions can include any suitable combination of ARF peptide fragments. A preferred chimeric ARF peptide portion in this respect comprises a peptide portion comprising the fibrinogen-like domain of a first ARF fused to the coiled coil domain of a second ARF or other coiled-coil domain containing peptide, which is further fused to the VEGF peptide portion. Illustrations of such peptides, wherein the fibrinogen-like domain peptide portion is provided by KAP, are provided in Examples 6, 9, and 10.

Another preferred group of angiogenic fusion proteins includes a second peptide portion that includes a member of the HBNF-MK family of proteins, homolog thereof, or a fragment thereof, which promotes angiogenesis in a mammalian host. The HBNF-MK family of proteins includes any naturally occurring protein that exhibits at least about 30%, preferably at least about 40%, and more preferably at least about 50% (e.g., at least about 65%, at least about 75%, or even at least about 90% identity) amino acid sequence identity to human HBNF (SEQ ID NO: 33) or MK (SEQ ID NO: 34), preferably to both HBNF and MK, and which are angiogenic, bone growth promoting, or wound growth promoting, when administered to or expressed in a mammalian host. Synthetic homologs of HBNF-MK exhibiting such levels of identity also can be suitable.

The HBNF-MK second peptide portion can include any suitable HBNF-MK peptide or peptide fragment. Preferably, the HBNF-MK peptide portion includes a naturally occurring HBNF, MK, HBNF-MK homolog, or HBNF-MK variant (e.g., a splice variant). Human HBNF, human MK, and more preferably an N-terminal truncated form of human HBNF or MK, which preferably includes about 70% or less, more preferably about 65% or less, and even more preferably about 60% or less (e.g., about 45% or less) of the wild-type HBNF or MK amino acid sequence, are particularly preferred. Typically, deletions in the HBNF or MK sequence required to produce the truncated peptide portion will occur in the N-terminal portion of the full length HBNF or MK protein. Desirably, the HBNF peptide portion will include an amino acid sequence corresponding to (i.e., identical to or highly homologous with) at least about residues 67-109 (SEQ ID NO: 35), more preferably residues 65-118 (encoded by exon 3 of the wild-type HBNF gene) (SEQ ID NO: 36), and even more preferably 65-136 of naturally occurring (mature) HBNF (SEQ ID NO: 37). Advantageously, the HBNF peptide portion comprises a sequence which exhibits at least about 70% homology, more preferably at least about 90% homology, and optimally identity, to the sequence Cys Gly Glu Trp Thr Trp Gly Pro Cys Ile Pro Asn Ser Lys Asp Cys Gly Leu Gly Thr Arg Glu Gly Thr Cys Lys Gln Glu Thr Arg Lys Leu Lys Cys Lys Ile Pro Cys Asn Trp Lys Lys Gln Phe Gly Ala Asp Cys Lys Tyr Lys Phe Glu Ser Trp Gly Glu Cys Asp Ala Asn Thr Gly Leu Lys Thr Arg Ser Gly Thr Leu Lys Lys Ala Leu Tyr Asn Ala Asp Cys (SEQ ID NO: 38). Where the HBNF peptide portion is combined with a heparin-binding VEGF, the HBNF peptide portion may desirably lack the lysine-rich terminal domains of wild-type HBNF (residues 1-21 (SEQ ID NO: 39) and 121-136 (SEQ ID NO: 40) of wild-type HBNF, respectively) or their functional equivalents. Alternatively, in non-heparin-binding VEGF fusion proteins, the inclusion or one or both of these sequences to promote heparin-binding can be desirable.

Desirably, an MK peptide portion will comprise a sequence which exhibits at least about 65% sequence homology, more preferably at least about 75% sequence homology, and ideally identity to SEQ ID NO: 10. Advantageously, an MK peptide portion retains the four C-terminal cysteines which form two intrachain disulfide bridges identical or similar to those present in wild-type mammalian MKs, or a similar set of cysteine residues forming a similar set of intrachain cysteine-cysteine bridges. Thus, the MK peptide portion preferably contains a sequence comprising about residues 60-121 of mature human MK (SEQ ID NO: 41), more preferably about residues 62-104 of human MK (SEQ ID NO: 42), which contain the wild-type MK heparin-binding and dimerization domain, or a sequence exhibiting at least about 65%, preferably at least about 75%, and more preferably at least about 90% homology thereto. For MK peptide portions which lack sequences corresponding to the N-terminal portion of wild-type MK, it is preferred that the MK peptide portion exhibits similar biological activity, e.g., heparin-binding, plasminogen-activator enhancing activity, and neurite extension activity (as described in, e.g., Inui et al., J. Peptide Sci., 2, 28-39 (1996)) as a wild-type MK. Advantageously, the MK peptide portion will exhibit a secondary structure comprising a structure similar to the secondary structure of wild-type MK residues 62-104, a tertiary structure similar to the tertiary structure of wild-type MK residues 62-104, or both (as described in e.g., Iwasaki et al., EMBO J., 16(23), 6936-46 (1997)). Structural similarity using techniques described above with respect to VEGF homolog peptide portions also can be used to determine structural similarity. It may often be desirable that the MK peptide portion exhibits an affinity for nucleolin similar to wild-type MK, or greater than wild-type MK, which can be determined using the techniques described in, e.g., Take et al., J. Biochem., 116, 1063-68 (1994). The MK peptide portion may desirably lack the MK heparin-binding domain, or have a modified domain which permits dimerization but lower affinity to heparin, where the fusion protein comprises a heparin-binding VEGF peptide portion.

Members of the HBNF-MK family that are non-naturally occurring HBNF-MK homologs, e.g., HBNF-MK peptides encoded by polynucleotides produced by mutagenesis, fusion, or directed evolution using naturally occurring HBNF-MK genes, also are contemplated (e.g., HBNF peptide portions lacking the HBNF signal sequence (SEQ ID NO: 43) (such as fusion proteins comprising the VEGF signal sequence or heterologous sequence), MK peptide portions lacking the MK signal sequence (SEQ ID NO: 44), or MK/HBNF peptide portions comprising a HBNF or MK peptide sequence fused to a heterologous signal sequence). Fusion proteins comprising the HBNF, MK, aFGF or other heterologous secreted peptide signal sequence fused to the VEGF peptide portion also are within the scope of the invention.

The HBNF-MK peptide portion typically will bind heparin, particularly in the case of HBNF peptide portions. Thus, while the VEGF portion is typically and preferably non-heparin binding, the second peptide portion can be a heparin binding peptide, although non-heparin binding second peptide portions are typically more preferred.

The HBNF-MK peptide portion, particularly for HBNF or MK homologs or fragment based HBNF-MK peptide portions, preferably retains at least the four cysteine resides forming the two disulfide bonds present in naturally occurring HBNF and MK C-terminal portion (i.e., Cys₆₇-Cys₉₉ and Cys₇₇-Cys₁₀₉, as described in, e.g., Kretschmer et al., supra, Fabri et al., Biochem. Int., 28(1), 1-9 (1992), and Inui et al., J. Peptide Res., 55, 384-97 (2000)), or four cysteine residues corresponding thereto capable of forming structurally similar disulfide bonds. The HBNF-MK peptide portion preferably lacks the domain containing the six N-terminal cysteines present in wild-type HBNF and MK, or their counterparts. Desirably, the HBNF-MK peptide portion will be capable of binding N-syndecan (syndecan-3), syndecan-1, nucleolin, or combination thereof, and most preferably capable of binding syndecan-1, syndecan-3, or both. HBNF-MK homolog peptide portions preferably retain at least about 60%, more preferably at least about 80%, and even more preferably at least about 90% of the 55% of the about 65 naturally occurring HBNF amino acids that are conserved in naturally occurring MK (as described in, e.g., Kretschmer et al., supra). Typically, the HBNF-MK peptide portion will be stable in low pH conditions, in the presence of organic solvents, or both. Also normally, the HBNF-MK peptide portion will exhibit a basic pH. Preferably, the HBNF or MK peptide portion will react with anti-HBNF antibodies, anti-MK antibodies, or both (as described in, e.g., Yeh et al., J. Neurosci., 18(10), 3699-07 (1998), and Obama et al., Anticancer Res., 18, 145-52 (1998)). Also desirably, the HBNF or MK peptide portion will exhibit neurite extension activity, plasminogen activator (PA) activity, or both, as wild-type HBNF or MK (as described in, e.g., Inui et al., supra).

Another preferred angiogenic VEGF fusion protein includes a fibroblast growth factor portion, which preferably is an acidic fibroblast growth factor (aFGF) second peptide portion, which desirably comprises the amino acid sequence of mature human aFGF protein (SEQ ID NO: 45), or homolog thereof, which may or may not be associated with the aFGF propeptide sequence (SEQ ID NO: 46). The aFGF peptide portion can include a naturally occurring aFGF (as described in, e.g., Gautschi-Sova et al., Biochem. Biophys. Res. Commun., 140(3), 874-80 (1986), and Jaye et al., Science, 233(4763), 541-545 (1986)), aFGF fragment, or homolog thereof (e.g., a homolog which meets the conditions described herein for VEGF homologs), such as the aFGF muteins and homologs described in U.S. Pat. No. 5,395,756 and International Patent Application WO 92/11360, preferably which promotes angiogenesis and/or bone growth (most preferably angiogenesis) in a mammalian host. The aFGF peptide portion also can comprise a truncated portion of a wild-type aFGF (e.g., an aFGF comprising at least about 60%, more preferably at least about 75%, of the wild-type aFGF amino acid sequence, such as an aFGF which lacks the wild-type N-terminal acetylation domain (e.g., human aFGF Ala₂) or its counterpart). Desirably, the aFGF peptide portion comprises two cysteines which correspond to the cysteines conserved in human, bovine, rat, hamster, and chicken aFGFs (e.g., human aFGF Cys₃₀ and Cys₉₇) (as described in, e.g., Burgess et al., Mol. Reprod. Develop., 39, 59-61 (1994)). Advantageously, the aFGF portion retains a sequence corresponding to the coding sequence of exon 2 of the human aFGF gene, or a sequence that is at least about 80%, preferably at least about 90%, homologous therewith. Where the FGF peptide portion comprises a cysteine corresponding to aFGF Cys₁₃₁, the surrounding sequence desirably comprises a sequence corresponding to the aFGF residues 127-135 (SEQ ID NO: 47) or a sequence which exhibits high levels of homology to this sequence (e.g., at least 80% homology, and more preferably at least about 90% homology), which comprises and flanks Cys₁₃₁ or its counterpart. aFGF homolog peptide portions desirably exhibit at least about 60% identity to human aFGF. Advantageously, aFGF homolog peptide portions will comprise a sequence falling within the pattern Arg Leu Tyr Cys Xaa₅₋₇ Leu Xaa Xaa Xaa Pro Asp Gly Arg (SEQ ID NO: 48), wherein Xaa represents any amino acid residue and subscripted numbers represent numbers of amino acid residues at a given position, preferably wherein the cysteine residue of the sequence corresponds structurally (e.g., is associated with forming a similar two dimensional or three dimensional protein structure) and/or functionally to Cys₃₀ of human aFGF. Desirably, the aFGF peptide portion comprises a sequence corresponding to residues associated with FGF receptor binding (e.g., the human aFGF ASN₁₂₉ and residues functionally associated therewith). Advantageously, the aFGF peptide portion retains the heparin-binding domain of aFGF, and closely associated residues (e.g., residues 113-116 (Ile₁₁₃ Ser₁₁₄ Lys₁₁₅ Lys₁₁₆ (SEQ ID NO: 49), or residues 24-28 (Lys₂₄ Lys_{25 Pro}₂₆ Lys₂₇ Leu₂₈ (SEQ ID NO: 50), preferably at least residues 113-116, more preferably both sequences (subscripted numbers reference residues positions in human aFGF precursor), or homologs thereof that exhibit similar affinity for heparin).

The angiogenic fusion protein is preferably more angiogenic than a protein including or consisting essentially of either the VEGF peptide portion, the second peptide portion, or, most preferably, more than both a protein including or consisting essentially of either peptide portion. Thus, in vivo administration of the such fusion proteins will typically and preferably result in greater blood flow in the area of administration than the administration of a protein consisting essentially of the second peptide portion in a mammalian host, preferably more than administration of proteins comprising the VEGF portion in a mammalian host, and most preferably more than administration of two proteins respectively corresponding to the two peptide portions. The increased angiogenic potential of the fusion proteins with respect to such non-fusion protein factors can be quantified using any suitable technique described herein or its equivalent in the art.

The in vivo administration of the fusion protein, particularly a fusion protein containing a second peptide portion which reduces plasma leakage (e.g., a MK, Ang-1, or fragment thereof), can result in growth of blood vessels which exhibit less permeability than blood vessels which result from administration of a protein including or limited essentially to the VEGF peptide portion in a mammalian host. For example, fusion proteins where plasma leakage upon in vivo expression or administration result in blood vessels which exhibit about 90% or less, more preferably about 75% or less, and even more preferably about 50% or less (e.g., about 25%) of the vascular permeability exhibited by blood vessels generated by administration of a peptide including or limited essentially to the VEGF peptide portion are contemplated. Blood vessel permeability can be determined using techniques known in the art (see, e.g., Thurston et al., Nat. Med., 6(4), 460-63 (2000), Bates et al., Microcirculation, 6, 83-96 (1999), Thurston et al., Science, 286, 2511-14 (1999), Cox et al., J. Surg. Res., 83(1), 19-26 (1999), Carter et al., Biophys. J., 74(4), 2121-28 (1998), Kendall et al., Exp. Physiol., 80(3), 359-72 (1995), Adamson et al., Microcirculation, 1(4), 251-65 (1994), Yuan et al., Microvasc. Res., 45(3), 269-89 (1993), Olson et al., J. Appl. Physiol., 70(3), 1085-96 (1991), Shibata et al., Jpn. J. Physiol., 41(5), 725-34 (1991), and Kern et al., Am. J. Physiol., 245(2), H229-36 (1983)). Alternatively, the fusion protein can comprise a second peptide portion which does not significantly reduce the VEGF peptide portion-induced permeability. The administration of such fusion proteins, polynucleotides encoding them, and vectors containing such polynucleotides can be advantageous in producing porous (typically peripheral) blood vessels, such as fenestrated capillaries, metaarterioles, blood vessels associated therewith, or blood vessels in association with capillary beds active in filtration, readsorption, or secretion (e.g., the glomerulus), particularly in areas where cardiovascular exchange with tissues is desirable. Administration of such fusion proteins, polynucleotides and vectors also may induce fenestrae opening and exchange. In contrast, in inducing angiogenesis in the brain or other tissues associated with “tight” vessels, administration or expression of a fusion protein which is associated with low levels of vascular permeability is preferred (e.g., angiopoietin second peptide portion fusion proteins, such as a VEGF₁₂₁/angiopoietin fusion protein, or a VEGF₁₈₉/angiopoietin fusion protein, which can be associated with lower incidence of intracerebral bleeding than VEGF₁₂₁ fusion proteins).

Typically and preferably, in vivo expression or administration of the fusion protein is associated with growth of blood vessels which exhibit greater maturity (e.g., blood vessels which exhibit greater density and/or structural similarity to mature mammalian blood vessels) than blood vessels that result from the administration of a protein comprising or limited essentially to the VEGF peptide portion in a mammalian host. More particular examples of maturation events include pericyte coating of forming blood vessels and arterialization of newly formed vessels. Blood vessel maturation can be assessed using any suitable standard. For example, maturity can be observationally assessed by assessing vessel shape, density, luminal regularity, and vessel opening size (as described in, e.g., Bloch et al., FASEB J., 14(5), 2373-76 (2000)). Maturity also can be assessed by signal intensity changes in response to hyperoxia (elevated oxygen) and hypercapnia (elevated carbon dioxide), for example by measuring physiological vasodilatory response to carbon dioxide (as described in, e.g., Gilead and Neeman, Neoplasia, 1 (3), 226-30 (1999)), measuring smooth muscle plasticity, or smooth muscle and non-muscle vascular associated myosin isoform distribution (as described in, e.g., Pauletto et al., Am. J. Hypertens., 7, 661-74 (1994)). Preferably, maturation is determined by assessing recruitment of pericytes to the vasculature, pericyte coating of new vessels, association between the vascular tube and the mural cells, or any combination thereof (as discussed in, e.g., Darland et al., J. Clin. Invest., 103(2), 157-58 (1999), which can be quantified, e.g., by using the microvessel maturation index (MMI) (see, e.g., Goede et al., Lab. Invest., 78(11), 1385-94 (1998)). The in vivo administration of the angiogenic fusion protein to a mammalian host typically will result in a higher number or greater concentration of smooth muscle cells, pericytes, mural cells, total endothelial cells, or any combination thereof, than blood vessels resulting from administration of a protein limited essentially to the first peptide portion. The increase in number of such cells can be detected using techniques known in the art.

In a related sense, blood vessels which result from in vivo administration or expression of the fusion protein desirably exhibit a greater level of blood vessel remodeling than blood vessels which result from administration of a peptide including or limited essentially to the VEGF peptide portion. “Blood vessel remodeling” includes any type of vascular restructuring not associated with maturation, although the events often typically overlap and/or occur simultaneously in living systems. Typical types of blood vessel remodeling events include increase in vascular mass, vessel wall thickening, vessel enlargement or dilation, alteration in capillary density, vascular bed modification, change in vessel tone, or combinations thereof. Blood vessel remodeling can be assessed through stress state and pressure testing, MRI (e.g., as described in Nikol et al., Angiology, 49(4), 251-58 (1998)), in vivo ultrasound imaging or histologic analysis (as described in, e.g., Fung et al., J. Biomech. Eng., 115(4B), 453-59 (1993)), and techniques otherwise used to assess angiogenesis (e.g., gradient echo testing).

In addition to angiogenic fusion proteins, the present invention provides VEGF fusion proteins which alternatively or additionally promote bone growth. “Promoting” includes accelerating the specific biological activity (e.g., bone growth), enhancing the biological activity, or both. In such bone growth promoting fusion proteins, the second peptide portion includes a bone growth promoting factor, bone growth factor homolog, or active fragment thereof. More specifically, the bone growth promoting portion can include any peptide portion that is capable of promoting, or assisting in the promotion of, bone formation, or that increases the rate of primary bone and/or skeletal connective tissue growth or healing, or a combination thereof.

The bone growth promoting portion can include any suitable bone growth promoting factor which can be involved in any aspect of bone growth. Thus, the bone growth promoting portion can include a bone-associated hemorrhaging factor, clot formation factor, granulated tissue ingrowth factor, cartilage formation factor, cartilage turnover factor, callus tissue formation factor, callus tissue remodeling factor (e.g., a cortical and/or trabecular bone development factor), and other osteogenic and/or osteotropic factors. The bone growth promoting portion also can include a mitogen or chemotractant for bone growth associated cells, such as macrophages, fibroblasts, vascular cells, osteoblasts (e.g., HBNFs or TGF-βs), chondroblasts, and osteoclasts. Preferably, the bone growth promoting portion includes a peptide which effects phosphate metabolism, modulates ostocyte activity, otherwise promotes general ossification, osteoblast differentiation, osteopontin expression (e.g., an alkaline phosphatase, preferably a bone specific alkaline phosphatase (BAP) or secreted alkaline phosphatase (SEAP)), or regulating bone mineralization (e.g., an alkaline phosphatase), or the combination thereof, thereby promoting bone healing.

Examples of preferred bone growth factors include the bone morphogenic proteins (BMPs—also sometimes referred to osteogenic proteins (OPs) and COPs, e.g., BMP types 1-12, preferably BMP-2 and homologs/variants thereof, which are variously described in, e.g., U.S. Pat. Nos. 4,795,804, 4,877,864, 4,968,590, 5,011,691, 5,106,748, 5,013,649, 5,108,753, 5,108,922, 5,116,738, 5,166,058, 5,187,076, and 5,141,905, International Patent Applications WO 88/00205, WO 89/09787, and WO 89/09788, and Wozney, Growth Fact. Res., 1, 267-80 (1989)), transforming growth factors (particularly TGFs 1-4, more particularly TGF-α, TGF-β1, and TGF-β2, as described in, e.g., U.S. Pat. Nos. 4,742,003, 4,886,747, and 5,168,051), FGFs (e.g., FGF-1), PDGF, IGF-I, IGF-II, aFGF, bFGF calcitonin, thyroxin, macrophage colony stimulating factor, granulocyte/macrophage colony stimulating factor (GMCSF), epidermal growth factor (EGF), leukemia inhibitory factor (LIF—also known as HILDA and DIA), platelet derived growth factor (PDGF), parathyroid hormone (PTH) insulin-like growth factors (IGF), connective tissue growth factor (CTGF), a hedgehog protein, such as Indian hedgehog (Ihh), parathyroid hormone-related protein (PTHrP), growth and differentiation factor-5 (GDF-5), LIM microvascular protein (LM), latent TGF-binding (LTBP), latent membrane protein-1 (LMP-1), other bone growth promoting factors discussed in International Patent Application WO 01/05825 and alkaline phosphatases (e.g., placental alkaline phosphatase, intestinal alkaline phosphatase, BAP, or SEAP) (as described elsewhere herein). Particularly preferred bone growth promoting factors include the BMPs (e.g., BMP-2), PTH, CTGF, and alkaline phosphatases, particularly BAP (and secreted fragments thereof) and SEAP, and homologs thereof. SEAP peptide portions are especially preferred. The second peptide portion also can include a homolog or fragment of such factors. Preferably, such homolog or fragment peptide portions retain a high level of structural homology to corresponding wild-type factors (e.g., alkaline phosphatase homologs or fragments preferably retain the characteristic phosphatase 10 strand mixed beta sheet structure). In addition to the foregoing factors, the bone growth promoting second peptide portion also can include a bone growth promoting aFGF peptide portion, HBNF-MK peptide portion, or angiopoietin or ARF peptide portion, as described above.

Alkaline phosphatase peptide portions can comprise any suitable alkaline phosphatase. For example, the alkaline phosphatase peptide portion can be a human alkaline phosphatase, a non-human alkaline phosphatase (as described in, e.g., U.S. Pat. No. 5,980,890), or a biologically active fragment or homolog thereof (e.g., a synthetic alkaline phosphatase). In addition to preferably retaining the 10 strand mixed beta sheet structure associated with mammalian alkaline phosphatases, the alkaline phosphatase peptide portion desirably retains a zinc ion binding domain (typically, the carboxyl end of the central beta sheet) and magnesium ion binding domain of a wild-type alkaline phosphatase or homolog thereof, and preferably exhibits zinc and magnesium ion binding within similar binding coordinates (e.g., differing by less than about 0.5 angstroms, preferably less than about 0.1 angstroms) as a wild-type alkaline phosphatase (as described in, e.g., Coleman, Annu. Rev. Biophys. Biomol. Struct., 21, 441-83 (1992)). The alkaline phosphatase peptide portion desirably forms multimers with alkaline phosphatases or other alkaline phosphatase peptide portion-containing fusion proteins, desirably in which at least one multimer member binds a zinc ion in addition to the alkaline phosphatase peptide portion. Also advantageously, the alkaline phosphatase portion exhibits biological activity similar to a wild-type alkaline phosphatase (e.g., substrate binding—as described with respect to select alkaline phosphatases in U.S. Pat. No. 5,783,567 and/or hydrolysis of monophosphate esters, particularly under physiological alkaline conditions (i.e., above a pH of about 7.4)). Preferably, the alkaline phosphatase peptide portion reacts with at least one alkaline phosphatase antibody. Examples of techniques for determining if a BAP will react with a BAP antibody are provided in U.S. Pat. No. 6,201,109, which can be modified with respect to other alkaline phosphatase peptide portions (e.g., human SEAP (SEQ ID NO: 51)) as necessary.

Preferably, the alkaline phosphatase peptide portion exhibits at least about 40% homology (preferably at least about 45% homology, and more preferably at least about 45% identity) to a human placental alkaline phosphatase (e.g., human tissue non-specific alkaline phosphatase), and desirably exhibits at least about 70% weight homology, and more preferably at least about 80% weight homology, to a human wild-type alkaline phosphatase. The alkaline phosphatase may or may not include an alkaline phosphatase signal sequence (such as the human SEAP signal sequence (SEQ ID NO: 52)), and may or may not include the alkaline phosphatase propeptide sequence (e.g., the human SEAP propeptide sequence (SEQ ID NO: 53)). Desirably, the alkaline phosphate portion comprises a sequence exhibiting at least about 60%, more preferably at least about 70%, identity to residues 65-172 of human SEAP (SEQ ID NO: 54). Preferably, the alkaline phosphatase will comprise the sequence Ala Gln Val Pro Asp Ser Ala Xaa Thr Ala Thr Ala Tyr Leu Cys Gly Val Lys Ala Asn (SEQ ID NO: 55) (where X represents any amino acid, preferably an aliphatic uncharged residue, and most preferably a glycine or an alanine), where the serine is phosphorylated under similar conditions as serine 114 of wild-type SEAP, corresponds to the enzymatically active site of the alkaline phosphatase peptide portion, or both. For alkaline phosphatase homolog peptide portions, the peptide portion desirably comprises an amino acid sequence falling within the pattern Thr Asn Val Ala Lys Asn Xaa Ile Met Phe Leu Gly Asp Gly Met Gly Val Ser Thr Val Thr Ala Ala Arg Ile Leu Lys Gly Gln Xaa His His Xaa Xaa Gly Xaa Glu Thr Xaa Leu Xaa Met Asp Xaa Phe Pro Xaa Val Ala Leu Ser Lys Thr Tyr Asn Xaa Xaa Ala Gln Val Pro Asp Ser Ala Xaa Thr Ala Thr Ala Tyr Leu Cys Gly Val Lys Ala Asn Xaa Xaa Thr Xaa Gly Xaa Ser Ala Ala (SEQ ID NO: 56), a sequence falling within the pattern Asn Pro Xaa Gly Phe Phe Leu Xaa Val Glu Gly Gly Arg Ile Asp His Gly His His Glu Gly Lys Ala Xaa Gln Ala Leu Xaa Glu Ala Val Xaa Asp Ala Ile (SEQ ID NO: 57), or a sequence falling within the pattern Glu Asp Thr Leu Thr Xaa Val Thr Ala Asp His Ser His Val Phe Xaa Phe Gly Gly Tyr Thr Xaa Arg Gly Asn Ser Ile Phe Gly Leu Ala Pro Met Xaa Xaa Asp Thr Asp Lys Lys Xaa Xaa Thr Ala Ile Leu Tyr Gly Asn Gly Pro Gly Tyr (SEQ ID NO: 58), and preferably comprises a combination thereof (most desirably all three sequences). Desirably, the alkaline phosphatase peptide portion will be between about 100-700, more preferably between about 200-550, and even more preferably about 500 amino acid residues in length. The alkaline phosphatase may comprise or lack sequences associated with lipid association, glycosylation, or both present in wild-type alkaline phosphatases (e.g., the N₁₄₄-associated glycosylation site and/or D₅₀₆ lipid-binding GPI-anchor site). As the alkaline phosphatase is preferably secreted, it will desirably lack a transmembrane domain (e.g., the SEAP precursor transmembrane domain (SEQ ID NO: 59)), or functional equivalent and typically sequence homolog thereof. Alternatively, the alkaline phosphatase can be rendered in secreted form through small residue changes, including even single residue substitutions, as is known in the art (as discussed in, e.g., Lowe, supra).

Other non-peptide factors which can be associated with the VEGF peptide portion or other portion of the fusion protein involved in bone growth promotion include calucorticoids and estrogen. Such factors can be co-administered with the bone growth promoting fusion protein, as can any one of the aforementioned factors with the fusion protein, polynucleotide, or vector (e.g., co-administration of a BMP and/or a TGF-β can be co-administered with a VEGF/SEAP fusion protein). Co-administration of receptors for such factors (e.g., N-syndecan in association with a bone growth promoting fusion protein containing a HBNF second peptide portion) also is within the scope of the invention.

The invention further provides wound healing fusion proteins. In such aspects, the second peptide portion alternatively or additionally includes a wound healing promoting protein, homolog thereof, or protein/homolog fragment. A wound can include any lesion or injury to any portion of the body of a subject including acute conditions such as thermal burns, chemical burns, radiation burns, burns caused by excess exposure to ultraviolet radiation such as sunburn, damage to bodily tissues such as the perineum as a result of labor and childbirth, injuries sustained during medical procedures such as episiotomies, trauma-induced injuries including cuts and injuries sustained in automobile and other mechanical accidents, injuries caused by bullets, knives, or other weapons, and post-surgical injuries, as well as chronic conditions such as pressure sores, bedsores, conditions related to diabetes and poor circulation, and all types of acne. Commonly encountered wounds in humans include excisional wounds (e.g., tears, cuts, punctures, or lacerations in the epithelial layer, dermal layer, and/or subcutaneous layer of the skin), such as those caused by surgical procedures or from accidental penetration of the skin, lesions due to determatological diseases, burn wounds (such as abrasion bums, surgical burns, and bums from exposure to heat), and dermal skin ulcers (such as decubitus ulcers, diabetic ulcers, venous stasis ulcers, and arterial ulcers). The promotion of wound healing induced by the in vivo presence of the wound healing promoting fusion protein preferably includes the stimulation of new tissue growth, regeneration of connective tissue, or, more preferably, both.

The wound healing promoting portion can include any suitable wound healing promoting factor involved in any aspect of wound healing. For example, the wound healing peptide portion can include a hemostasis (clot formation) factor (e.g., fibrin, fibronectin, or endothelial cell mitogen), wound healing associated inflammation factor (or vascular congestion/tissue edema factor, e.g., an interleukin), contraction factor (e.g., collagen or collagen deposition associated factor), epithelialization factor, connective tissue disposition factor, granulated tissue formation factor, wound remodeling factor (e.g., a collagen cross linking promoting factor or collagen degradation factor), a collagen synthesis stimulating factor (e.g., angiotensin II), a connective tissue proliferation factor, a factor which promotes mitotic activity in the epidermal basal layer, or a factor which exhibits more than one of the aforementioned aspects. Alternatively or additionally, the wound healing promoting portion can include a factor which induces the growth, or is involved in chemotaxis of, cells involved in wound healing such as neutrophils, macrophages, keratinocytes, lymphocytes fibroblasts, SMCs, and other epithelial and/or endothelial cells (e.g., by attracting such cells to the wound bed).

Examples of suitable wound healing promoting factors that can be included in the second peptide portion include extracellular matrix proteins such as collagen, laminin (which also may act as an angiogenic factor), and fibronectin, cell adhesion molecules such as the integrins (e.g., avb3 and avb5), selectin, Ig family members such as N-CAM and L1, and cadherins, cytokine signaling receptors such as the TGF-β type I and type II receptors or the FGF receptor, non-signaling co-receptors such as betaglycan and syndecan, signal transducing kinases, platelet function-associated factors such as von Willebrand factor (vWF), serotonins, platelet activating factor (PAF), and Thromboxane A₂, coagulation factors such as kininogen, kallikrein, thromboplastin (Factor III), prothrombin and thrombin (Factor II), fibrinogen and fibrin (Factor I), and fibrin-stabilizing factor, and cytoskeletal proteins such as talin and vinculin. Additional examples of specific wound healing promoting factors include the bFGFs (e.g., FGF-1 and FGF-2 as described in, e.g., Slavin et al., Cell Biol. Int., 19, 431-444 (1995)), EGFs, PDGFs, PGF, IGF, calrectulin, CTGF, collagen, keratinocyte growth factor (KGF), tissue transglutanimase (TG), clotting factors (e.g., fibrinogen, prothrombin, and thrombin), M-CSF, growth hormones or somatotrophins (e.g., hGH) Factor VIII, Factor IX, EPO, tPA, transforming growth factors (particularly TGF-β), activins, inhibins, PTH, and alkaline phosphatases (e.g., placental alkaline phosphatase, intestinal alkaline phosphatase, bone alkaline phosphatase (BAP (also sometimes referred to as B-ALP, which also may be present in liver and kidney tissues)) and/or non-tissue specific alkaline phosphatase, germ cell alkaline phosphatase, or placenta alkaline phosphatase-derived secreted alkaline phosphatase (SEAP) (as described in, e.g., Coleman, Annu. Rev. Biophys. Biomol. Struct., 21, 441-83 (1992), Lowe, J. Cell Biol., 116 (3), 799-807 (1992), Fishman, Clin. Biochem., 23(2), 99-104 (1990), Kishi et al., Nucleic Acids Res., 17(5), 2129, Harris, Clin. Chem. Acta, 186, 133-150 (1989), Berger et al., Gene, 66, 1-10 (1988), Millan, Anticancer Res., 8, 995-1004 (1988), Weiss et al., J. Biol. Chem., 263(24), 12002-12010 (1988), Coleman et al., Adv. Enzymol., 55, 381 (1983), and U.S. Pat. Nos. 4,659,666 and 5,434,067), and the recombinant and modified alkaline phosphatases, such as those described in U.S. Pat. Nos. 5,081,227, 5,773,226, and 5,821,095), homologs thereof, and fragments thereof (e.g., a secreted alkaline phosphatase derived from a BAP or non-tissue specific alkaline phosphatase). Additionally, wound healing promoting aFGF peptide portions, HBNF-MK peptide portions, and angiopoietin/ARF peptide portions also can form, or be included in, the second peptide portion. Particularly preferred wound healing factors include the PDGFs, aFGF, HBNFs, MKs, TGF-β, and CTGFs, of which HBNFs, MKs, TGF-β, and CTGFs are most preferred. The wound healing promoting portion can, and typically will, lack a functional collagen binding domain (e.g., a collagen binding domain rendered dysfunctional by truncation or mutation), or any collagen binding domain, especially where the non-heparin-binding VEGF peptide portion comprises a peptide portion of 110 amino acids or less (e.g., a VEGF₁₁₀ peptide portion). For example, HBNF, MK, SEAP, and aFGF peptide portions will not typically include such domains. Where the second peptide portion comprises such a domain (e.g., in the case of a von Willebrand factor peptide portion), the VEGF peptide portion is preferably at least about 115 amino acids in length, more preferably between about 115-165 amino acids in length, and even more preferably about 120 amino acids in length (e.g., a VEGF₁₂₀ or VEGF₁₂₁ peptide portion).

Non-peptide factors such as glucocorticoids, adenosine diphosphate, and vitamins A, C, E, and K, can also aid in wound healing. Co-administration of such factors with the wound healing fusion protein (or polynucleotide encoding the wound healing fusion protein) can further facilitate wound healing. Preferably, the wound healing fusion protein will prevent or decrease scar formation, such as keloids and hypertrophic scars, as well as decreasing the extent of scar tissue formation either internally or externally, as applicable.

As indicated above, in some contexts a fusion protein consisting of a heparin-binding VEGF peptide portion is preferred over fusion proteins comprising a non-heparin-binding VEGF peptide portion. Accordingly, such fusion proteins also are provided by the invention. In general, the principles applicable to the non-heparin-binding VEGF peptide portion are also applicable to such heparin-binding VEGF peptide portions, except with respect to factors such as mobility (discussed with respect to non-heparin-binding VEGF fusion proteins below), pH (as discussed above), and/or protein interactions (e.g., neurophilin interactions or VEGF receptor interactions), which typically will vary from those described above with respect to non-heparin-binding VEGF peptide portions (i.e., by exhibiting biological activity similar to heparin-binding VEGFs, such as VEGF₁₈₉ or VEGF₁₆₅). The heparin-binding VEGF peptide portion can comprise any suitable heparin-binding VEGF (e.g., a VEGF₁₈₉ or homolog thereof). VEGF₁₆₅, heparin-binding fragments thereof, and homologs thereof, are preferred wild-type and wild-type-derived heparin binding VEGF peptide portions components. Other advantageous heparin-binding VEGFs include VEGFs derived from VEGF₁₂₁, which typically generated through addition of the heparin-binding domain of another VEGF, such as VEGF₁₈₉ or an artificial heparin-binding domain. Examples of such VEGFs include VEGF_{121.2} (SEQ ID NO: 60) and VEGF_{121.3} (SEQ ID NO: 61), which include a heparin binding domain derived from VEGF₁₈₉, and VEGF_{121.5} (SEQ ID NO: 62) and VEGF_{121.6} (SEQ ID NO: 63), which include artificial heparin binding domains. Such VEGFs may exhibit higher heparin binding than VEGF₁₆₅ and, thus, can be advantageous in aspects where a heparin binding VEGF peptide portion is desirable. Similar modified heparin-binding VEGFs, which also can be suitable for incorporation in such fusion proteins, are described in International Patent Application WO 98/36075.

Preferably, for fusion proteins comprising a heparin-binding VEGF peptide portion, the second peptide portion is not an Angiopoietin-related factor, and more preferably not an angiopoietin. Moreover, the second peptide portion in such aspects desirably does not consist of a heparin-binding peptide which would interfere with the desired VEGF-heparin interaction. Thus, for example, the second peptide portion preferably is not a FGF peptide portion. Such fusion proteins can comprise any of the second peptide portions described herein, and often will consist of a wound healing or bone growth promoting second peptide portion (e.g., versus an angiogenic second peptide portion).

Heparin-binding VEGF fusion proteins can be administered to the host similar to the non-heparin-binding fusion proteins otherwise described herein. For example, such fusion proteins can be administered by preparing a vector, preferably an adenoviral vector such as those described elsewhere herein, comprising a polynucleotide encoding the heparin-binding VEGF fusion protein.

The VEGF peptide portion and second peptide portion can be associated in any suitable manner. Typically and preferably, the first and second peptide portions will be covalently associated (e.g., by means of a peptide or disulfide bond). The first and second peptide portions can be directly fused (e.g., the C-terminus of the VEGF peptide portion can be fused to the N-terminus of the second peptide portion through a peptide bond between the two portions). The fusion protein can include any suitable number of modified bonds, e.g., isosteres, within or between the peptide portions. Alternatively, the fusion protein can include a peptide linker between the peptide portions that includes one or more amino acid sequences not forming part of the biologically active peptide portions. Any suitable peptide linker can be used. The linker can be any suitable size. Typically, the linker will be less than about 30 amino acid residues, preferably less than about 20 amino acid residues, and more preferably about 10 or less amino acid residues. Typically the linker will predominantly consist of neutral amino acid residues. Suitable linkers are generally described in, e.g., U.S. Pat. Nos. 5,990,275 and 6,197,946, and European Patent Application 0 035 384.

The linker can include one or more cleavage sites to promote separation of the peptide portions if desired under specific conditions (e.g., exposure to certain proteolytic enzymes). Examples of such cleavage sites include the Ile Glu Gly Arg linker sequence (SEQ ID NO: 64), which is cleaved by Factor Xₐ protease. Other sites can include sequences which are cleaved by, for example, trypsin, enterokinase, collagenase, and thrombin. Alternatively, the cleavage site in the linker sequence can be a site capable of being cleaved upon exposure to a selected chemical or chemical state, e.g., cyanogen bromide, hydroxylamine, or low pH. Additional examples of suitable cleavable linkers are provided in U.S. Pat. No. 4,719,326. Other suitable types of linkers are described in, e.g., U.S. Pat. No. 6,010,883.

Cleavage, particularly when followed by degradation of one of the peptide portions, can offer a technique for providing a higher level of one of the two peptide portions, when desired. For example, a higher concentration of angiopoietin/ARF or HBNF-MK peptide portion can be desired after the induction of angiogenesis, to promote blood vessel maturation and/or reduce plasma leakage. In this regard, polynucleotides or vectors encoding for such cleavage factors that are expressed under different conditions than a polynucleotide encoding the fusion protein can be administered in association therewith so as to separate the peptide portions under desired conditions. Alternatively, such cleavage factors can be administered to or near an area of fusion protein administration or expression. Such cleavage sequences also can be introduced (or, if already present, exploited) in the VEGF peptide portion or second peptide portion. For example, a polynucleotide can encode a heparin-binding VEGF peptide portion (e.g., VEGF₁₈₉), which can be cleaved in order to render the VEGF peptide portion a non-heparin-binding VEGF peptide portion (e.g., by using the proteolytic cleavage sites naturally occuring VEGF₁₈₉). Typically and preferably, the first and second peptide portions will be directly fused, or separated by a non-cleavable linker of less than about 10 amino acid residues (e.g., 1-5 amino acid residues), so as to retain the improved/synergistic qualities of the fusion protein (e.g., greater mobility and/or larger in vivo half life) as desired herein.

Linkers which reduce the immunogenicity of the fusion protein in its intended recipient are preferred. Any linker which reduces the immune response of the intended recipient of the fusion protein is suitable in this respect. Typically, a flexible linker, which does not interfere with the tertiary structure of the first peptide portion, the second peptide portion, or, most preferably, both peptide portions is used. By not interfering with the tertiary structure of the peptide portion(s), the flexible linker will not configure the fusion protein such that foreign epitopes are presented to the target's immune system. Furthermore, the flexible linker is desirably immunological inert in the host system, and addition of it to the fusion protein desirably does not produce epitopes resulting in a strong immunological host response against the fusion protein, and desirably eliminates any sequences that might result in such immune response from the otherwise direct fusion of the first and second peptide portions. Any flexible linker can be used. Typically and preferably the flexible Gly₄Ser₃ linker or derivative thereof (i.e., a linker comprising the sequence Gly Gly Gly Gly Ser Ser Ser (SEQ ID NO: 65) is used in such fusion proteins. The use of such flexible linkers is described in, e.g., McCafferty et al., Nature, 348, 552-554 (1990), Huston et al., Proc. Natl. Acad. Sci. USA, 85, 5879-5883 (1988), Glockshuber et al., Biochemistry, 29, 1362-1367 (1990), and Cheadle et al., Molecular Immunol., 29, 21-30 (1992). Other glycine-rich flexible linkers also can be suitable, such as the Pro Ggly Ile Ser Gly Gly Gly Gly Gly linker (SEQ ID NO: 66), described in Guan et al., Anal. Biochem., 192(2), 262-67 (1991), the Gly Gly Gly Gly Ser Gly Gly Gly Gly Ser Gly Gly Gly Gly Ser linker (SEQ ID NO: 66), described in Huston et al., Proc. Natl. Acad. Sci. USA, 85, 5879-5883 (1988), and the Glu Gly Lys Ser Ser Gly Ser Gly Ser Glu Lys Glu Phe linker (SEQ ID NO: 67), described in Bird et al., Science, 242, 423-26 (1988). Other suitable flexible linkers include the immunoglobulin hinge linkers (as described in, e.g., U.S. Pat. Nos. 5,672,683 and 6,165,476), and helical peptide linkers (as described in, e.g., U.S. Pat. No. 6,132,992).

Alternatively, where the first and second peptide portions are directly fused, the fusion can be designed such that the intersection of the first and second peptide portions does not generate a sequence which results in a strong immune response against the fusion protein (e.g., as compared to the direct fusion of the wild-type peptide portions). Such determinations can be made by using algorithms which identify MHC class I and MHC class II epitope sequences (preferably through the use of bioinformatics software incorporating such algorithms or through the use of databases which provide listings of such epitopes identified with such algorithms). Any suitable algorithm, database, or program can be used. Examples of such algorithms, programs, and databases include the EPIMER/EPIMAX algorithm developed at the Brown University School of Medicine, the BONSAI algorithm developed at Stanford University, the TEPITOPE algorthim, the Zycos, Inc. “EPIQUEST” database, the SYFPEITHI program (which applies the algorithm of Rammensee et al.), the MAPPP program (available at http://www.mpiib-berlin.mpg.de/MAPPP/addquery.html), and the BIMAS program (available at http://bimas.dcrt.nih.gov/molbio/hla_bind/), which are variously described in, e.g., Altuvia et al., Mol. Immunol., 31, 1-19 (1994), Brusic et al., Nuc Acids Res., 22, 3663-3665 (1994), Hammer et al., J. Exp. Med, 180, 2353-2358 (1994), Parker et al., J. Immunol., 152, 163-175 (1994), Sturniolo et al. Adv. Immunol., 66, 67-100 (1997), and Cunha-Neto, Braz. J. Med. Biol. Res., 32(2), 199-205 (1999). The amino acid sequence which would result upon the production or expression of the fusion protein of interest, particularly the area where the first and second peptide portions are bonded (i.e., the “fusion point”), and surrounding region (typically about 15 or less, more typically about 10 amino acid residues or less, in both directions from the fusion point), can be inputted into such a program, referenced against such databases, or analyzed by similar technique, to determined whether the sequence would result in an undesired host immune response (e.g., formation of a complex with an MHC class I molecule, MHC class II molecule, or both). Thus, the invention provides a VEGF fusion protein wherein the first peptide portion, second peptide portion, or both portions, lack one or more amino acid residues corresponding to residues in their wild-type counterparts near the fusion point of the first and second peptide portions, typically within about 20 amino acids or less, more typically within about 10 amino acids or less of the fusion point. In such fusion proteins, the C-terminus of the first peptide portion, N-terminus of the second peptide portion (or visa versa depending upon the orientation of the first and second peptide portions in the fusion protein), or both termini in both portions, will thus lack one or more amino acid residues occurring in their wild-type counterparts, where the lack of such residues results in a lower level of host immune response against the fusion protein upon expression or administration (e.g., by reducing the immunogenicity of, or eliminating, sequences that result in a host cellular or humoral (typically cellular) immune response against the expressed or administered fusion protein). The residues that would result in the immunologically-undesirable amino acid sequence can be removed either through deletion or through non-immunologically equivalent substitutions (which typically will be non-homologous in nature). Typically and preferably about 15 or less, more typically about 5 or less of the residues at the fusion of the first and second peptide portions will require deletion or substitution. In some fusion proteins, even a single deletion or substitution will result in the desired reduction in the immunogenicity of the sequence formed by the fusion of the first peptide portion and second peptide portion. By “corresponding” in this context, it is meant that the deleted/substituted residue is homologous to, or more typically identical to, a sequence occurring in the wild-type peptide, and would align with the residue in the peptide portion's wild-type counterpart in an optimal alignment. Similar techniques can be applied to fusion proteins that contain a linker if necessary. Immunogenicity testing of the fusion protein or polynucleotides of the invention also can be assessed using any suitable immunogenicity model prior to administration to the target, particularly where the target of administration is a human, to determine whether the area of fusion will exhibit an acceptable level of immunogenicity upon in vivo administration or expression.

Other techniques for reducing immunogenicity of the fusion protein, polynucleotide, or vector (including the vector composition and fusion protein composition) of the invention can be used in association with the administration of the fusion protein, polynucleotide, vector, or related compositions (e.g., the vector compositions of the invention). For example, the techniques provided in U.S. Pat. No. 6,093,699 may reduce such an immune response to the fusion protein.

Where a linker is incorporated into the fusion protein, the presence of the linker preferably does not impede the biological activity of the first peptide portion or second peptide portion, and more preferably of either peptide portion, and more desirably enhances the biological activity of the separate peptide portions over a direct fusion of the peptide portions (e.g., the promotion of angiogenesis, bone growth, wound healing, VEGF receptor binding, Tie-2 receptor binding, multimerization, etc.). Examples of techniques used to assess the effect of linker sequences on the biological activities of fusion proteins are described in, e.g., Newton et al., Biochemistry, 35, 545-553 (1995), which can be modified as appropriate for the fusion proteins of the invention (e.g., using the biological assays described elsewhere herein). It will typically be advantageous for the linker to permit the first peptide portion, second peptide portion, or both portions, to exhibit a secondary and/or tertiary structure similar to that of their native peptide counterparts, which can be assessed using techniques provided herein or which are similar to such techniques.

In addition to the VEGF peptide portion and second peptide portion, the fusion protein can include any suitable number of peptide portions in any suitable arrangement. For example, the fusion protein can include 3, 5, 10, or more peptide portions (e.g., including 2, 3, 4, or more angiogenic peptide portions, or angiogenic portions combined with SMC facilitating peptide portions). In such aspects, the peptide portions can include one or more repeated peptide portions or can be limited to several different peptide portions. Preferably, the fusion protein contains only non-heparin binding VEGF peptide portions. Thus, the fusion protein can be any size suitable to promote angiogenesis, bone growth, wound healing, or combination thereof. In this respect, the term “protein” as used herein is considered to be interchangeable with the terms “peptide” and “polypeptide” to refer to a molecule comprising a plurality of amino acid residues. Typically, the fusion protein will comprise about 200-1000 amino acid residues, more typically about 400-700 amino acid residues, and typically will weigh about 400-2000 kDa, more preferably about 50-100 kDa.

The first peptide portion can include any number of other elements or modifications, e.g., additional amino acid sequences or other peptide fragments, as long as the biological functions (e.g., bone growth promoting ability) of the fusion protein are not substantially diminished (i.e., not diminished by more than about 20%, preferably not more than about 10%, and even more preferably not at all) over a fusion protein lacking such additional elements. Examples of such elements include sequences encoding proteins for post-translational modification or for binding to a small molecule ligand.

Fusion proteins produced in recombinant host cells using the techniques described herein (or their equivalents in the art) are often subject to post-translational modifications (as a consequence of the selected host cell and/or as a desired modification (e.g., one that increases its therapeutic potential). Such post-translationally modified fusion proteins are contemplated. Examples of common post-translational modifications include carboxylation, glycosylation, hydroxylation, lipid or lipid derivative-attachment, methylation, myristylation, phosphorylation, and sulfation. Other post-translational modifications include acetylation, acylation, ADP-ribosylation, amidation, covalent attachment of flavin, covalent attachment of a heme moiety, covalent attachment of a nucleotide or nucleotide derivative, covalent attachment of phosphotidylinositol, cross-linking, cyclization, disulfide bond formation, demethylation, formylation, GPI anchor formation, iodination, oxidation, proteolytic processing, prenylation, racemization, selenoylation, transfer-RNA mediated addition of amino acids to proteins such as arginylation, and ubiquitination. Similar modifications are described in, e.g., Creighton, supra, Seifteretal., Meth. Enzymol., 182, 626-646 (1990), and Rattan et al., Ann. N.Y. Acad. Sci., 663, 48-62 (1992). Moreover, the fusion proteins of the invention include both methionine-containing and methionineless N-terminal variants of the fusion proteins described herein. The nature and extent of post-translational modifications is largely determined by the host cell's posttranslational modification capacity and the modification signals present in the polypeptide amino acid sequence. For instance, glycosylation often does not occur in bacterial hosts such as E. coli. Accordingly, when glycosylation is desired, a polypeptide should be expressed in a glycosylating host, generally a eukaryotic cell (e.g., a mammalian cell or an insect cell). Post-translational modifications can be verified by any suitable technique, including, e.g., x-ray diffraction, NMR imaging, mass spectrometry, and/or chromatography (e.g., reverse phase chromatography, affinity chromatography, or GLC). The fusion protein or portion thereof also or additionally can comprise one or more modified amino acids, non-naturally occurring amino acids (e.g., β amino acids), or amino acid analogs, such as those listed in the Manual of patent Examining Procedure § 2422 (7th Revision—2000), which can be incorporated by protein synthesis, such as through solid phase protein synthesis (described in, e.g., Merrifield, Adv. Enzymol., 32, 221-296 (1969)).

In view of the capacity for post-translational modifications and the desirability of fusion protein extracellular mobility, a common additional element present in the fusion protein is a signal sequence, which directs either organelle trafficking (e.g., an endoplasmic reticulum trafficking signal as described in, e.g., U.S. Pat. No. 5,846,540) and/or cell secretion. Such sequences are typically present in the immature (i.e., not fully processed) form of the fusion protein, and are subsequently removed/degraded to arrive at the mature form of the protein. Both naturally occurring and heterologous signal sequences are suitable (e.g., a secretion sequence associated with the protein incorporated in the second peptide portion as discussed herein). For example, a heterologous signal sequence (e.g., a HBNF signal sequence, alkaline phosphatase signal sequence, fusion thereof, or homolog thereof) can be fused to the N-terminus of the VEGF peptide portion to facilitate the secretion of the fusion protein from recombinant host cells. Alternatively, the VEGF-A secretion signal sequence Met Asn Phe Leu Leu Ser Trp Val His Trp Ser Leu Ala Leu Leu Leu Val Leu His His Ala Lys Trp Ser Gln Ala (SEQ ID NO: 68) (which is retained in all VEGF-As, e.g., VEGF₁₂₁), or a portion thereof, can be used, preferably bound to the N-terminus of the VEGF peptide portion. Such sequences will necessarily vary with the host in which the fusion protein is expressed. Examples of heterologous secretion sequences include STII or Ipp for E. coli, alpha factor for yeast, and viral signals such as herpes gD for mammalian cells. Further examples of signal sequences are described in, e.g., U.S. Pat. Nos. 4,690,898, 5,284,768, 5,580,758, 5,652,139, and 5,932,445. Additional signal sequences can be identified using skill known in the art. For example, sequences identified by screening a library can be analyzed using the SignalP program (see, e.g., Nielsen et al., Protein Engineering, 10, 1-6 (1997)), or similar sequence analysis software capable of identifying signal-sequence-like domains, or by otherwise analyzing the sequences for features associated with signal sequences, as described in, e.g., European Patent Application 0 621337.

In view of the above, it should be clear that the fusion proteins of the invention include both mature (fully processed) and immature (nascent) peptide portions, particularly where such fusion proteins are produced through the expression of a polynucleotide of the invention. In this respect, a peptide portion of the fusion protein can comprise one or more “propeptide” regions, which are removed during processing. Accordingly, nucleotide sequences encoding such propeptide portions along with the “mature” amino acid sequence associated with the peptide portion are within the scope of the invention.

Other sequences that can be included in the fusion protein include binding regions, such as avidin or an epitope, which can be useful for purification and processing of the fusion protein. Examples of such sequences are described in, e.g., International Patent Application WO 00/15823. In addition, detectable markers can be attached to the fusion protein, so that the traffic of the fusion protein through a body or cell can be monitored conveniently. Such markers may include radionuclides, enzymes, fluorophores, small molecule ligands, and the like.

Recently, the production of fusion proteins comprising a prion-determining domain has been used to produce a protein vector capable of non-Mendelian transmission to progeny cells (see, e.g., Li et al., J. Mol. Biol., 301(3), 567-73 (2000)). The inclusion of such prion-determining sequences in the fusion protein is contemplated, ideally to provide a hereditable protein vector comprising the fusion protein that does not require a change in the host's genome.

The mature fusion protein also can include additional peptide portions which act to promote stability, purification, and/or detection of the fusion protein. For example, a reporter peptide portion (e.g., green fluorescent protein (GFP), β-galactosidase, or a detectable domain thereof) can be incorporated in the fusion protein. Purification facilitating peptide portions include those derived or obtained from maltose binding protein (MBP), glutathione-S-transferase (GST), or thioredoxin (TRX). The fusion protein also or alternatively can be tagged with an epitope which can be antibody purified (e.g., the Flag epitope, which is commercially available from Kodak (New Haven, Conn.)), a hexa-histidine peptide, such as the tag provided in a pQE vector available from QIAGEN, Inc. (Chatsworth, Calif.), or an HA tag (as described in, e.g., Wilson et al., Cell, 37, 767 (1984)).

The fusion protein also can include a heterologous (i.e., non-VEGF and non-second peptide portion) multimerizing domain or multimerizing component (i.e., a domain of one of the peptide portions or a separate peptide portion which facilitates multimer formation), which permits the fusion protein to form multimers (including dimers). Examples of heterologous multimerization domains are the human immunoglobulin (IgG) multimerization domains (see, e.g., European Patent Application 0464533) and IgG-derived domains (e.g., the Fc domain as described in, e.g., Johanson et al., J. Biol. Chem., 270, 9459-71 (1995)). Additional modified IgG multimerizing domains and other multimerizing domains are described in International Patent Application WO 00/37642 and the references cited therein.

Typically and preferably, the fusion protein, particularly with respect to angiogenic fusion proteins, will contain a multimerization domain, and thus form multimers (e.g., dimers), which can be either fusion protein homodimers or heterodimers formed with other proteins, preferably with other angiogenic, bone growth promoting, or wound healing promoting factors. It is sometimes desirable that the multimerization domain is obtained from an angiogenic, bone growth promoting, or wound healing promoting peptide (e.g., from the N-terminal portion of an angiopoietin, ARF, or a portion of a TGF-β containing the TGF-β dimerization domain), or a peptide associated with such biological activities (e.g., a vitronectin) versus other multimer-forming peptides, e.g., an IgG.

The fusion protein can include any suitable multimerization domain which results in the formation of any suitable multimer. The fusion protein multimer can be a heteromultimer (e.g., a heterodimer) or a homomultimer (e.g., a homodimer). Homomultimers or heteromultimers which involve association with proteins that exhibit significant levels (e.g., at least about 70%, preferably at least about 80%, and more preferably at least about 90%) sequence identity to the VEGF peptide portion or second peptide portion are preferred (e.g., a heteromultimer formed between the fusion protein and a VEGF₁₂₁ or Ang-1). Other heteromultimers also can be suitable, but testing of novel multimer combinations (e.g., using the techniques described herein or their equivalent) can be necessary to determine whether the multimer effectively induces angiogenesis, bone growth, wound healing, or other desired activity. Such analysis is commonly performed in the art (see, e.g., DiSalvo et al., J. Biol. Chem., 270, 7717-23 (1995), Cao et al., J. Biol. Chem., 271, 3154-62 (1996), and Olofsson et al., Proc. Natl. Acad. Sci. USA, 93, 2567-81 (1996)).

In some aspects, the fusion protein contains a multimerization domain which permits dimer formation without permitting formation of higher order multimers. For example, fusion proteins that include the dimerization domains of VEGF₁₂₁ or TGF-β can limit multimerization to dimmer formation.

The fusion protein can be further modified or derivatized in any suitable manner (e.g., by reaction with organic derivatizing agents). For example, the fusion protein can be linked to one or more nonproteinaceous polymers, typically a hydrophilic synthetic polymer, e.g., polyethylene glycol (PEG), polypropylene glycol, or polyoxyalkylene, as described in, e.g., U.S. Pat. Nos. 4,179,337, 4,301,144, 4,496,689, 4,640,835, 4,670,417, and 4,791,192, or a similar polymer such as polyvinylalcohol or polyvinylpyrrolidone (PVP). Mimetics of the fusion proteins are also contemplated. Suitable types of peptide mimetics are described in, e.g., U.S. Pat. No. 5,668,110 and the references cited therein. Furthermore, the fusion protein can be modified by the addition of protecting groups to the side chains of one or more the amino acids of the fusion protein. Such protecting groups can facilitate transport of the fusion peptide through membranes, if desired, or through certain tissues, for example, by reducing the hydrophilicity and increasing the lipophilicity of the peptide. Examples of suitable protecting groups include ester protecting groups, amine protecting groups, acyl protecting groups, and carboxylic acid protecting groups, which are known in the art (see, e.g., U.S. Pat. No. 6,121,236). Synthetic fusion proteins of the invention can take any suitable form. For example, the fusion protein can be structurally modified from its naturally occurring configuration to form a cyclic peptide or other structurally modified peptide.

The second peptide portion is preferably derived from, based upon, or obtained from a soluble protein, comprises a soluble portion of an otherwise insoluble protein, or is rendered soluble upon, or shortly after, administration or expression (e.g., by partial enzymatic cleavage or controlled degradation), thereby promoting the extracellular matrix mobility of the fusion protein. The fusion protein preferably is capable of relatively high diffusion mobility in the extracellular matrix of a mammalian host. Desirably, for example, the fusion protein diffuses through the extracellular matrix in a mammalian host upon administration to a mammalian host from a point of administration, the cell in which a polynucleotide encoding the fusion protein is expressed, or both, farther than a protein consisting essentially of a naturally occurring heparin-binding form of a VEGF (e.g., farther than a VEGF₁₈₉ or VEGF₂₀₆ administered under substantially identical conditions, more preferably farther than a VEGF₁₆₅ administered under substantially identical conditions). Migration/diffusion of the fusion protein can be detected by any suitable technique (e.g., radioactive or fluorescent antibody binding and detection assays or direct fluorescent staining detection).

The fusion protein alternatively, or preferably additionally, will diffuse in the extracellular matrix in a mammalian host upon administration to a mammalian host from a point of administration, the cell in which a polynucleotide encoding is expressed, or both, farther than a protein consisting essentially of the second peptide portion. For example, fusion proteins of the invention where the second peptide portion comprises a peptide that, in its native state, is associated with a high order of multimerization but are modified in the fusion protein to only form lower level multimers (e.g., tetramers, trimers, or dimers), for example, can exhibit significant improvements in mobility. The higher half-life associated with the fusion proteins of the invention (as further discussed herein) also permits longer range of diffusion, and, accordingly, reduces the number of doses of fusion protein (or vector containing a polynucleotide encoding the fusion protein) required for therapeutic administration.

As indicated above, the fusion proteins of the invention exhibit improved in vivo half-life over known angiogenic peptides and fusion proteins. For example, the fusion proteins of the invention typically will have a half life in a mammalian host at least twice as long (preferably at least three times as long, and more preferably at least five times as long) than the half life of a protein consisting essentially of an Ang-1. Typically, the fusion proteins will exhibit a half-life of at least three minutes, desirably at least about four minutes, more preferably at least five minutes, and even more preferably at least ten minutes (e.g., at least about 15, 20, 30, 60, 90, 180, 360, or 720 minutes) in a mammalian host upon administration (including direct administration as well as production upon expression of polynucleotides encoding the fusion proteins). The extended half-life is typically associated with the structure of the fusion protein, i.e., the combination of the VEGF peptide portion and second peptide portion where one or more domains of the second peptide portion (e.g., the Ang-1 coiled coil domain) or VEGF peptide portion which are associated with short in vivo half life are deleted or modified. Preferably, the fusion protein retains at least the eight cysteine residues conserved among the VEGFs, as previously mentioned, and more preferably, comprises even more cysteine residues in the second peptide portion, thereby rendering the fusion protein more resistant to extracellular degradation than other therapeutic factors (e.g., PDGFs). Wound healing fusion proteins including a CTGF second peptide portion are particularly preferred in this respect. Even longer half-life can be obtained, if desired, by fusion with a heterologous peptide portion which exhibits a longer in vivo half life (e.g., an IgG domain) (as described in, e.g., International Patent Application WO 00/24782), or by administering the fusion protein with a non-proteinaceous polymer, such as those described elsewhere herein.

The invention further provides polynucleotides including at least one nucleotide sequence which, when expressed in a cell permissive for expression of the nucleotide sequence, result in the production of the fusion protein. The polynucleotide sequence can be any suitable sequence (e.g., single stranded or double stranded RNA, DNA, or combinations thereof) and can include any suitable nucleotide base, base analog, and/or backbone (e.g., a backbone formed by, or including, a phosphothioate, rather than phosphodiester, linkage). Examples of suitable modified nucleotides which can be incorporated in the polynucleotide sequence are provided in the Manual of Patent Examining Procedure § 2422 (7th Revision—2000). The polynucleotide sequence can be any suitable length (e.g., about 100 nt, about 1000 nt, about 2500 nt, about 5000 nt, or even larger). The polynucleotide sequence can be any sequence that results in the fusion protein being produced upon expression, and, thus, is not limited to sequences which directly code for expression of the fusion protein. For example, the polynucleotide can comprise a sequence which results in the fusion protein through intein-like expression (as described in, e.g., Colson and Davis, Mol. Microbiol., 12(3), 959-63 (1994), Duan et al., Cell, 89(4), 555-64 (1997), Perler, Cell, 92(1), 1-4 (1998), Evans et al., Biopolymers 51(5), 333-42 (1999), and de Grey, Trends Biotechnol., 18(9), 394-99 (2000)), or a sequence which contains self-splicing introns which form the peptide portions and/or the fusion protein (as described in, e.g., U.S. Pat. No. 6,010,884). The polynucleotides also can comprise sequences which result in splice modifications at the RNA level to produce an mRNA transcript encoding the fusion protein and/or at the DNA level by way of trans-splicing mechanisms prior to transcription (as described in, e.g., Chabot, Trends Genet., 12(11), 472-78 (1996), Cooper, Am. J. Hum. Genet., 61(2), 259-66 (1997), and Hertel et al., Curr. Opin. Cell. Biol., 9(3), 350-57 (1997)). RNA based vectors may include removal of regions which promote degradation in the absence of hypoxia, e.g., by removal of the VEGF mRNA 3′ and/or 5′ UTRs (see Dibbens et al., Mol. Biol. Cell., 10, 907-19 (1999)) or portion thereof, e.g., the AU rich hairpin structure region of the 3′ UTR (see, e.g., Pages et al., J. Biol. Chem. (published on Jun. 9, 2000 as manuscript M002104200—American Society for Biochemistry and Molecular Biology, Inc.), and Levy, J. Biol. Chem., 271, 25492-25497 and 2746-2753 (1996)), particularly where RNA vectors are administered in the absence of hypoxic conditions. The polynucleotide can comprise one or more sequences encoding fusion proteins wherein the fusion protein-encoding sequence is codon optimized for a particular species (e.g., humans) (using techniques such as those described in U.S. Pat. Nos. 5,082,767, 5,786,464, and 6,114,148). For example, the second peptide portion can comprise a codon optimized mouse angiopoietin.

Preferably, in addition to the nucleic acid sequence which, when expressed, results in the fusion protein (the “fusion protein nucleic acid sequence”), the polynucleotide further includes one or more suitable “expression control sequences” operably linked to the sequence encoding the fusion protein. An expression control sequence is any nucleotide sequence that assists or modifies the expression (e.g., the transcription, translation, or both) of the nucleic acid encoding the angiogenic sequence. The expression control sequence can be naturally associated with the VEGF peptide portion or second peptide portion (e.g., a wild-type VEGF promoter), or can comprise a heterologous element with respect to the both the VEGF and second peptide portion polynucleotides. For example, the fusion protein nucleic acid sequence can be operably linked to a constitutive promoter (e.g., the Rous sarcoma virus long terminal repeat (RSV LTR) promoter/enhancer or the cytomegalovirus major immediate early gene (CMV IE) promoter, which is particularly preferred), an inducible promoter, (e.g., a growth hormone promoter, metallothionein promoter, heat shock protein promoter, E1B promoter, hypoxia induced promoter, or MLP promoter and tripartite leader), an inducible-repressible promoter, a developmental stage-related promoter (e.g., a globin gene promoter), or a tissue specific promoter (e.g., a smooth muscle cell α-actin promoter, VEGF receptor promoter, myosin light-chain 1A promoter, or vascular endothelial cadherin promoter). In some instances, host-native promoters can be preferred over non-native promoters (e.g., a human beta actin promoter or EF1α promoter driving expression of the fusion protein nucleic acid sequence can be preferred in a human host), particularly where strict avoidance of gene expression silencing due to host immunological reactions is desirable. The polynucleotide can include expression control sequences wherein one or more regulatory elements have been deleted, modified, or inactivated. the polynucleotide also or alternatively can include a bidirectional promoter system (as described in e.g., U.S. Pat. No. 5,017,478) linked to multiple genes of interest (e.g., multiple fusion protein encoding genes). The polynucleotide can further comprise site-specific recombination sites, which can be used to modulate transcription of the polynucleotide, as described in, e.g., U.S. Pat. Nos. 5,801,030 and 6,063,627 and International Patent Application WO 97/09439.

The polynucleotide can include or consist of any suitable fusion protein nucleic acid sequence. Preferred fusion protein nucleic acid sequences include nucleotide sequences which, when expressed, result in the production of the above-described fusion proteins (e.g., a polynucleotide comprising a sequence encoding a VEGF121 fused to a polynucleotide encoding an Ang-1 peptide portion, an aFGF peptide portion, a HBNF peptide portion, an MK peptide portion, an alkaline phosphatase peptide portion, or a fragment thereof which promotes angiogenesis, bone growth, or wound healing, or associated with such a second peptide portion-encoding sequence through a polynucleotide encoding a linker sequence, a sequence which does not effect production of the fusion protein upon expression (e.g., a sequence coding for intein-like expression), or other transcriptionally inert sequence (e.g., an intron). The polynucleotide can contain any suitable number of copies of the fusion protein nucleic acid sequence.

Preferably, the polynucleotide comprises a second nucleotide sequence that, when expressed, produces a second protein which promotes angiogenesis, bone growth, wound healing, or any combination thereof. The second nucleotide sequence can thus encode, for example, a second fusion protein or one of the angiogenic, bone growth promoting, or wound healing promoting factors described above (including their homologs and gene fragments thereof). The second nucleotide sequence also can encode a receptor for either the VEGF peptide portion or second peptide portion of the fusion protein, or for another encoded factor. In this respect, the polynucleotide can include any suitable number of protein-encoding sequences. Alternatively, the polynucleotide can encode for a ribozyme or for the production of an inhibitory (e.g., antisense) polynucleotide, which preferably facilitates one of the above-mentioned biological activities through inhibition of a biological activity inhibitor.

If the polynucleotide encodes multiple gene products, a combination of expression control sequences (e.g., promoters) can be used, preferably which correspond to a pre-planned pattern of activity with the desired pattern and level of expression of the encoded factors. Thus, nucleotide sequences in the polynucleotide can be under the control of separate promoters having different expression profiles, e.g., at least one nucleic acid sequence is operably linked to an RSV promoter and at least one other nucleic acid sequence is operably linked to a CMV promoter. Alternatively, a hybrid promoter can be constructed which combines the desirable aspects of multiple promoters. For example, a CMV-RSV hybrid promoter combining the CMV promoter's initial rush of activity with the RSV promoter's high maintenance level of activity is especially preferred for use in many embodiments of the inventive method. Thus, the invention provides polynucleotides where the fusion protein sequence is operably linked to a first promoter and a second nucleotide sequence is operably linked to a second promoter, such that the initiation of expression of the first nucleotide sequence and second nucleotide occurs at different times, in response to different factors, or both. Preferably, such promoter systems are designed to mimic expression patterns associated with normal biological activities, e.g., pathways or cascades. For example, a first promoter can drive the early expression (or separately inducible expression) of a first fusion protein which contains a VEGF peptide portion and an extracellular matrix degrading second peptide portion, and a second promoter can be later induced or otherwise later cause expression of a nucleic acid sequence encoding a factor that induces blood vessel remodeling, induces maturation, and/or reduces plasma leakage.

The polynucleotide can include multiple fusion protein genes and/or related genes to be serially and/or co-expressed. Thus, for example, the invention contemplates administration of polynucleotides which encode at least 3, at least 4, at least 5, or more, fusion protein genes or combinations of fusion protein and other angiogenic-bone growth promoting-, or wound healing promoting-factor encoding genes, which preferably mimic an expression pattern of a normal biological cascade. For example, a polynucleotide, which provides for sequential expression of (1) an MMP or TIMP (which provides matrix degradation), (2) an angiogenic VEGF fusion protein (which preferably attracts endothelial cells and induces blood vessel formation), (3) a vascular maturation factor (e.g., an Ang-1, ARF, or related fusion protein), and (4) a stabilization and maintenance factor (e.g., an ephrin), can be administered to the host (preferably in an ischemic tissue) to mimic the normal cascade of factors associated with blood vessel development. Administration of polynucleotides that express both growth and wound healing promoting factors in such a cascade-like fashion also are provided. Alternatively, multiple polynucleotides (e.g., within multiple vectors) can be administered, wherein the polynucleotides encode one or multiple genes to provide such a cascade effect. However, the administration of a single polynucleotide under control of the above-described expression control sequence systems is preferred.

Production of the recombinant polynucleotide encoding the fusion protein can be accomplished by any suitable technique. Recombinant polynucleotide production is well understood, and methods of producing such molecules are provided in, e.g., Ibanez et al., EMBO J., 10, 2105-10 (1991), Ibanez et al., Cell, 69, 329-41 (1992), and U.S. Pat. Nos. 4,440,859, 4,530,901, 4,582,800, 4,677,063, 4,678,751, 4,704,362, 4,710,463, 4,757,006, 4,766,075, and 4,810,648, and are more particularly described in Sambrook and Ausubel, supra.

The polynucleotide is preferably positioned in and/or administered in the form of a suitable delivery vehicle (i.e., a vector). The vector can be any suitable vector. For example, the nucleic acid can be administered as a naked DNA or RNA vector (including, for example, a linear expression element or a plasmid vector such as pBR322, pUC 19/18, or pUC 118/119) or as a precipitated nucleic acid vector construct (e.g., a CaPO₄ precipitated construct). The vector also can be a shuttle vector, able to replicate and/or be expressed (desirably both) in both eukaryotic and prokaryotic hosts (e.g., a vector comprising an origin of replication recognized in both eukaryotes and prokaryotes). The nucleic acid vectors of the invention can be associated with salts, carriers (e.g., PEG), formulations which aid in transfection (e.g., sodium phosphate salts, Dextran carriers, iron oxide carriers, or gold bead carriers), and/or other pharmaceutically acceptable carriers, some of which are described herein. Alternatively or additionally, the polynucleotide vector can be associated with one or more transfection-facilitating molecules such as a liposome (preferably a cationic liposome), a transfection facilitating peptide or protein-complex (e.g., a poly(ethylenimine), polylysine, or viral protein-nucleic acid complex), a virosome, a modified cell or cell-like structure (e.g., a fusion cell), or a viral vector.

More preferably, the polynucleotide is positioned in, and administered to the host via, a viral vector. The viral vector can be any suitable viral vector. A viral vector in the context of the invention includes any combination of nucleotides and proteins which are derived from, obtained from, or based upon proteins and or nucleic acids that are present in a wild-type virus. The viral vector can be a vector which requires the presence of another vector or wild-type virus for replication and/or expression (i.e., a helper-dependent virus), such as an adenoviral vector amplicon. The viral vector preferably consists of an intact virus particle. Typically, such viral vectors consist essentially of a wild-type viral particle, or a viral particle modified in its protein and/or nucleic acid content to increase transgene capacity or aid in transfection and/or expression of the nucleic acid (examples of such vectors include the herpes virus/AAV amplicons). Such vectors are typically named for the type of virus they are obtained from, derived from, or based upon, as applicable. Examples of preferred viral vectors include herpes viral vectors, adeno-associated viral vectors, and adenoviral vectors.

The construction of recombinant viral vectors is well understood in the art. For example, adenoviral vectors can be constructed and/or purified using the methods set forth, for example, in U.S. Pat. Nos. 5,965,358 and 6,168,941 and International Patent Applications WO 98/56937, WO 99/15686, WO 99/54441, and WO 00/32754. Adeno-associated viral vectors can be constructed and/or purified using the methods set forth, for example, in U.S. Pat. No. 4,797,368 and Laughlin et al., Gene, 23, 65-73 (1983). Similar techniques are known in the art with respect to other viral vectors, particularly with respect to herpes viral vectors, lentiviral vectors, and other retroviral vectors.

Desirably, the viral vector is capable of expressing the polynucleotide for a sustained period (e.g., for a period of at least about 1 day, preferably about 1 week), without expressing the polynucleotide so long that undesired effects associated with prolonged expression, e.g., promiscuous angiogenesis, occurs (e.g., for a period of less than about 2 weeks). Thus, the viral vector preferably is capable of therapeutic, and transient, self-terminating expression of the polynucleotide (e.g., expression for a period of about 1 week or less). Preferably, the viral vector achieves gene transfer in both dividing and non-dividing, as well as terminally differentiated, cells, with high levels of expression in cardiovascular relevant sites such as the myocardium, vascular endothelium, and skeletal muscle. The viral vector desirably is safe for administration to the host. Advantageously, the viral vector operates in an epichromosomal manner without insertion of genetic material to the host. Adenoviral vectors, which possess all of these aforementioned qualities, are particularly preferred delivery vectors for nucleic acid angiogenic mediators.

Any suitable adenoviral vector can be used as a delivery vehicle for the polynucleotide. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Preferably, the adenoviral vector is based on, derived from, or consists of a serotype-2 or serotype-5 adenovirus.

Regions of the adenoviral genome (e.g., the E3 region) in the adenoviral vector can optionally and preferably be deleted in order to provide space for insertion of the polynucleotide or other nucleic acid sequences. In addition, regions of the adenoviral genome can be deleted or altered in order to interfere with viral replication. The adenoviral vector used in the inventive method is preferably deficient in at least one gene function required for viral replication, thereby resulting in a “replication-deficient” adenoviral vector. Preferably the adenoviral vector will be deficient in at least one essential gene function of the E1, E2, and/or E4 regions of the adenoviral genome. More preferably, the adenoviral vector is deficient in at least one essential gene function of the E1 region (e.g., deficient in at least part of the E1aa region and/or at least part of the E1b region) of the adenoviral genome. Other portions of the genome also can be deleted, e.g., typically the E3 region, which is non-essential for viral replication. Thus, the adenoviral vector can be lacking multiple adenoviral gene functions, e.g., at least one essential gene function of the E1 region and at least one essential gene function of the E4 region, in addition to at least part of the E3 region. Examples of E1-deleted and other replication deficient adenoviral vectors are disclosed in, for example, U.S. Pat. Nos. 5,851,806 and 5,994,106 and International Patent Applications WO 95/34671 and WO 97/21826. The adenoviral vector desirably retains at least one adenovirus inverted terminal repeat (ITR) (preferably the 5′ and 3′ ITRS). The adenoviral vector also desirably retains the adenovirus packaging sequence. Preferably, the recombinant adenovirus also comprises a mutation in the major late promoter (MLP), as discussed in International Patent Application WO 00/00628.

A particularly preferred adenoviral vector for use in the inventive method is deficient in the entire E1a region, at least part of the E1b region, and at least part of the E3 region of the adenoviral genome and contains a DNA encoding a VEGF₁₂₁:Ang-1 fusion protein under the control of the CMV IE promoter in the E1 region of the adenoviral genome. Such a vector supports in vivo expression of the fusion protein that is maximized at one day following administration and is not detectable above baseline levels as little as one week after administration. This is ideal inasmuch as it is sufficient to provide substantial growth of new vasculature while minimizing adverse neovascularization at distal sites. In that regard, when this vector is locally administered to a target tissue, no detectable expression of the fusion protein can be detected in blood serum using standard ELISA monitoring assays. Advantageously, local administration to a target tissue of such adenoviral vectors including the polynucleotide encoding the fusion protein positioned in the E1 region of the adenoviral genome results in an at least 3-fold increase in blood flow in the extremities of mammals (e.g., the hind limb of Sprague-Dawley rats) with iliac and femoral artery ligations.

The adenoviral vector can be subject to any number of additional or alternative modifications. For example, a particularly preferred vector comprises a replication deficient adenoviral vector which includes or expresses a modified adenoviral protein, non-adenoviral protein, or both, which increases the efficiency that the vector infects cells as compared to wild-type adenovirus, allows the vector to infect cells which are not normally infected by wild-type adenovirus, results in a reduced host immune response in a mammalian host as compared to wild-type adenovirus, or any combination thereof. Any suitable type of modification can be made to the vector, and several suitable modifications are known in the art. For example, the adenoviral vector coat protein can be modified. Examples of such modifications include modifying the adenoviral fiber, penton, pIX, pIIIa, or hexon proteins, and/or insertions of various native or non-native ligands into portions of such coat proteins. Manipulation of such coat proteins can broaden the range of cells infected by a viral vector or enable targeting of a viral vector to a specific cell type. One direct result of manipulation of the viral coat is that the adenovirus can bind to and enter a broader range of eukaryotic cells than a wild-type virus. Examples of adenoviruses including such modifications are described in International Patent Application WO 97/20051. Reduction of immune response against the adenoviral also or alternatively can be obtained through the methods described in U.S. Pat. No. 6,093,699. In other embodiments, the viral coat is manipulated such that the virus is “targeted” to a particular cell type, e.g., those cells expressing unique receptors. Examples of such modified adenoviral vectors are described in U.S. Pat. Nos. 5,559,099, 5,731,190, 5,712,136, 5,770,442, 5,846,782, 5,962,311, 5,965,541, and 6,057,155 and International Patent Applications WO 96/07734, WO 96/26281, WO 97/20051, WO 98/07865, WO 98/07877, WO 98/40509, WO 98/54346, and WO 00/15823. Other adenoviral vector protein modifications that decrease the potential for immunological recognition by the host and resultant coat-protein directed neutralizing antibody production, as described in, e.g., International Patent Applications WO 98/40509 and WO 00/34496. In non-viral vector systems, the use of targeting through targeted proteins (e.g., an asialoorosomucoide protein conjugate which promotes liver targeting (such as is described in Wu and Wu, J. Biol. Chem., 263 (29), 14621-24 (1988)) or the targeted cationic lipid compositions of U.S. Patent 6,120,799).

The adenoviral vector also can include a trans-acting factor, cis-acting factor, or both, which preferably increases the persistence of transgene expression from the adenoviral vector's genome. Any suitable trans-acting factor can be used, such as HSV ICP0, which prolongs transgene expression (e.g., expression of the fusion protein sequence). Such modifications are particularly preferred in E4-deleted adenoviral vectors. The use of transacting factors is further described in International Patent Application WO 00/34496. Additionally or alternatively, the adenoviral vector comprises a nucleic acid sequence encoding a cis-acting factor. For example, a matrix attachment region (MAR) sequence (e.g., an immunoglobulin heavy chain μ (as discussed in, e.g., Jenuwein et al., Nature, 385(16), 269 (1997)), locus control region (LCR) sequences, or apolipoprotein B sequence (as discussed in, e.g., Kalos et al., Molec. Cell. Biol., 15(1) 198-207 (1995)) can be used to modify the persistence of expression from a transgene, such as a transgene inserted into an E4-deleted region of the adenoviral vector genome. LCR sequences are also believed to establish and/or maintain transcription of transgenes in a cis manner.

The polynucleotide can be positioned within any suitable location in the genome of the adenoviral vector. Typically, the polynucleotide will substitute for one or more of the aforementioned deleted regions of the adenoviral genome (e.g., the E1, E2, E3, and/or E4 region, most preferably replacing at least a portion of the E1 region). Alternatively, several polynucleotides encoding multiple fusion proteins, or fusion proteins and other proteins (e.g., a second angiogenic, bone growth promoting, or wound healing promoting peptide) can be inserted as expression cassettes into multiple deleted regions (e.g., a first angiogenic sequence can be inserted in a portion of the E1 region and the polynucleotide encoding the fusion protein can be inserted in the deleted E3 region, or vice versa).

Production of such deficient adenoviral vectors can be accomplished by use of a complementation cell line, which is capable of providing the deleted necessary adenoviral gene functions in trans. Several examples of suitable cells are known. Examples of suitable cells for producing such vectors include 293 cells (described in, e.g., Graham et al., J. Gen. Virol., 36, 59-72 (1977)), PER.C6 cells (described in, e.g., U.S. Pat. No. 5,994,128), 911 cells (as described in, e.g., Fallaux et al., Human Gene Therapy, 7, 215-222 (1996)), and 293-ORF6 cells (as described in, e.g., International Patent Application WO 95/34671 and Brough et al., J. Virol., 71, 9206-13 (1997)). The cell line can provide either no homologous overlapping regions with the adenoviral vector, ideally resulting in no replication competent adenovirus (RCA), or, alternatively can partially overlap in one or more essential regions but lack homology in one or more essential regions (as exemplified by the cells in International Patent Application WO 95/34671). Desirably, the vector composition of the invention is formed from a purified stock of such vectors. A preferred method for purifying such vector stocks is provided in International Patent Application WO 99/54441. Methods for assessing the purity of such vector compositions are provided in International Patent Application WO 00/12765.

The polynucleotide encoding the fusion protein can be inserted in any of the above-described vectors in any suitable manner and in any suitable orientation. Whereas the polynucleotide can be inserted in any suitable orientation, preferably the orientation of the nucleic acid is from right to left. By the polynucleotide having an orientation “from right to left,” it is meant that the direction of transcription of the nucleic acid is opposite that of the region of the vector into which the polynucleotide is inserted.

The invention further provides methods of promoting angiogenesis, bone growth, wound healing, or any combination thereof in an individual (e.g., a mammalian host, such as a human) by administering to the individual the fusion protein, preferably in an amount effective to promote angiogenesis, bone growth, wound healing, or any combination thereof. Administration can be performed by any suitable method, and the fusion protein can be administered in any suitable form (including by way of the polynucleotide or vector described herein). Preferably, the fusion protein (or polynucleotide or vector encoding the fusion protein) is administered in a composition, with a carrier, preferably in a pharmaceutically acceptable composition, e.g. by combination with a pharmaceutically acceptable carrier.

The term “pharmaceutically acceptable” means that the composition is a non-toxic material that does not interfere with the effectiveness of the biological activity of the fusion protein or other effective ingredients. Any suitable carrier can be used, and several carriers for administration of therapeutic proteins are known in the art. The characteristics of the carrier will depend on the route of administration.

The pharmaceutical composition and/or pharmaceutically acceptable carrier also can include diluents, fillers, salts, buffers, stabilizers, solubilizers, and/or other materials suitable for inclusion in a pharmaceutically composition. The pharmaceutical composition of the invention also can contain preservatives, antioxidants, or other additives known to those of skill in the art. When the fusion protein (or polynucleotide or vector encoding is fusion protein) is administered with other agents or ingredients the combined amounts of the agents can be administered in combination, serially or simultaneously.

The pharmaceutical composition of the invention can be in the form of a liposome in which the fusion protein (or polynucleotide or vector encoding the fusion protein) is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is described in, e.g., U.S. Pat. Nos. 4,837,028 and 4,737,323.

The pharmaceutical composition can be delivered to the individual by any suitable route of administration. Examples of suitable routes of administration include oral ingestion, inhalation, bucal application, rectal application, vaginal application, topical application, insufflation, implantation, transmucosal administration, or cutaneous, subcutaneous, intraperitoneal, parenteral, myocardial, pericardial (e.g., intrapericardial), or injection (e.g., intravenous injection). Intravenous administration and injection are preferred.

If the pharmaceutical composition is administered orally, the composition preferably is administered in the form of a tablet, capsule, powder, solution, elixir, or troches. Oral compositions can include any suitable carriers or other agents. For example, tablets will typically contain a solid carrier, such as a gelatin. Generally, oral compositions also can include binders (e.g., microcrystalline cellulose, gum tragacanth or gelatin), excipients (e.g., starch or lactose), disintegrating agents (e.g., alginic acid, Primogel, or corn starch), lubricants (e.g., magnesium stearate or Sterotes), glidants (e.g., colloidal silicon dioxide), and/or sweetening/flavoring agents. Oral compositions preferably contain about 5-95%, preferably about 25-90%, fusion protein (or polynucleotide or vector encoding the fusion protein).

To administer the fusion protein (or polynucleotide or vector encoding the fusion protein) in a liquid form, such as in delivery by injection, a liquid carrier such as water, petroleum, physiological saline, bacteriostatic water, Cremophor ELTM (BASF, Parsippany, N.J.), phosphate buffered saline (PBS), or oils can be used as a carrier. Liquid pharmaceutical compositions can further contain physiological saline solution, dextrose or other saccharide solution, or glycols, such as ethylene glycol, propylene glycol, PEG, coating agents which promote proper fluidity, such as lecithin, isotonic agents, such as manitol or sorbital, and absorption-delaying agents, such as aluminum monostearate. When administered in liquid form, the pharmaceutical composition preferably contains about 0.5-90% by weight (wt.%) of fusion protein (or polynucleotide or vector encoding the fusion protein), more preferably about 1-50 wt.% fusion protein (or polynucleotide or vector encoding the fusion protein).

More particularly, when the pharmaceutical composition is administered by injection, the composition will preferably be in the form of a pyrogen-free, stable, parenterally acceptable aqueous solution. Preferably, the parenterally acceptable aqueous solution comprises an isotonic vehicle such as sodium chloride injection, Ringer's injection, dextrose injection, lactated Ringer's injection, or equivalent delivery vehicle (e.g., sodium chloride/dextrose injection).

In a particularly preferred aspect, the fusion protein (or polynucleotide or vector encoding the fusion protein) are administered in or near the heart. Administration in or near the heart can be to any suitable heart-associated region or tissue, using any suitable technique. Examples of suitable types of administration include direct (needle or biolistic) intracoronary injection (e.g., of a vector composition) and/or intracoronary administration using implant devices (e.g., a fusion protein coated coronary stent). Pericardial, myocardial, and intracoronary administration are particularly preferred for angiogenic fusion proteins used to treat vascular occlusion in an individual's heart.

For compositions to be administered to bone, cartilage, tendon, or ligaments (e.g., for promoting bone growth or wound healing), the therapeutic method includes administering the composition, systematically or locally as an implant or device, desirably in a pyrogen-free, physiologically acceptable form. Further, the composition can desirably be encapsulated or injected in a viscous form for delivery to the site of bone, cartilage, or tissue damage. Bone and/or cartilage formations also or alternatively can include a matrix capable of delivering the fusion protein-containing composition to the bone and/or cartilage administration site, providing a structure for the developing bone and cartilage. Advantageously, the matrix is capable of being resorbed into the body. Suitable materials for producing such matrixes include calcium sulfate, tricalciumphosphate, hydroxyapatite, polylactic acid, polyglycolic acid, bone and/or dermal collagens, and polyanhydrides. Additional suitable administration techniques and matrixes are discussed elsewhere herein.

Topical administration also can be suitable for wound healing and tissue repair. For example, a drug reservoir or monolithic matrix transdermal patch device can be used for such topical administration, as can creams, ointments, or salves.

Administration devices can be formed of any suitable material. Examples of suitable matrix materials for producing non-biodegradable administration devices include hydroxapatite, bioglass, aluminates, or other ceramics. In some applications, a sequestering agent, such as carboxymethylcellulose (CMC), methylcellulose, hydroxypropylmethylcellulose (HPMC), or autologous blood clot, can be used to prevent the fusion protein complex from disassociating from the device and/or matrix. Thus, such sequestering agents are preferably present in an amount which prevents desorption of the fusion protein from the matrix/device and/or provides better handling of the composition. Typically, such sequestering agents will make up about 0.5-20 wt. %, preferably 1-10 wt. %, of the composition, based on total formulation weight.

For administration by inhalation, the fusion protein (or polynucleotide or vector encoding the fusion protein) can be delivered in the form of an aerosol spray from pressured container or dispenser which contains a suitable propellant, e. g., a gas such as carbon dioxide, or a nebulizer.

For transmucosal or transdermal administration, penetrants appropriate to the barrier to be permeated are preferably included in the composition. Such penetrants are generally known in the art, and include, for example, for transmucosal administration, detergents, bile salts, and fusidic acid derivatives. Transmucosal administration can be facilitated through the use of nasal sprays or suppositories.

The invention further provides sterile compositions, such as sterile powder compositions, that comprise the fusion protein (or polynucleotide or vector encoding the fusion protein), e.g., for the preparation of sterile injectable solutions. Such powder compositions can be prepared by, e.g., vacuum drying and freeze-drying, which yields a powder of the active ingredient plus any additional desired ingredient from a previously sterile-filtered solution thereof. The compositions of the invention also or alternatively can be in provided in unit dose containers and devices, including ampoules, disposable syringes, or multiple dose vials.

Additional pharmaceutically acceptable carriers are known in the art. Examples of additional suitable carriers are described in, e.g., Urquhart et al., Lancet, 16, 367 (1980), Lieberman et al., Pharmaceutical Dosage Forms—Disperse Systems (2nd ed., vol. 3, 1998), Ansel et al., Pharmaceutical Dosage Forms & Drug Delivery Systems (7th ed. 2000), Remington's Pharmaceutical Sciences, and U.S. Pat. Nos. 5,708,025 and 5,994,106.

The specific amount of fusion protein (or polynucleotide or vector encoding the fusion protein) in a dosage of the composition administered to the individual will depend upon the biological effect desired in the individual, condition to be treated, and/or the specific characteristics of the fusion protein (or polynucleotide or vector encoding the fusion protein) and individual. Preferably, the pharmaceutical composition is administered in a therapeutically effective amount. A “therapeutically effective amount” means an amount sufficient to show a meaningful benefit in an individual, i.e., promoting at least one aspect of angiogenesis, bone growth, wound healing, or combination thereof, or treatment, healing, prevention, or amelioration of other relevant medical condition(s). Therapeutically effective amounts may vary depending on factors such as those described above. Thus, the attending physician (or other medical professional responsible for administering the composition) will typically decide the amount of fusion protein with which to treat each individual patient. Generalized guidance in making such determinations can be found, for example, in Platt, Clin. Lab Med., 7, 289-99 (1987), and in “Drug Dosage,” J. Kans. Med. Soc., 70(1), 30-32 (1969).

Proper dosage can be determined by any suitable technique. In a simple dosage testing technique, low doses of the composition are administered to a test subject or system (e.g., an animal model, cell-free system, or whole cell assay system). Larger doses of the composition then can be administered until the desired therapeutic effect is obtained. For example, Doppler imaging can be used to detect blood flow and/or microscopy can be used to detect changes in blood vessel number or quality. Preferably, the dosage is within a range that includes the ED50, with low average toxicity. Such dosages are expected to typically contain about 0.01 mg-100 mg, preferably about 0.1-10 mg, more preferably about 0.1-1 mg, of fusion protein per kg body weight. Dosages with respect to vectors containing polynucleotides encoding the fusion protein are described elsewhere herein; however, it should be understood that the discussion provided with respect to dosage and administration of the fusion protein and of such vectors are considered interchangeable unless explicitly stated otherwise or clearly contradicted by the text.

A more specific discussion of dosage with respect to wound healing fusion proteins is now provided as an example intended to further illustrate the invention, and in no way is intended to limit the invention.

In general, the effective dose of a wound healing-promoting fusion protein is expected to vary depending on the wound to be treated (e.g., size and type of wound), and the particular qualities of the fusion protein (particularly the second peptide portion). Typically, a dosage of about 1 ng/ml-500 μg/ml, preferably about 75 ng/ml-200 μg/ml, and more preferably about 30-100 ng/ml, will be effective (e.g., in a 5 ml topical application). Alternatively, the wound healing-promoting fusion protein can be administered in a dose of about 0.001 μg/kg-10 mg/kg of body weight. For VEGF/PDGF fusion proteins, a dose of about 75 ng/ml-7.5 μg/ml is preferred (e.g., 500 ng/ml). For VEGF/EGF fusion proteins, a dose of about 1000-5000 ng/ml is expected to be effective. VEGF/aFGF fusion proteins are preferably administered in dosages of at least 50 μg total fusion protein.

Dosage will vary with wound size. Deeper and severe wounds typically require higher doses of the fusion protein. Chronic wounds also may require higher dosages for effective treatment. With respect to wound size, dosage can be expressed, for example, as an amount of fusion protein per volume of wound tissue. For example, a dose of about 1-10 μg fusion protein in a 50 μl carrier composition can be administered per 2-5 cm wound (which corresponds to about 0.1-1 μg fusion protein/cm² of wound area). More particularly, for example, for a VEGF/IL-1 fusion protein, a dose of about 0.1-0.5 μg/cm² is preferred. Wound surface area is the area defined by the perimeter of the wound and can be estimated by multiplying the length and width of the wound. More accurate measurement of wound surface area can be obtained by use of a planimeter (Houston Instruments).

The dosage with respect to an angiogenic fusion protein desirably reduces or avoids the negative side effects associated with high dosages of VEGFs, such as macular degeneration, rheumatoid pannus formation, progression of atherosclerosis or plaque rupture, diabetic proliferative retinopathy, and increasing tumor growth. Thus, the dose of the angiogenic fusion protein or vector composition preferably will be such that the dose does not result in such effects. Similarly, dosages of bone growth promoting fusion protein and vector compositions preferably will be at a level below which abnormal levels of ossification occur. Vector compositions comprising targeted vectors, particularly targeted adenoviral vectors, are particularly preferred in these respects.

The invention further provides a method of producing the fusion protein by introducing a vector containing a polynucleotide, which, when expressed, results in the production of a fusion protein of the invention, into a suitable cell, such that the nucleotide sequence is expressed and the fusion protein is produced. The vector can be introduced into a suitable host cell for purpose of producing the fusion protein, which is then substantially isolated, preferably purified, which can be administered to an individual as described above. Any cell permissive for the uptake and maintenance of the vector and expression of the polynucleotide can be suitable. Examples of suitable cells include bacterial cells, such as E. coli and mammalian cloned cells, such as HeLa cells, CHO cells, and VERO cells. Preferred cells and vectors (i.e., cell-vector systems) are described elsewhere herein. Transformation of such cells can be accomplished using techniques described herein or in Sambrook and Ausubel, supra. The fusion protein produced in the host cell can be identified and substantially isolated (preferably completely isolated) using standard techniques, including genetic selection, cell surface display, phage and virus display, ribosome display, fluorescence-based cell sorting, and agar plate screening (preferably combined with automated colony picking). Where fewer candidates need to be screened, more sensitive and faster techniques such as HPLC, mass spectrometry, gas chromatography, or chromogenic techniques can be applied.

Alternatively, and preferably, such a vector is administered to an individual (e.g., a mammalian host, such as a human), resulting in the in vivo expression of the fusion protein. In vivo administration of the fusion protein by way of such vectors offers several advantages over direct protein administration, including, e.g., avoidance of the first pass effect and other metabolically-related processing problems, providing intracellular production and processing, and providing sustained administration over a period of time, thereby resulting in less need for repeated administration events. The vector containing the fusion protein-encoding polynucleotide will preferably be administered to an area of the individual's body such that it induces angiogenesis, bone growth, wound healing, or combination thereof.

In the case of vectors containing angiogenic fusion protein-encoding polynucleotides, the vector is desirably administered near one or more angiogenically functional locations (source locations) and at least one angiogenically dysfunctional location (target location). Desirably, the vector (or fusion protein) composition is administered in a gradient forming manner, as described in International Patent Application PCT/US00/030750. The source location can be any location in the individual (e.g., tissue or organ), which has physiologically normal levels of blood perfusion, such as an area near or imbued with existing blood vessels (e.g., a non-ischemic area). The target location preferably is an actual or potentially angiogenically dysfunctional location, e.g., a location in the host that is either undergoing or is at risk of undergoing ischemia or any other condition wherein the growth of new, or extension of existing, blood vessels is desirable. Thus, the target location typically will be suffering from or be at risk of suffering from ischemic damage, which results when the tissue is deprived of an adequate supply of oxygenated blood. The interruption of the supply of oxygenated blood is often caused by a vascular occlusion. Such vascular occlusion can be caused by arteriosclerosis, trauma, surgical procedures, disease, and/or other indications. There are many ways to determine if a tissue is at risk of suffering ischemic damage from undesirable vascular occlusion including, e.g., ^{99m}Tc-sestamibi scanning, x-ray imaging, Doppler imaging, and MRI scanning. The target location also can comprise a tissue in which blood flow is attenuated by trauma, surgery, or other events. The alleviation of such attenuated blood supply, regardless of its origin, is contemplated by the invention. Thus, prevention or alleviation of damage from indications such as myocardial ischemia (particularly in patients suffering from insulin dependent diabetes), delayed wound healing, Buerger's disease, and stroke are contemplated.

Additionally, the planning of a surgical procedure can be predictive of the interruption of blood supply through a particular portion of a patient's vasculature. Prior treatment according to the method of the invention can substantially improve the desired outcome of these surgeries. In that case, treatment preferably occurs about one day to about six weeks before the surgery, and more preferably about two to about fourteen days prior to surgery. Other prophylactic uses of the vector also are contemplated.

The target and source locations can be in any suitable tissue susceptible to new blood vessel growth upon expression of a therapeutic amount of the angiogenic fusion protein. For example, the target and source locations can be located in a discrete organ such as the brain, heart, pancreas, limbs, or generalized areas of the body, such as a leg or a foot. Preferably, the target location and source location comprise portions of an organ system that includes at least two arteries (e.g., a heart which comprises at least three major arteries). In such aspects, the target location typically comprises at least a portion of an angiogenically dysfunctional artery in the system (e.g., an artery suffering from vascular occlusion), and some, if not all, of the angiogenically functional arteries in the system serve as source locations. In such aspects, the angiogenic mediator preferably is administered in a distribution between the target artery and the source arteries. Where the target location is an artery suffering from vascular occlusion, the method can comprise administration of the vector upstream, downstream, or to the occluded region of the artery (i.e., with respect to normal blood flow), or any combination thereof, as desired, preferably such that induced collateral blood vessel development bypasses the occluded region. “Tissue” in this sense is thus meant to include interstitial spaces associated with solid tissue. The source and target locations also can comprise cavities or extracellular fluid next to a tissue.

The polynucleotide or vector can be administered in the form of a composition, e.g., with or in any suitable acceptable carrier, preferably a pharmaceutically acceptable carrier, such as those described elsewhere herein. Additional pharmaceutically acceptable carriers particularly suitable for administration of vectors are described in, for example, International Patent Application WO 98/32859.

The desired dosage (i.e., total dosage to the host) of the vector composition is such that the amount of fusion protein produced by expression of the polynucleotide in the vector results in a therapeutic and/or prophylactic effect in the area where the vector is administered. The dosage will depend on the type of fusion protein to be produced. Because a wide range of suitable fusion proteins are provided by the invention, dosage is described generally, augmented by examples relating to specific vector compositions. It will be understood that this type of description is meant to further illustrate the invention without limiting it to any particular vector composition.

Desirably, for vectors containing polynucleotides encoding angiogenic fusion proteins, the vector dosage is such that induction of angiogenesis in non-targeted tissue is minimized, and that the generation of disorganized vasculature beds, loss of function in the affected tissue, and promiscuous angiogenesis, which can be associated with over dosage, are avoided. Thus, the volume of vector composition is preferably set such that very little or no nucleic acid sequences encoding the angiogenic fusion protein are carried by the blood, lymphatic drainage, or physical mechanisms (e.g. gravitational flow or osmotic flow) to non-target locations.

Dosages of the vector composition will vary depending on the vector used to deliver the fusion protein-encoding polynucleotide and administration technique. For example, angiogenic fusion protein-encoding naked polynucleotide vectors will typically be administered in an amount containing about 500-6000 μg of polynucleotide vector (e.g., plasmid or linear expression element) and more preferably about 1000-4000 μg of polynucleotide vector. Because a large number of such vectors are available for administration, dosage is further described herein with respect to adenoviral vectors. It should be understood that the description of such dosages is intended to illustrate this aspect of the invention, and thereby enable the skilled artisan to determine proper dosage using other vectors. Accordingly, the focus on adenoviral vector dosage is not intended to limit the scope of the invention.

The dosage of an adenoviral vector containing a fusion protein-encoding polynucleotide will be at least about 1×10⁶ pfu (e.g., 1×10⁶-1×10¹³ pfu) to an area near, at, or between the target and source locations. The dose preferably is at least about 1×10⁷ pfu (e.g., about 1×10⁷-1×10¹³ pfu), more preferably at least about 1×10⁸ pfu (e.g., about 1×10⁸-1×10¹¹ pfu), and most preferably at least about 1×10⁹ pfu (e.g., about 1×10⁹-1×10¹⁰ pfu). The dose typically is for a volume of targeted tissue of about 0.5-15 cm³, but can be for larger tissue volumes of up to 100 cm³ or even about 150 cm³. The dose desirably is administered via multiple applications, and, as such, is divided among the multiple applications. Thus, if the dose is administered via 10 administrations, each administration involves about 1×10⁵-1×10¹² pfu. Preferably, each application involves about 1×10⁶-1×10¹² pfu, more preferably about 1×10⁷-1×10¹⁰ pfu, and most preferably about 1×10⁸-1×10⁹ pfu. For purposes of considering the dose in terms of particle units (pu), also referred to as viral particles, it can be assumed that there are 100 particles/pfu (e.g., 1×10¹² pfu is equivalent to 1×10¹⁴ pu). In a single round of vector administration, using, for example, an adenoviral vector deleted of the entire E1 a region, part of the E1b region, and part of the E3 region of the adenoviral genome, wherein the vector comprises a nucleic acid encoding, e.g., a VEGF/KIAA0003-encoded peptide fusion protein under the control of a standard CMV immediate early promoter, about 10⁷-10¹³ pfu, preferably about 10⁹-10¹¹ pfu, are administered to the host (e.g., to a discrete organ containing the source and/or target locations) with an estimated volume of about 150 cm³. Under these conditions, a substantial level of VEGF/KIA0003 fusion protein production is achieved in the tissue of interest without producing detectable levels of fusion protein production in distal tissues.

The vector composition can be administered to the individual by any suitable technique, including those techniques described herein with respect to fusion protein-containing compositions or polynucleotides and vectors. Preferably, the vector is injected into the individual. Injection can be performed in any suitable tissue or body part (e.g., intravenously, myocardially, parenterally, intrathecally, intradermally, subdermally, or into the interstitial space of a tissue/organ (e.g., of a muscle tissue)). By the term “injecting,” it is meant that the vector containing solution is forcefully introduced into the target tissue. The vector composition can be microinjected, injected directly by a needle, or injected by biolistic injection. Injection can be performed using any suitable device, such as the device described in U.S. Pat. No. 5,846,225. Alternatively, the vector containing composition can be delivered by means of percutaneous administration, typically by use of a device, such as a catheter (e.g., inserted into the femoral artery) or by a stent coated with a suitable vector containing composition (e.g., which is placed in a suitable artery, such as a coronary artery).

The vector alternatively or additionally can be administered to any suitable surface, either internal or external, at or near the source and/or target locations. For example, with respect to directly injecting a vector containing a polynucleotide encoding an angiogenic fusion protein into cardiac tissue, it is contemplated that such an injection can be administered from any suitable surface of the heart (i.e., the angiogenic mediator can be administered endocardially, epicardially, and/or pericardially). Typically and preferably, cardiac administration will be to or in the left free ventricular wall of the heart which is easily accessible by minimally invasive thoracotomy. Alternatively, administration to other areas of the heart (e.g., the septum and/or right ventricle) can be accomplished by use of a catheter or other percutaneous delivery device. Such alternate techniques can be desired where the target location is positioned in the heart but away from the left free ventricular wall (e.g., where the target location is a vascular occlusion in the right coronary artery). For wounds at or near the skin surface, topical and/or transdermal administration of vectors containing polynucleotides encoding wound healing fusion proteins are often preferred routes of administration.

Vectors containing polynucleotides encoding bone growth-promoting fusion proteins can be administered in association with orthopedic implants, interfaces, and/or artificial joints, such as, surgical screws, pins, and the like. In preferred embodiments, the metal surface or surfaces of an implant or a portion thereof, such as a titanium surface, can be coated with a material that has an affinity for the vector composition, such as hydroxyl apatite in the case of polynucleotide vectors, and the coated metal is subsequently coated in the vector composition, prior to administration. For administration of vectors containing such polynucleotides, surgical pins or similar devices can be used to create a segmental defect (e.g., an about 0-10 mm, preferably about 0-5 mm defect) in the bone tissue wherein an implant material (preferably formed of a biodegradable matrix as discussed elsewhere herein or as described in U.S. Pat. Nos. 4,526,909, 4,563,489, 4,596,574, and 5,270,300), coated with the vector composition, is then administered, followed by closure of the defect.

Alternatively, where a fracture exists, such compositions can be similarly administered to the fracture site. Preferably, the target of the vector composition for expression of the bone growth promoting fusion protein will include such a fracture site, an area of weak bone, such as an area of bone effected by osteoporosis, or a bone cavity site that one wishes to fill with new bone tissue (e.g., a dental or periodontal surgical related cavity, birth defect related cavity, or osteosarcoma removal related cavity). Such vector compositions also can be administered by use of collagen sponges, preferably surrounded with clotted blood placed in the cavity or osteomy gap, or collagen matrixes, such as those described in U.S. Pat. Nos. 4,394,370, 4,526,909, 4,563,489, 4,596,574, 4,975,527, and 5,270,300, mineralized collagen compositions (as described in, e.g., U.S. Pat. No. 5,231,169), or collagen compositions commercially available through Norian Corp. (Mountain View, Calif.). For gaps or cavities (induced or natural) of about 2 mm or less, a fusion protein that increases the rate of bone growth can be suitable, whereas for a gap or cavity of about 5 mm or more administration of an osteotropic fusion protein associated with new bone growth is desired.

In the case of fractures or related injuries, devices which apply mechanical stress to the bone can assist in bone healing. In addition, electrical stimulation and distraction osteogenesis can be applied to assist in promoting bone growth. Related factors, such as other bone-growth related proteins, polynucleotides encoding such proteins, and/or combinations of fusion proteins provided by the invention, can be co-administered with the vector composition.

Dosage considerations for bone growth-related vector compositions will depend on the bone growth promoting fusion protein to be expressed in the host, delivery matrix or composition (if any), the amount of bone weight desired to be formed, the site of bone damage, the condition of the damaged bone, the patient's or animal's age, sex, and diet, the severity of any infection, the time of administration and any further clinical factors that may affect bone growth, such as serum levels of various factors and hormones. The suitable dosage regimen, therefore, will be readily determinable by one of skill in the art in light of the present disclosure, bearing in mind the individual circumstances. In treating humans and animals, progress can be monitored by periodic assessment of bone growth and/or repair, e.g., using x-rays. Bone growth promoting vector compositions, particularly for larger gaps (e.g., about 5 mm), preferably permit expression of the fusion protein for at least about 1 week, more preferably at least about 4 weeks, and even more preferably at least about 8 weeks (e.g., 6-10 weeks). When expression is required for shorter periods of time (e.g., about 2 weeks or less), adenoviral vectors are preferred delivery vehicles for bone cells (as described in, e.g., Mehara et al., J. Bone Miner. Res., 14(8), 1290-301 (1999) and Takayanagi et al., J. Clin. Invest., 104(2), 137-46 (1999), Baltzer et al., Knee Surg. Sports Traumatol. Arthrosc., 7(3), 197-202 (1999), Tanaka et al., J. Bone Miner. Res., 13(11), 1714-20 (1998), and Riew et al., Calcif. Tissue Int., 63(4), 357-60 (1998)), using, e.g., the pu/pfu dosages described above. However, for longer periods of expression, naked polynucleotide vectors can be preferred over adenoviral vectors for such methods. Retroviral vectors also can be suitable, particularly for expression of between 4-8 weeks (see, e.g., Mason et al., Gene Ther., 5(8), 1098-104 (1998), for discussion). If acceptable, permanent cellular transformation, e.g., by microinjection of a polynucleotide associated with integration sequences, biolistic delivery of such a polynucleotide, or lentiviral transformation also can be used.

Compositions containing vectors comprising polynucleotides encoding wound growth-promoting fusion proteins also will vary with respect to dosage depending upon a number of factors. Typically, the vector composition will be able to express amounts of fusion proteins corresponding to the above-described amounts administered in protein form. Preferably, for epidermal, intradermal, or subdermal wounds, administration through application of a topical composition, transdermal delivery (e.g., through a monolithic matrix transdermal patch), or biolistic delivery, is used, and preferably repeated after intervals varying between 1-7 days during a time period from about 1-120 days, depending on the healing process. Preferred formulations for wound healing vector compositions include HPMC and carboxymethyl cellulose preparations, PEG preparations, and matrixes, preferably which facilitate targeting of repair cells, delivery of the vector composition, and/or sustained administration of the vector composition. Examples of suitable matrixes include those described elsewhere herein and in U.S. Pat. No. 5,270,300. Matrixes can take the form of sponges, implants, tubes, telfa pads, bandages, pads, lyophilized components, gels, patches, powders or nanoparticles. In addition, matrixes can be designed to allow for sustained release of the polynucleotide or vector composition over prolonged periods of time. In certain embodiments of the invented method, the wound healing vector composition is administered in conjunction with a wound dressing. Alternatively, administration can be accomplished through microspheres, particularly for skin-associated wounds. Examples of suitable microspheres are provided in U.S. Pat. Nos. 5,264,207 and 6,086,863.

In other aspects, the wound healing-promoting vector composition can be administered with in situ tissue scaffolding to reduce scar healing and promote normal wound healing. Alternatively or additionally, the vector composition can be administered in association with an artificial skin, e.g., a skin manufactured from neonatal foreskin. Application of such compositions are particularly preferred in addressing burn wounds.

Adenoviral vectors can be used for short-term administration in wound healing, in dosages such as those described above. Where longer expression is desired, retroviral vectors (e.g., lentivirus vectors) or adeno-associated viral (AAV) vectors can be advantageously used (as described in, e.g., Buschacher et al., Blood, 5(8), 2499-504, Carter, Contrib. Microbiol., 4, 85-86 (2000), Smith-Arica, Curr. Cardiol. Rep., 3(1), 41-49 (2001), Taj, J. Biomed. Sci., 7(4), 279-91 (2000), Vigna et al., J. Gene Med., 2(5), 308-16 (2000), Klimatcheva et al., Front. Biosci., 4, D481-96 (1999), Lever et al., Biochem. Soc. Trans., 27(6), 841-47 (1999), Snyder, J. Gene Med., 1(3), 166-75 (1999), Gerich et al., Knee Surg. Sports Traumatol. Arthrosc., 5(2), 118-23 (1998), and During, Adv. Drug Deliv. Review, 27(1), 83-94 (1997), and U.S. Pat. Nos. 4,797,368 , 5,139,941 , 5,173,414, 5,614,404, 5,658,785, 5,858,775, and 5,994,136). Alternatively, polynucleotide vectors can be used, or host integrative techniques can be employed. Preferably, for polynucleotide vectors, a collagen matrix-based delivery system of targeted DNA vectors is utilized (as described in, e.g., Chandler et al., Mol. Ther., 2(2), 153-60 (2000)). Co-administration of the wound healing-promoting vector composition or fusion protein with related wound healing factors is contemplated, such as the wound healing-promoting factors described herein, or non-protein factors involved in wound healing (e.g., vitamin-E or zinc).

The fusion protein, polynucleotide, or vector can be administered by or in association with ex vivo delivery of cells, tissues, or organs. Therefore, for example, a target tissue can be removed, contacted with the vector composition, and then reimplanted into the host (e.g., using techniques described in or similar to those provided in Crystal et al., Cancer Chemother. Pharmacol., 43(Suppl.), S90-S99 (1999)). Ex vivo administration of an angiogenic fusion protein, or preferably angiogenic vector composition, to the target tissue also helps to minimize undesirable induction of angiogenesis in non-targeted tissue. A specific example of such a technique is the administration of an angiogenic vector composition to a tissue flap in surgical procedures involving replacement and/or transfer of tissue flaps (e.g., in breast reconstruction). “Tissue flaps” thus can comprise portions of removed tissue from a living tissue, a tissue of the recently deceased, a tissue from a different species (e.g., a pig tissue, preferably a tissue that is modified to exhibit a reduced immune response upon application to a human), or a synthetically generated tissue. Examples of suitable tissues are described in, e.g., U.S. Pat. No. 6,140,039. Cultures of cells, particularly three dimensional cultures, which can be a suitable substitute, additive, or alternative to such tissues also can be administered in association with the fusion protein, polynucleotide, or vector of the invention. Examples of suitable cultures in this respect are provided in U.S. Pat. Nos. 6,039,760, 6,022,743, 5,902,741, 5,863,531, 5,858,721, 5,849,588, 5,843,766, 5,830,708, 5,785,964, 5,624,840, 5,580,781, 5,578,485, 5,541,107, 5,518,915, 5,516,681, 5,516,680, 5,512,475, and 5,510,254. Related methods and compositions are provided in, e.g., U.S. Pat. Nos. 6,121,042, 6,060,306, 6,027,306, 6,008,049, 5,928,945, 5,842,477, 5,780,295, 5,714,588, and 5,559,022. Cells that are genetically transformed with the polynucleotides or a host genome integrative vector also can be administered in an ex vivo manner to the host (e.g., using the techniques described in, or similar to those described in, U.S. Pat. No. 5,399,346). For example, keratinocytes or fibroblasts can be cultured in vitro, transformed so as to express wound healing fusion protein at high levels, and subsequently administered to a wound site (typically re-administered), thereby effecting long term expression of the wound healing fusion protein, which is particularly preferred in skin regeneration (e.g., in treating severe burns).

As previously mentioned, the fusion protein, polynucleotide, vector, together or separately can be co-administered with any suitable factor, preferably a factor which promotes angiogenesis, wound healing, bone growth, related biological activity, or enhances the activity of the fusion protein, polynucleotide or vector. Thus, in some situations, combinations of fusion protein, polynucleotide, or vector and another factor (e.g., bone growth promoting, angiogenic, or wound healing promoting protein), or co-administration of the vector and fusion protein can be desirable. Such co-administration can facilitate systemic treatment of diseases. For example, in the context of angiogenesis-related disorders, such as vascular ischemia, the administration of the fusion protein, fusion protein-encoding polynucleotide, or vector comprising such a polynucleotide can be associated with the administration of a smooth muscle tension modifier (e.g., a vasodilator, such as a direct vasodilator (e.g., hydralazine, minoxidil, reserpine, or combinations thereof), an atrial natriuretic peptide, a vasoactive intestinal peptide, a histamine, an epinephrine or modified epinephrine (e.g., a β-2 receptor targeted epinephrine homolog or a naturally occurring epinephrine administered in a β-2 receptor-targeting manner), a bradykinin, a paracrine which induces vasodilatation (e.g., adenosine, carbon dioxide, hydrogen ion, nitric oxide, or an endothelin), an ACE inhibitor (e.g., an ACE2 inhibitor), an adrenergic receptor blocker, a vascular-associated parasympathetic nervous system stimulator (e.g., acetylcholine), an angiotensin II-receptor blocker (ARB—e.g., tasosartan), and/or a calcium channel blocker). Other suitable non-vasodilator compounds which lower vascular resistance can be administered, and/or the application of mechanical techniques for lowering resistance (and, thus, increasing blood flow) can be applied, near or at tissues associated with the administration of the angiogenic fusion protein, fusion protein-encoding polynucleotide, or vector, and/or at one or more distal/peripheral tissues. Additionally, one or more biologically active catecholamines can be co-administered in association with the fusion protein, polynucleotide, or vector, particularly in association with the administration of an angiogenic fusion protein, polynucleotide, or vector to or near the heart. When an angiogenic fusion protein, polynucleotide, or vector is administered as a prophylactic (e.g., to a tissue at risk of ischemia due to an imminent vascular occlusion), co-administration of a factor which reduces the risk of occlusion, e.g., an anti-coagulant (such as a heparin, antithrombin III, a plasminogen, a prostacyclin (e.g. prostaglandin I or PGI₂), Protein C, tissue plasminogen activator (t-PA), the anti-coagulants described in U.S. Pat. No. 6,121,435, or homologs thereof), or an LDL cholesterol reducing factor (e.g., a bile acid sequestrant, such as cholestyramine, colestipol, and nicotinic acid (niacin), a statin (HMG CoA reductase inhibitor), such as, lovastatin, pravastatin, simvastatin, and atorvastatin (Lipitor), rosuvastatin calcium (Crestor), an endothelin agonist (e.g., tezosentan), a gemfibrozil, a probucol, or a clofibrate) also is contemplated. Administration of the fusion protein, polynucleotide, or vector can be in conjunction with a surgical method where an occlusion is removed, or where lipids (e.g., LDL cholesterol) are removed from cells which then are re-administered (i.e., an autotransplant).

In certain situations, it can be desirable to co-administer a factor which induces or promotes hematopoiesis with the fusion protein, polynucleotide, or vector of the invention. Any suitable hematopoietic factor can be co-administered in any suitable form. The hematopoietic factor can be any suitable type of hematopoietic factor. Examples of such factors include red blood cell growth promoting factors (e.g., erythropoietin (EPO)), megakaryocyte growth promoting factors (e.g., granulocyte-macrophage colony stimulating factor (GM-CSF)), eosinophil growth promoting factors (e.g., GM-CSF), neutrophil growth promoting factors (e.g., granulocyte colony-stimulating factor (G-CSF)), and monocytes growth promoting factors (e.g., macrophage colony-stimulating factor (M-CSF)). Such factors can be administered in association with an administration of stem cells (or, more particularly haematopoietic precursor cells or angioblasts, such as bone marrow derived angioblasts (as described in, e.g., Kocher et al., Nat. Med., 7(4), 430-36 (2001)), or alternatively, administration of developed cells, such as cardiac myocytes (using techniques described in or similar to those provided in Li et al., J. Mol. Cell Cardiol., 31, 513-22 (1999)). Such cells can be obtained from a heterologous source or from a patient to which they are to be re-administered (e.g., through obtaining such cells from removed (and possibly cultured) bone marrow, blood, or fatty tissues of the individual). Similar co-administration of relevant cells can be performed for wound healing and bone growth promoting aspects of the invention (e.g., co-administration of keratinocytes in wound healing or of osteoblasts for promotion of bone growth). Co-administration of hematopoietic factors is particularly preferred in association with the administration of a wound healing fusion protein, polynucleotide, or vector of the invention.

Factors which block or enhance events in the angiogenic, wound healing, or bone growth promoting pathway also can be administered in association with the fusion protein, polynucleotide, or vector. For example, co-administration of PLC-γ, Ras, Shc, Nck, PKC and/or PI3-kinase can be co-administered with the polynucleotide, vector, or fusion protein, to induce downstream signal pathways associated with VEGFR-2, as can factors which block such downstream interactions. Factors which induce VEGF expression, such as PDGF, keratinocyte growth factor, EGF, TNF-α, IGF-1, thyroid-stimulating hormone, IL-1α, IL-4, IL-6, TGF-β, IL-1β, prostaglandin E2 (PGE₂), ACTH, v-Ha-ras, v-raf, and v-myc, also can be co-administered with the fusion protein, polynucleotide, or vector, as can chemical agents which upregulate VEGF expression, such as phorbol myristate acetate (as described in, e.g., Ilan et al., J. Cell Sci., 111, 3621-31 (1998)) or other phorbol esters. The fusion protein, polynucleotide, or vector can be advantageously administered after or during administration of such a phorbol ester compound, which may induce vascular tube formation in collagenous tissues, as administration of an angiogenic fusion protein may sustain the integrity of the newly formed vascular tube and prevent endothelial cell apoptosis thereafter which might otherwise result from phorbol ester-induced angiogenesis. Factors which upregulate factors corresponding or related to the second peptide portion also or alternatively can be co-administered. For example, progesterone can be co-administered with a fusion protein, polynucleotide, or vector to upregulate HBNF expression. Co-administration of factors that upregulate expression of a desired angiogenic factor, bone growth promoting factor, or wound healing promoting factor, where such a factor does not correspond or related to a peptide portion of the fusion protein also is within the scope of the invention (e.g., administration of a factor which upregulates Ang-1 expression in conjunction with the administration of a VEGF₁₂₁/HBNF fusion protein).

Factors that inhibit inflammation also or alternatively can be administered with the fusion protein, polynucleotide, or vector of the invention. The inflammation inhibitor can be any suitable inflammation inhibitor. Examples of suitable inflammation inhibitors are provided in, e.g., U.S. Pat. No. 5,830,880. In some circumstances, co-administration of a suitable factor which inhibit thrombosis can be desirable, such as the factors described in U.S. Pat. No. 5,955,576.

Factors which are co-administered with the fusion protein, polynucleotide, or vector of the invention, can be co-administered in any suitable manner, and in any suitable order (i.e., concurrently or sequentially), such as administering a fusion protein, polynucleotide, or vector of the invention and separately administering a vector containing a polynucleotide encoding such a factor (or homolog thereof), or administering a vector containing a polynucleotide encoding such a factor which also encodes a fusion protein of the invention.

Factors which reduce naturally occurring anti-angiogenic factors (e.g., an endostatin (or fragment thereof, such as the collagen XVIII fragment), angiotensin (or fragment thereof, such as the plasminogen fragment), thrombospondins (e.g., thrombospondin-1), the 16 kDa fragment of prolactin, and vasostatin (or calreticulin)), Cartilage-derived inhibitor (CDI), CD59 complement fragment, Gro-beta, Heparinases, Heparin hexasaccharide fragment, Human chorionic gonadotropin (hCG), IFNs, Interferon inducible protein (IP-10), IL-12, Kringle 5 (plasminogen fragment), 2-Methoxyestradiol, Placental ribonuclease inhibitor, Plasminogen activator inhibitor, Platelet factor-4 (PF4), Proliferin-related protein (PRP), Retinoids, Tetrahydrocortisol-S, other anti-angiogenic C-X-C chemokines, and/or vasculostatin also can be suitable for co-administration with the fusion protein, polynucleotide, or vector. For example, one or more factors which block one or more anti-angiogenic factors from binding with receptors required for activation, or which prevent cleavage or other conformational changes required for immature anti-angiogenic proteins to develop anti-angiogenic activity (e.g., blocking cleavage required for development of a mature, anti-angiogenic, endostatin, or preventing conversion of plasminogen to angiostatin), can be administered with the angiogenic fusion protein, polynucleotide, or vector. Such factors can be administered in any suitable form (e.g., as a polynucleotide inserted into a separate vector or the same vector with a fusion protein-encoding polynucleotide). Alternatively, one or more antisense polynucleotides which prevent transcription and/or translation of an anti-angiogenic gene, or one or more monoclonal antibodies which deactivate the anti-angiogenic factor or block its activity.

Administration of anti-angiogenic factors or angiogenic factor antagonists in association with the administration of angiogenic fusion proteins, polynucleotides, and/or vectors can be desirable in some conditions. For example, administration of such factors can provide control over the level of blood vessel growth to be achieved by administration of the fusion protein, polynucleotide, or vector, and can provide a method of avoiding undesirable levels of blood vessel growth resulting from administration or expression of the angiogenic fusion protein.

The invention further provides a method comprising co-administration of different fusion proteins of the invention, polynucleotides encoding such various fusion proteins, or vectors containing such polynucleotides. For example, a VEGF₁₂₁/angiopoietin fusion protein can be co-administered with a VEGF₁₂₁/aFGF fusion protein, a VEGF₁₂₁/HBNF fusion protein, or all three fusion proteins can be co-administered.

In addition to the other administration techniques described herein, the vector composition or fusion protein composition can be administered by direct surgical implantation. Alternatively or additionally, the fusion protein and/or vector composition can be co-administered with a group of therapeutic cells, e.g., stem cells, macrophages, or neurophils. For example, an angiogenic vector composition of the invention can be co-administered with stem cells to an ischemic location in the heart. The use of the vector composition and fusion protein of the invention also can be useful in organ generation and organ transfer.

The angiogenic fusion protein and vector compositions of the invention can be used to treat a wide variety of ailments including, e.g., coronary artery disease, peripheral vascular disease, congestive heart failure (e.g., left ventricular dysfunction and left ventricular hypertrophy), neuropathy (peripheral or otherwise), avascular necrosis (e.g., bone or dental necrosis), mesenteric ischemia, impotence (or erectile dysfunction), incontinence, arterio-venous fistula, veno-venous fistula, stroke, cerebrovascular ischemia, muscle wasting, pulmonary hypertension, gastrointestinal ulcers, vasculitis, non-healing ischemic ulcers, retinopathies, restenosis, cancer, orthosclerosis, radiation-induced tissue injury (such as that common with cancer treatment), and other hypoxia-associated or low blood perfusion-associated disorders. In addition, the angiogenic fusion protein and vector compositions also find utility in the study and/or aid of wound healing (e.g., healing of ischemic ulcers), plastic surgery procedures (e.g., healing or reattachment of skin and/or muscle flaps), prosthetic implant healing, vascular graft patency, and transplant longevity. Thus, the invention provides methods of treating such ailments by administration of the fusion protein and/or vector compositions.

Compositions containing the bone growth-promoting fusion protein and, more preferably, the vector containing a polynucleotide encoding such fusion protein can be used to treat diseases like osteoporosis, improve poor bone healing (e.g., fibrous non-union), to promote implant integration and the function of artificial joints, to stimulate healing of other skeletal tissues such as Achilles tendon, or as an adjuvant to repair large defects. Such compositions also can be used to treat implant interface failures and allograft failures. Furthermore, the administration of such compositions provides a method of treating osteogenesis imperfecta (OI) and fractures, as well as facilitating bone reconstruction. The compositions also can be used for the treatment of periodontal tissues. Such compositions can also be used for treatment of rheumatoid and osteo arthritis. The methods and compositions of the invention also can be used for prophylactic purposes, e.g., in closed and open fracture reduction and the improved fixation of artificial joints.

The administration of the wound healing fusion protein and/or vector compositions of the invention can be used to treat ulcers (e.g., decubitus ulcers, venous stasis ulcers, arterial ulcers, diabetic ulcers and stasis ulcers), lesions, injuries, burns, trauma, periodontal conditions, lacerations, and other conditions, promote/enhance spinal chord healing, and promote/enhance tendon and/or ligament healing (either through direct healing or by promoting angiogenesis in such tissues). The fusion protein, polynucleotide, or vector can be used in the treatment of wounds to skin, muscle, neurologic tissue, soft tissue, internal organs, and any other suitable part of the body (e.g., those wounds described elsewhere herein). In addition, intraperitoneal wound tissue such as that resulting from invasive surgery can be treated with such compositions. For example, following the surgical removal of a colon section or other tissue, the surgical plane can be coated with the composition prior to closing the surgical site in order to accelerate internal capillary perfusion and healing. In addition, the rate of localized healing can be increased by the subdermal administration or injection of such compositions. Particular areas where application of the wound healing compositions offer therapeutic promise is in the treatment of the diabetic foot, pressure ulcers, and burns. The compositions also are useful in for treating acne, reducing scar tissue, and in recovery from general and plastic surgery. Moreover, the compositions can be used in treatment of dental tissue (e.g., the gums), for example, in conjunction with oral surgery.

The fusion protein, fusion protein-encoding polynucleotide, and vector of the invention are believed to be useful in several medically related contexts. For example, in surgical contexts, the fusion protein, polynucleotide, and/or vector can be used to treat orthopedic surgery-associated avascular necrosis, treat mesenteric ischemia, provide prophylaxis against ischemia in association with ostomies, treat or provide prophylaxis for thoracic ischemia related spinal chord complications (aneurism repairs), treat sexual dysfunction (e.g., urology-prostprostatectomy associated sexual dysfunction—for example in association with radial prostatectomy), provide smooth muscle tone in tissues (e.g., treat incontinence), prevent radiation-induced vascular necrosis (e.g., prevent tooth loss associated with radiation use in dentistry), promote gum and/or tooth regeneration, create and/or promote veno-venous or arterio-venous anastamosis, and enhance cartilage, tendon, and/or ligament repair replacement (either through direct healing or by promoting angiogenesis in such tissues or tissues associated therewith). The fusion protein, polynucleotide, or vector can be used to provide vascular protection in association, e.g., by inducing nitric oxide production and/or prostacyclin production, inducing antiapoptotic signaling pathways, and/or enhance the antithrombogenic and anti-inflammatory properties of mature endothelium.

The fusion protein, polynucleotide, and vector of the invention also can be useful in neurological applications, such as inducing angiogenesis in the treatment of cerebrovascular-associated vascular obstructive disease, acting as a neurotrophic agent (in association with peripheral neuropathies and/or degenerative disorders), treatment of sonic-related hearing loss, and enhancing CNS drug delivery by modifying the properties of the blood-brain barrier. The fusion protein, polynucleotide, and vector also can be useful in endocrine/metabolic contexts, such as the treatment of muscle wasting (sarcopenia) and the promotion/induction of hair growth (particularly in association with a hedgehog protein second peptide portion). Other cardiovascular-associated uses for the compositions of the invention include reducing oxidative stress, treatment of non-ischemia associated causes of heart failure, enhancing revascularization of vascular grafts (AV shunts, arterial conduits, and endovascular grafts), mobilizing progenitor cells to sites of interest, and improvement of organ transplant outcome. Pulmonary and gastrointestinal applications of the fusion protein, polynucleotide, and vector include administration in association with liver regeneration, treatment of pulmonary hypertension, and providing/increasing blood supply to a transplanted lung. Rheumatological/renal applications of the fusion protein, polynucleotide, and vector include the treatment of vasculitis, modulation of renal permeability and function, modulation of peritoneal permeability and function, and promotion of growth factors delivery to such tissues through such permeability modulation.

The methods of this invention are closely related in function. Thus, it is to be understood that the disclosure with respect to any aspect of the invention can be applied to any other suitable aspect. For example, forms of administration and delivery techniques for fusion protein compositions can be used for polynucleotide or, more particularly, vector compositions, and vice versa. Similarly, references to administration of the fusion protein, polynucleotide, or vector of the invention encompasses the administration of pharmaceutically acceptable containing the fusion protein, polynucleotide, or vector, as applicable.

The invention also provides other related fusion proteins, comprising at least a first and second peptide portion, which exhibit similar biological activity (i.e., promotion of angiogenesis, wound healing, bone growth, or a combination thereof) as the VEGF fusion proteins of the invention. Such fusion proteins can comprise any combination of two or more of the second peptide portions described with respect to the VEGF fusion proteins of the invention that results in a fusion protein which promotes angiogenesis, bone growth, or wound healing. Preferred examples of such “second peptide portion fusion proteins” include fusion proteins that comprise a first HBNF peptide portion, an MK peptide portion, or a SEAP peptide portion, fused to any of the angiogenic, wound healing, or bone growth promoting second peptide portions described herein, including HBNF/SEAP fusion proteins, MK/SEAP fusion proteins, HBNF/CTGF fusion proteins, HBNF/scatter factor fusion proteins, MK/HGF fusion proteins, HBNF/BMP fusion proteins, MK/BMP fusion proteins, HBNF/FGF proteins, SEAP/BMP fusion proteins, SEAP/decorsin fusion proteins (or other second peptide portion fusion proteins wherein at least one of the peptide portions include a heterologous receptor binding domain, preferably an integrin binding domain), HBNF-MK/Ephrin fusion proteins, MK/FGF fusion proteins, HBNF/Ang-1 fusion proteins, MK/Ang-1 fusion proteins, other HBNF-MK/ARF fusion proteins (such as MK/NL5 fusion proteins), Ang-1/SEAP fusion proteins, and other ARF/SEAP fusion proteins (such as a NL1/SEAP fusion proteins). The second peptide portion fusion protein can be modified to reduce immunogenicity in a host as described above with respect to the VEGF fusion proteins of the invention, for example by incorporating a flexible linker between the peptide portions of the fusion protein that results in a lower immunogenicity than is exhibited against a direct fusion of the two peptide portions. Desirably, the second peptide portion fusion protein exhibits multiple biological functions (e.g., promotes at least two distinct aspects of angiogenesis, bone growth, or wound healing). Preferably, such fusion proteins exhibit higher levels of angiogenesis, bone growth, and/or wound healing than a protein consisting essentially of at least one of the fusion protein peptide portions, more preferably than both peptide portions, and most preferably than the co-administration of two proteins that separately consist essentially of the fusion protein's peptide portions. Routine methods for determining whether such combinations produce such desired effects are provided herein, and the second peptide portions described herein are expected, when combined to produce fusion proteins, to promote angiogenesis, bone growth, and/or wound healing when administered or expressed in a mammalian host. The invention further provides polynucleotides encoding such second peptide portion fusion proteins (e.g., a polynucleotide encoding an HBNF/SEAP, HBNF/BMP, HBNF/CTGF, or HBNF/TGF-β fusion protein), and vectors comprising such polynucleotides, which preferably are adenoviral vectors, and more preferably targeted adenoviral vectors, as described herein with respect to the VEGF fusion protein aspects of the invention. The polynucleotide can be any suitable polynucleotide, obtained by and/or modified by the techniques described with respect to VEGF fusion protein-encoding polynucleotides of the invention and the vector can be any of the vectors described herein (e.g., a modified adenoviral vector that results in a lower host immune response upon administration than a wild-type adenoviral vector through the presence of a trans acting factor such as HSV ICP0). The second peptide portion fusion proteins, polynucleotides, and vectors can be used in vector or fusion protein compositions similar to those described herein with respect to the VEGF fusion proteins and related polynucleotides of the invention. The second peptide portion fusion proteins can be co-administered with any of the factors described as potential co-administration partners for the VEGF fusion proteins of the invention (e.g., in association with an administration of angioblasts, stem cells, or other precursor cells, or in association with a vasodilator). The second peptide portion fusion proteins, polynucleotides, or vectors can be administered in the same manner as is described herein with respect to the VEGF fusion proteins of the invention, and can be used to treat any of the specific diseases provided herein with respect to such fusion proteins.

The invention further provides non-fusion protein proteins corresponding to the modified VEGF portions, modified second peptide portions, and second peptide fragments of the invention. Preferred examples of such proteins include the above-described HBNF homologs and fragments, MK homologs and fragments, SEAP homologs and fragments, proteins corresponding to any of the VEGFs or second peptide portions of the invention which comprise a heterologous receptor binding domain, and proteins containing RGD domains (e.g., a decorsin-related protein). Polynucleotides encoding such factors can be obtained or produced using the techniques described herein. Such polynucleotides can be contained in any of the above-described vectors of the invention, preferably in one of the adenoviral vectors of the invention. The invention further provides a method of promoting angiogenesis, bone growth, and/or wound healing comprising administering such proteins, polynucleotides, or vectors. Such proteins can be co-administered with any of the factors described above that are suitable for co-administration with the VEGF fusion proteins of the invention. Such proteins, polynucleotides, and vectors can be administered to treat any of the diseases discussed herein with respect to the VEGF fusion proteins of the invention.

The invention also provides a modified VEGF, which has at least one domain that allows the modified VEGF to exhibit greater heparin binding than its wild type counterpart. Examples of such a VEGF include VEGF_{121.2}, VEGF_{121.3}, VEGF_{121.5} and VEGF_{121.6}, described above, which exhibit higher levels of heparin binding than VEGF₁₂₁. Polynucleotides encoding such VEGFs can be obtained using the techniques described herein, and such polynucleotides can be inserted into any of the aforementioned vectors. Such VEGFs can be administered to promote angiogenesis, bone growth, or wound healing, using the methods described herein with respect to the VEGF fusion proteins, polynucleotides, and vectors of the invention. For example, such modified VEGFs can be administered in association with a vasodilator, or angioblasts, and such modified VEGFs can promote wound healing in association with a suitable wound healing factor, such as a SEAP, CTGF, HBNF, PDGF, or TGF-β.

Any methods of administration described above with respect to the VEGF fusion proteins, polynucleotides, and vectors of the invention can be applied to a protein comprising or consisting of any of the above-described VEGFs, including the heparin-binding VEGFs, to promote angiogenesis, bone growth, and/or wound healing, or to treat or prevent any of the diseases discussed herein. For example, such VEGFs (e.g., VEGF₁₂₁, VEGF₁₆₅, VEGF₁₄₅, or VEGF₁₈₉), or polynucleotides encoding such VEGFs, or related vectors, can be administered to treat ulcers, bone fracture, bone disease, hair loss, or erectility dysfunction, or to promote blood brain barrier permeability or vascular regularity after inducing angiogenesis with another angiogenic agent. Moreover, such VEGFs can be co-administered with any of the agents described above as potential co-administration partners with respect to the VEGF fusion proteins of the invention (e.g., a vasodilator or a culture of angioblasts).

### EXAMPLES

The following examples further illustrate the present invention but should not be construed as in any way limiting its scope.

### Example 1

This example describes the generation of a polynucleotide encoding a VEGF₁₂₁/Ang-1 fusion protein, the production of a vector containing such a polynucleotide, and the administration of such a vector to induce angiogenesis in a mammalian host.

The oligonucleotide primers CGCGGATCCACCATGAACTTTCTGCTGTCTT GG (SEQ ID NO: 69) (VEGF₁₂₁ primer 1) and CTAAATGGTTTCTCTTCCTCCCCGCCT CGGCTTGTCACA (SEQ ID NO: 70) are used to amplify an PCR product comprising the VEGF₁₂₁ gene sequence from plasmid pUCVEGF₁₂₁ or similar plasmid (e.g., one of the pMT-VEGF plasmids described in U.S. Pat. No. 5,219,739), using standard PCR techniques. Primers TGTGACAAGCCTGAGGCGGGAGGAAGAGAAACCATTTAG (SEQ ID NO: 71) and CGCGGATCCTCAAAAATCTAAAGGTCGA (SEQ ID NO: 72) (Ang-1 primer 1) are used to amplify a PCR product comprising a fragment of the human Ang-1 gene corresponding to the sequence encoding amino acid residues 275-498 of Ang-1 from plasmid pAd3511CMVAng1. Aliquots of the amplified VEGF₁₂₁ and Ang-1 fragment PCR products are mixed. VEGF₁₂₁ primer 1 and Ang-1 primer 1 are used in another round of PCR using standard techniques utilizing the mixed aliquots as a template material, to form a resulting PCR product, comprising a polynucleotide sequence (SEQ ID NO: 73), encoding a VEGF₁₂₁/Ang-1 fusion protein (SEQ ID NO: 74), which comprises the VEGF-A signal sequence.

The VEGF₁₂₁/Ang-1-encoding PCR product is cut with Bam HI and cloned into a pAd3511CMV transfer vector, which comprises nucleotides 1-4511 of the adenoviral serotype 5 genome, except nucleotides 353-3511 (which encompass the adenoviral E1A and E1B coding regions), the CMV promoter, a multiple cloning site (including Bam HI), the SV40 poly A site, and a splice donor/acceptor site between Ad5 nucleotides 353 and 3511.

After insertion of the Bam HI fragment, the recombinant transfer vector is used to generate a transfection plasmid capable of producing an E1-deleted adenoviral vector containing the VEGF₁₂₁/Ang-1 fusion protein-encoding sequence positioned in the E1 deletion upon transfection into a suitable host cell. The transfection plasmid can be generated by any suitable technique. Examples of such techniques include homologous recombination, or ligation to, one or more additional plasmids comprising the remainder of the adenoviral genome except the desired deleted regions (i.e., E1, E3, and optionally other regions, e.g., the E4 region). Any suitable homologous recombination technique can be used to generate the vector-producing plasmid. Examples of such techniques are provided in, e.g., Chinnadurai et al., J. Virol., 32, 623-28 (1979), Berkner et al., Biotechniques, 6, 616-28 (1998), Chartier et al., J. Virol., 70, 4805-10 (1996), and International Patent Application WO 96/25506. A preferred homologous recombination technique is described in International Patent Application WO 99/15686. Alternatively, any suitable ligation technique can be used, such as the techniques described in, e.g., Stow, J. Virol., 37(1), 171-80 (1981), Stow, Nucl. Acids Res., 10(17), 5105-19 (1982), and Berkner et al., Nucl. Acids Res., 11(17), 6003-20 (1983).

After a suitable transfection plasmid containing the VEGF₁₂₁/Ang-1 fusion protein-encoding sequence is generated, the transfection plasmid is transfected into a suitable E1 complementing cell line, such as a 293-ORF6 cell line (described in International Patent Application WO 95/34671), using standard techniques (e.g., calcium phosphate precipitated transfection), thereby resulting in the production of a stock of E1-deleted, replication-deficient, adenoviral vectors (AdVEGF₁₂₁/Ang-1). Preferably, the vector-cell line system selected is such that replication competent adenovirus (RCA) levels in the stock are confirmed to be less than about 1×10⁷ plaque forming units (pfu), preferably by using the techniques described in U.S. Pat. No. 5,994,106. Levels of viral pfu can be determined using standard techniques (such as the techniques described in Chinnadurai et al., supra and Precious et al., “Purification and Titration of Adenoviruses” in Virology: A Practical Approach, 193-205 (Mahay et al., Eds., IRL Press 1985).

The AdVEGF₁₂₁/Ang-1 vector is administered by needle injection in an appropriate carrier to at least one target location in a mammalian host. Resultant VEGF₁₂₁/Ang-1 fusion gene expression is confirmed by mRNA expression analysis, subsequent administration of an anti-VEGF antibody to the site of vector administration after sufficient time for fusion protein expression, and/or observation of the angiogenic effects of administering the vector, for example, by using the mouse ear or rat hind limb models for testing the angiogenesis-inducing capacity of a molecule, as described in more detail here.

In the mouse ear model, 10⁹-10¹⁰ particles units (pu) of the vector is administered to Apo E^{-/-} mice. All injections are delivered subcutaneously at the base of the ears of anesthetized mice (12 mg/kg xylazine and 60 mg/kg ketamine, IP). Gross morphological changes to the target tissue are observed at various days post-injection. Serial laser Doppler perfusion measurements are taken at various time points post-injection. Changes in blood vessel number are identified using an Olympus B×40F microscope at 400× to examine harvested ears that are perfusion fixed and embedded in paraffin. Control groups receiving other angiogenic proteins, vectors encoding angiogenic proteins (e.g., a heparin-binding VEGF), or null vectors (i.e., vectors containing a non-angiogenic gene or inert spacer in the deleted E1 region), similarly administered, are used for comparative testing.

It is expected that at about four days post-injection, administration of AdVEGF₁₂₁/Ang-1 and resulting expression of the VEGF₁₂₁/Ang-1 fusion protein will result in the formation of blood vessels in greater number and/or volume than vessels formed in animals receiving administration of a heparin-binding form of VEGF, Ang-1, or vector encoding such factors, and that the new blood vessels will exhibit a greater level of vessel maturation than vessels resulting from administration of VEGF₁₂₁ or a vector encoding VEGF₁₂₁.

In the rat hind limb model, AdVEGF₁₂₁/Ang-1 is administered to immature (e.g., six month old) CD rats. The right femoral artery of each rat is removed about seven days before administration of the nucleic acids. Each rat is administered 10⁹-10¹⁰ pu of the vector via two injections to the thigh and one injection to the calf of the rat hind limb. Serial laser Doppler perfusion imaging is used to determine blood flow to foot skin. The rats are sacrificed about 28 days post-injection for angiography and histological analysis of skeletal muscle to determine capillary and arterial numbers. Control groups receiving other angiogenic proteins, vectors encoding angiogenic proteins (e.g., a VEGF), or null vectors, similarly administered, are used for comparative testing.

It is expected that at about 14-28 days post-injection, animals receiving AdVEGF₁₂₁/Ang-1 in the hind limb model will exhibit tissue perfusion levels higher than in control groups receiving administration of Ang-1, a heparin-binding form of VEGF, or a vector encoding such factors.

### Example 2

This example describes the generation of a polynucleotide encoding a VEGF₁₂₁/HBNF fusion protein, the production of a vector containing such a polynucleotide, and the administration of such vectors to a mammalian host to induce angiogenesis.

VEGF₁₂₁ primer 1 and the oligonucleotide primer TTTGCACTCCGCGCCAAATTGCCGCCTCGGCTTGTCACA (SEQ ID NO: 75) are used to amplify a PCR product comprising the VEGF₁₂₁ gene (including the VEGF-A signal sequence) from plasmid pUCVEGF₁₂₁ using a standard PCR technique. Oligonucleotide primers TGTGACAAGCCGAGGCGGCAATTTGGCGCGGAGTGCAAA (SEQ ID NO: 76) and CGCGGATCCTTAATCCAGCATCTTCTCC (SEQ ID NO: 77) (HBNF primer 1) are used to amplify a PCR product comprising a fragment of the HBNF gene from plasmid pHHC12 (as described in Kretschmer et al., supra), which encodes residues 62-136 of human HBNF, using the standard PCR technique. Aliquots of the amplified VEGF₁₂₁ gene and HBNF gene amplified products are obtained and mixed. A PCR product comprising the VEGF₁₂₁/HBNF fusion protein-encoding gene sequence (SEQ ID NO: 78) is obtained and amplified by performing PCR on the mixed amplified products using VEGF₁₂₁ primer 1 and HBNF primer 1. The PCR product is cut with Bam HI and cloned into pAd3511CMV, which is either ligated to, or recombined with, a second plasmid containing the additional desired portions of the adenoviral genome as described in Example 1 to form a transfection plasmid, which is subsequently transfected into cells capable of complementing the production of the encoded E1-deleted adenoviral vector (e.g., 293-ORF6 cells) to produce a replication-deficient adenoviral vector containing the VEGF₁₂₁/HBNF fusion gene. The recombinant adenoviral vector is then administered by direct injection into the mouse ear model or rat hind limb model, as described in Example 1, to assess the angiogenesis-inducing capacity of the expressed VEGF₁₂₁/HBNF fusion protein (SEQ ID NO: 79).

### Example 3

This example describes the generation of a polynucleotide encoding a VEGF₁₂₁/MK fusion protein, the production of a vector containing such a polynucleotide, and the administration of such vectors to a mammalian host to induce angiogenesis.

VEGF₁₂₁ primer 1 and oligonucleotide primer TGCAGTCGGCTCCAAA CTCCCGCCTCGGCTTGTCACA (SEQ ID NO: 80) are used to amplify a PCR product comprising the VEGF₁₂₁ gene PCR product from plasmid pUCVEGF₁₂₁ (including the VEGF-A signal sequence) using a standard PCR technique. Primers TGTGACAAGC CGAGGCGGGAGTTTGGAGCCGACTGCA (SEQ ID NO: 81) and CGCGGATCCC TAGTCCTTTCCCTTCCC (SEQ ID NO: 82) (MK primer 1) are used to similarly amplify a PCR product comprising a fragment of the MK gene from plasmid pMKHC4 (as described in Kretchsmer et al., supra), which encodes human MK residues 59-123. Aliquots are taken from the VEGF₁₂₁ and MK PCR products and mixed. VEGF₁₂₁ primer 1 and MK primer 1 are used to obtain and amplify a PCR product comprising a polynucleotide encoding a VEGF₁₂₁ /MK fusion protein from the mixed amplified PCR products (SEQ ID NO: 83). The VEGF₁₂₁/MK fusion protein-encoding PCR product is cut with Bam HI and cloned into pAd3511CMV, which is either ligated to, or recombined with, a second plasmid containing the additional desired portions of the adenoviral genome as described in Example 1 to form a transfection plasmid, which is subsequently transfected into cells capable of complementing the production of the encoded E1-deleted adenoviral vector (e.g., 293-ORF6 cells) to produce a vector containing the VEGF₁₂₁/MK fusion protein-encoding polynucleotide. The adenoviral vector is then administered by direct injection into the mouse ear model or rat hind limb model, as described in Example 1, to assess the angiogenesis-inducing capacity of the expressed VEGF₁₂₁/MK fusion protein (SEQ ID NO: 84).

### Example 4

This example describes generation of a polynucleotide encoding a VEGF₁₂₁/NL1 fusion protein, the production of a vector containing such a polynucleotide, and the expression of the encoded VEGF₁₂₁/NL1 fusion protein.

VEGF₁₂₁ primer 1 and primer CCATGGGCCCGACGGCTTCCGCCTCGGCTT GTCACA (SEQ ID NO: 85) are used to amplify a PCR product comprising the VEGF₁₂₁ gene sequence (including the VEGF-A signal sequence) from plasmid pUCVEGF₁₂₁. Oligonucleotide primers TGTGACAAGCCGAGGCGGAAGCCGTCGGGCCCATGG (SEQ ID NO: 86) and CGCGGATCCTTAGTGGAAGGTGTTGGGG (SEQ ID NO: 87) (NL1 primer 1) are used to amplify a PCR product comprising a fragment of the NL1 gene from plasmid pAd3511CMVNL1, which encodes residues 270-493 of human NL1. Aliquots are taken from the VEGF₁₂₁ and NL1 amplified PCR products and mixed. VEGF₁₂₁ primer 1 and NL1 primer 1 are used to obtain and amplify a PCR product comprising a polynucleotide sequence encoding a VEGF₁₂₁/NL1 fusion protein (SEQ ID NO: 88) from the mixed PCR products. The resulting fusion-protein encoding PCR product is cut with Bam HI and cloned into pAd3511CMV, which is either ligated to, or recombined with, a second plasmid containing the additional desired portions of the adenoviral genome as described in Example 1 to form a transfection plasmid, which is subsequently transfected into cells capable of complementing the production of the encoded E1-deleted adenoviral vector (e.g., 293-CRF6 cells) to produce an adenoviral vector containing the VEGF₁₂₁/NL1 fusion protein-encoding polynucleotide. The recombinant adenoviral vector is administered by direct injection into the mouse ear model or rat hind limb model, as described in Example 1, to determine the angiogenesis-inducing capacity of the expressed VEGF₁₂₁/NL1 fusion protein (SEQ ID NO: 89).

### Example 5

This example describes generation of a polynucleotide encoding a VEGF₁₂₁/NL5 fusion protein, the production of a vector containing such a polynucleotide, and the expression of the encoded VEGF₁₂₁/NL5 fusion protein.

VEGF₁₂₁ primer 1 and primer GAATGGTCCTTCATTGATCCGCCTCGGCTT GTCACA (SEQ ID NO: 90) are used to amplify a PCR product comprising the VEGF₁₂₁ gene sequence (including the VEGF-A signal sequence) from plasmid pUCVEGF₁₂₁. Oligonucleotide primers TGTGACAAGCCGAGGCGGATCAATGAAGGACCATTC (SEQ ID NO: 91) and CGCGGATCCTCAGTCAATAGGCTTGATCA (SEQ ID NO: 92) (NL5 primer 1) are used to amplify a PCR product comprising a fragment of the NL5 gene from plasmid pAd3511CMVNL5, encoding NL5 residues 272-491. Aliquots are taken from the VEGF₁₂₁ and NL5 PCR products and mixed. VEGF₁₂₁ primer 1 and NL5 primer 1 are used to amplify a resulting PCR product comprising a polynucleotide sequence encoding a VEGF₁₂₁/NL5 fusion protein (SEQ ID NO: 93) from the mixed PCR products. The resulting PCR product is cut with Bam HI and cloned into pAd3511CMV, which is either ligated to, or recombined with, a second plasmid containing the additional desired portions of the adenoviral genome as described in Example 1 to form a transfection plasmid, which is subsequently transfected into cells capable of complementing the production of the encoded E1-deleted adenoviral vector (e.g., 293-ORF6 cells) to produce a vector containing the VEGF₁₂₁/NL5 fusion protein-encoding polynucleotide. The adenoviral vector is administered by direct injection into the mouse ear model or rat hind limb model, as described in Example 1, to assess the angiogenesis-inducing capacity of the expressed VEGF₁₂₁/NL5 fusion protein (SEQ ID NO: 94).

### Example 6

This example describes the generation of a novel Angiopoietin-2 homolog (Ang-2X), and the generation of a polynucleotide encoding a fusion protein that includes a VEGF₁₂₁ domain, the fibrinogen-like domain encoded by KIAA0003, and the coiled coil domain (CCD) from Ang-2X.

Ang-2X was derived from the results of a TNBLAST search of the high-through put sequence database for the human genome project for sequences exhibiting significant levels of identity to Ang-1. Hits were identified on BAC clone RP11-16g12 (GenBank accession number AC018398). Nine contigs were identified and assembled by joining (complement 335846 . . . 336136), (complement 265610 . . . 265440), (complement 133812 . . . 133693), (complement 302082 . . . 302315), (complement 52191 . . . 52060), (complement 238562 . . . 238455), (complement 47913 . . . 47746), (complement 141153 . . . 141079), and (complement 18606 . . . 18544) to derive the following polynucleotide sequence:

ATGTGGCAGATTGTTTTCTTTACTCTGAGCTGTGATCTTGTCTTGGC
CGCAGCCTATAACAACTTTCGGAAGAGCATGGACAGCATAGGAAA
GAAGCAATATCAGGTCCAGCATGGGTCCTGCAGCTACACTTTCCTC
CTGCCAGAGATGGACAACTGCCGCTCTTCCTCCAGCCCCTACGTGT
CCAATGCTGTGCAGAGGGACGCGCCGCTCGAATACGATGACTCGG
TGCAGAGGCTGCAAGTGCTGGAGAACATCATGGAAAACAACACTC
AGTGGCTAATGAAGGTAGAGAATATATCCCAGGACAACATGAAGA
AAGAAATGGTAGAGATACAGCAGAATGCAGTACAGAACCAGACGG
CTGTGATGATAGAAATAGGGACAAACCTGTTGAACCAAACAGCGG
AGCAAACGCGGAAGTTAACTGATGTGGAAGCCCAAGTATTAAATC
AGACCACGAGACTTGAACTTCAGCTCTTGGAACACTCCCTCTCGAC
AAACAAATTGGAAAAACAGATTTTGGACCAGACCAGTGAAATAAA
CAAATTGCAAGATAAGAACAGTTTCCTAGAAAAGAAGGTGCTAGC
TATGGAAGACAAGCACATCATCCAACTACAGTCAATAAAAGAAGA
GAAAGATCAGCTACAGGTGTTAGTATCCAAGCAGAATTCCATCATT
GAAGAACTCGAAAAAAAAATAGTGACTGCCACGGTGAATAATTCA
GTTCTTCAGAAGCAGCAACATGATCTCATGGAGACAGTTAATAACT
TACTGACTATGATGTCCACATCAAACGCAGCTAAGGACCCCACTGT
TGCTAAAGAAGAACAAATCAGCTTCAGAGACTGTGCTGAAGTATTC
AAATCAGGACACACCACGAATGGCATCTACACGTTAACATTCCCTA
ATTCTACAGAAGAGATCAAGGCCTACTGTGACATGGAAGCTGGAG
GAGGCGGGTGGACAATTATTCAGCGACGTGAGGATGGCAGCGTTG
CATTTCAGAGGACTTGGAAAGAATATAAAGTGGGATTTGGTAACCT
CTCAGAAAAATATTGGCTGGGAAATGAGTTTGTTTCGCAACTGACT
AATCAGCAACGCTATGTGCTTAAAATACACCTTAAAGACTGGGAA
GGGAATGAGGCTTACTCATTGTATGAACATTTCTATCTCTCAAGTG
AAGAACTCAATTATAGGNNNNNNNNNNNNNNNNNNNGGCAATGA
TTTTAGCACAAGGGATGGAGCCACCGNCANATGTATTTGCAAATGT
TCACAAATGCTAACAGNAGGTNNNNNNNNNNNNNNNNNNNNNNNN
NNNNNNNTACTGGAAAGGCTCAGGCTATTCGCTCAAGGCCACAAC
CATGATGATCCGACCAGCAGATTTC

where N represents any polynucleotide (SEQ ID NO: 95). A polynucleotide having this sequence is generated using any standard polynucleotide synthesis.

The Ang-2X polynucleotide is predicted to encode a polypeptide having the following amino acid sequence:

Met Trp Gln Ile Val Phe Phe Thr Leu Ser Cys Asp Leu Val Leu Ala Ala Ala
Tyr Asn Asn Phe Arg Lys Ser Met Asp Ser Ile Gly Lys Lys Gln Tyr Gln Val
Gln His Gly Ser Cys Ser Tyr Thr Phe Leu Leu Pro Glu Met Asp Asn Cys
Arg Ser Ser Ser Ser Pro Tyr Val Ser Asn Ala Val Gln Arg Asp Ala Pro Leu
Glu Tyr Asp Asp Ser Val Gln Arg Leu Gln Val Leu Glu Asn Ile Met Glu
Asn Asn Thr Gln Trp Leu Met Lys Leu Glu Asn Ile Ser Gln Asp Asn Met
Lys Lys Glu Met Val Glu Ile Gln Gln Asn Ala Val Gln Asn Gln Thr Ala Val
Met Ile Glu Ile Gly Thr Asn Leu Leu Asn Gln Thr Ala Glu Gln Thr Arg Lys
Leu Thr Asp Val Glu Ala Gln Val Ser Asn Ala Thr Thr Arg Leu Glu Leu
Gln Leu Leu Glu His Ser Leu Ser Thr Asn Lys Leu Glu Lys Gln Ile Leu Asp
Gln Thr Ser Glu Ile Asn Lys Leu Gln Asp Lys Asn Ser Phe Leu Glu Lys Lys
Val Leu Ala Met Glu Asp Lys Hls Ile Ile Gln Leu Gln Ser Ile Lys Glu Glu
Lys Asp Gln Leu Gln Val Leu Val Ser Lys Gln Asn Ser Ile Ile Glu Glu Leu
Glu Lys Lys Ile Val Thr Ala Thr Val Asn Asn Ser Val Leu Gln Lys Gln Gln
His Asp Leu Met Glu Thr Val Asn Asn Leu Leu Thr Met Met Ser Thr Ser
Asn Cys Lys Xaa Xaa Xaa Xaa Val Ala Lys Glu Glu Gln Ile Ser Phe Arg
Asp Cys Ala Glu Val Phe Lys Ser Gly His Thr Thr Asn Gly Ile Tyr Thr Leu
Met Trp Gln Ile Val Phe Phe Thr Leu Ser Cys Asp Leu Val Leu Ala Ala Ala
Tyr Asn Asn Phe Arg Lys Ser Met Asp Ser Ile Gly Lys Lys Gln Tyr Gln Val
Gln His Gly Ser Cys Ser Tyr Thr Phe Leu Leu Pro Glu Met Asp Asn Cys
Arg Ser Ser Ser Ser Pro Tyr

(SEQ ID NO: 96), where Xaa represents any amino acid residue. Ang-2X is believed to have angiogenesis modulating activities similar to angiopoietins, particularly to Ang-2.

Appropriate primers to amplify a PCR product comprising a polynucleotide sequence encoding the VEGF₁₂₁ gene from plasmid pUCVEGF₁₂₁, and (separately) amplifying a PCR product comprising a polynucleotide sequence encoding KIAA0003 from an appropriate plasmid (e.g., pAd3511CMVAng1) are selected. Aliquots of the VEGF₁₂₁ and KIAA0003 PCR products are taken and mixed, and VEGF₁₂₁ primer 1 and an appropriate KIAA0003 primer (e.g., Ang-1 primer 1) then are used to obtain and amplify a polynucleotide encoding a KAP/VEGF₁₂₁ fusion protein (SEQ ID NO: 97) from the mixed PCR products. Alternatively, a KIAA0003 polynucleotide can be synthetically produced, and ligated to the a polynucleotide encoding VEGF₁₂₁ (e.g., the above-described VEGF₁₂₁ PCR product) to form a KAP/VEGF₁₂₁ fusion protein-encoding polynucleotide.

Primers suitable for amplification of the polynucleotide sequence encoding the predicted coiled coil domain of Ang-2X (nucleotides 183-543 of the Ang-2X polynucleotide sequence (SEQ ID NO: 98) encoding predicted amino acid residues 61-181 of Ang-2X (SEQ ID NO: 99)) are selected and used to amplify an Ang-2X CCD-encoding sequence from the synthesized Ang-2X-eneoding polynucleotide (alternatively the sequence is synthesized using standard techniques). Aliquots of the Ang-2X CCD PCR product and KIAA0003/VEGF₁₂₁ PCR products (or ligation products) are mixed to form a template to which Ang-2 CCD and KIAA0003/VEGF₁₂₁ primers are added to obtain and amplify a polynucleotide encoding an Ang-2X CCD/KIAA0003/VEGF₁₂₁ fusion protein. Alternatively, the Ang-2X CCD-encoding polynucleotide is directly fused to the KIAA0003/VEGF₁₂₁ PCR product or ligation product.

The fusion protein-encoding polynucleotide is placed in pAd3511CMV, which is either ligated to, or recombined with, a second plasmid containing the additional desired portions of the adenoviral genome as described in Example 1 to form a transfection plasmid, which is subsequently transfected into cells capable of complementing the production of the encoded E1-deleted adenoviral vector (e.g., 293-ORF6 cells), thereby producing an E1-deleted adenoviral vector encoding the Ang-2X CCD/KIAA0003/VEGF₁₂₁ fusion protein (SEQ ID NO: 100). The recombinant adenoviral vector is then administered to a mammalian host, for example, using one of the models described in Example 1, to assess the angiogenesis-inducing capacity of the novel fusion protein.

### Example 7

This example describes the generation of additional VEGF/Angiopoietin-related factor (ARF) fusion proteins. Suitable primers for obtaining and amplifying a polynucleotide encoding VEGF₁₂₁ (including the VEGF-A signal sequence) from plasmid pUCVEGF₁₂₁ are selected and used to produce a VEGF₁₂₁-encoding polynucleotide PCR product. Primers for obtaining and amplifying a polynucleotide sequence encoding the fibrinogen-like domain (FLD) of NL1 (SEQ ID NO: 101) from plasmid pAd3511CMVNL1, or a polynucleotide sequence encoding the FLD of NL5 (SEQ ID NO: 102) from plasmid pAd3511CMVNL5, are selected and used to produce a NL1 FLD-encoding or NL5 FLD-encoding polynucleotide PCR product, as desired. Aliquots of the VEGF₁₂₁-encoding polynucleotide PCR product and the NL1 FLD-encoding or NL5 FLD-encoding PCR products are obtained and mixed. Suitable primers are selected for obtaining and amplifying a polynucleotide encoding a VEGF121/NL1 FLD fusion protein or VEGF₁₂₁/NL5 FLD fusion protein, as applicable. The fusion protein-encoding polynucleotide is cut with a suitable restriction enzyme and cloned into plasmid pAd3511CMV, which is either ligated to, or recombined with, a second plasmid containing the additional desired portions of the adenoviral genome as described in Example 1 to form a transfection plasmid, which is subsequently transfected into cells capable of complementing the production of the encoded E1-deleted adenoviral vector (e.g., 293-ORF6 cells), thereby producing an E1-deleted adenoviral vector encoding the novel fusion protein. The vector is administered to a mammalian host, for example within the mouse ear or rat hind limb test models described in Example 1, resulting in the production of a VEGF121/NL1 FLD fusion protein (SEQ ID NO: 103) or VEGF₁₂₁/NL5 FLD fusion protein (SEQ ID NO: 104).

### Example 8

This example describes the generation of additional alternative VEGF₁₂₁/Angiopoietin homolog fusion proteins. A polynucleotide encoding a KIAA0003/VEGF₁₂₁ fusion protein is obtained as discussed in Example 6 and placed into pAd3511CMV. Suitable primers are selected for amplifying the KIAA0003/VEGF₁₂₁ fusion protein-encoding polynucleotide from the plasmid. A second set of primers are selected for obtaining and amplifying a polynucleotide sequence encoding the coiled coil domain (CCD), predicted coiled coil domain, or structurally similar domain (e.g., a domain comprising multiple alpha helixes) of an angiopoietin-related factor (ARF).

Predicted coiled coil domain sequences may vary depending on the method used to predict the coiled coil domain. Accordingly, multiple CCD sequences can be provided for a single ARF. Combinations of such sequences or portions thereof also can be used in the context of the invention, and, more specifically, in the context of this Example.

Examples of suitable ARF CCD-encoding polynucleotide sequences include sequences encoding the Ang-1 predicted CCD (e.g., SEQ ID NO: 18 or SEQ ID NO: 105), an Ang-2 predicted CCD (e.g., SEQ ID NO: 106, SEQ ID NO: 107, or SEQ ID NO: 108), the Zapol predicted CCD (SEQ ID NO: 109), a NL5 predicted CCD and/or the predicted CCD of the “Ang-3” of International Patent Application 00/11164 (SEQ ID NO: 110 or SEQ ID NO: 111), a NL1 predicted CCD (SEQ ID NO: 112 or SEQ ID NO: 113), an Ang-3 predicted CCD (SEQ ID NO: 114), an Ang-4 predicted CCD (SEQ ID NO: 115), or a polynucleotide corresponding to the polynucleotides associated with GenBank Accession numbers T11442 (SEQ ID NO: 116) or M62290 (SEQ ID NO: 117). Aliquots of the KIAA0003/VEGF₁₂₁ PCR product and the selected ARF coiled coil domain-encoding polynucleotide PCR product are obtained and mixed. Suitable primers are selected to obtain and amplify a polynucleotide sequence encoding the ARF CCD/KIAA0003/VEGF₁₂₁ fusion protein. Alternatively, direct ligation or synthesis techniques can be used to generate polynucleotides encoding the desired fusion protein. The ARF CCD/KAP/VEGF₁₂₁ fusion protein-encoding polynucleotide is placed into plasmid pAd3511CMV, which is either ligated to, or recombined with, a second plasmid containing the additional desired portions of the adenoviral genome as described in Example 1 to form a transfection plasmid, which is subsequently transfected into cells capable of complementing the production of the encoded E1-deleted adenoviral vector (e.g., 293-ORF6 cells), thereby producing an E1-deleted adenoviral vector encoding the novel fusion protein. The recombinant adenovirus vector is administered to a mammalian host, for example, using one or both of the experimental models described in Example 1, to assess the angiogenesis-inducing capacity of the fusion protein.

### Example 9

This example describes the generation of a polynucleotide encoding another alternative VEGF₁₂₁/Angiopoietin homolog fusion protein.

A polynucleotide corresponding to GenBank Accession No. W77823 (SEQ ID NO: 118) is obtained and cleaved by appropriate endonuclease (e.g., time limited Bal I digestion) or exonuclease, or subjected to PCR with appropriate primers, to obtain a polynucleotide having the sequence:

TATAAGCTGCGGCTGGGGCGATACCATGGCAATGCGGGTGACTCC
TTTACATGGCACAACGGCAAGCAGTTCACCACCCTGGACAGAGAT
CATGATGTCTACACAGGAAACTGTGCCCACTACCAGAAGGGAGG
CTGGTGGTATAACGCCTGTGCCCACTCCAACCTCAACCG

(SEQ ID NO: 119), which corresponds to nucleotides 2-173 of W77823 (alternatively, such a sequence is synthesized using standard techniques). A polynucleotide sequence comprising this sequence fused to the sequence corresponding to GenBank Accession No. T11442 is obtained by additional PCR reactions, blunt ended ligation, or synthetic polynucleotide production. The resulting polynucleotide has the following sequence:

GCCCATGGAGAGACTGCCTGCAGGCCCTGGAGGATGGCCACGAC
ACCAGCTCCATCTACCTGGTGAAGCCGGAGAACACCAACCGCCTC
ATGCAGGTGTGGTGCGACCAGAGACACGACCCCGGGGGCTGGAC
CGTCATCCAGAGACGCCTGGATGGCTCTGTTAACTTCTTCAGGAA
CTGGGAGACGTACAAGCAAGGGTTTGGGAACATTGACGGCGAAT
ACTGGCTGGGCCTGGAGAACATTTACTGGCTGACGAACCAAGGCA
ACTACAAACTCCTGGTGACCATGGAGGACTGGTCCGGCCGCAAAG
TCTTTGCAGAATACGCCAGTTTCCGCCTGGAACCTGAGAGCGAGT
ATTATAAGCTGCGGCTGGGGCGCTACCATGGCAATGCGGGTGACT
CCTTTACATGGCACAACGGCAAGCAGTTCACCACCCAGGACAGAG
ATCATGATGTCTACACAGTATAAGCTGCGGCTGGGGCGATACCAT
GGCAATGCGGGTGACTCCTTTACATGGCACAACGGCAAGCAGTTC
ACCACCCTGGACAGAGATCATGATGTCTACACAGGAAACTGTGCC
CACTACCAGAAGGGAGGCTGGTGGTATAACGCCTGTGCCCACTCC
AACCTCAACCG (SEQ ID NO:120).

This polynucleotide is fused to the KIAA0003/VEGF₁₂₁-encoding polynucleotide sequence described in Example 6, and the fused polynucleotide product inserted into plasmid pAd3511CMV, which is either ligated to, or recombined with, a second plasmid containing the additional desired portions of the adenoviral genome as described in Example 1 to form a transfection plasmid, which is subsequently transfected into cells capable of complementing the production of the encoded E1-deleted adenoviral vector (e.g., 293-ORF6 cells), to produce recombinant E1-deleted adenoviral vectors containing the polynucleotide encoding the W77823/T11442/KIAA0003/VEGF₁₂₁ fusion protein (SEQ ID NO: 121). The recombinant vector is administered to a mammalian host, for example, using one of the models described in Example 1, to assess the angiogenesis-inducing capacity of the novel fusion protein.

### Example 10

This example describes the generation of a VEGF₁₂₁/Angiopoietin homolog fusion protein, comprising a chimeric ARF peptide portion.

A polynucleotide encoding residues 1-280 of Ang2X (SEQ ID NO: 122) is synthesized using standard techniques and fused to a polynucleotide encoding residues 278-498 of Ang-1 (SEQ ID NO: 123), to obtain a polynucleotide (e.g., SEQ ID NO: 124), which encodes an Ang2X/Ang-1 (SEQ ID NO: 125). This polynucleotide is subsequently fused to a polynucleotide encoding VEGF₁₂₁ (including the VEGF-A signal sequence) to produce a VEGF121/Ang2X/Ang-1 chimera-encoding polynucleotide is inserted into an appropriate vector for administration to a mammalian host, preferably to an ischemic tissue, which is predicted to result in a VEGF121/Ang2X/Ang-1 fusion protein (SEQ ID NO: 126), having angiogenesis-modulating properties.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Terms such as “including,” “having,” “comprising,” “containing,” and the like are to be construed as open-ended terms (i.e., meaning “including, but not limited to”) unless otherwise indicated, and as encompassing the phrases “consisting of” and “consisting essentially of.” Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of the preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A fusion protein comprising a first non-heparin-binding VEGF peptide portion and a second non-VEGF peptide portion covalently associated with the first peptide portion, which first and second peptide portions separately promote angiogenesis or bone growth.

2. The fusion protein of claim 1, wherein the first peptide portion comprises a VEGF peptide portion which exhibits a higher affinity for KDR receptors than the flt/flk receptors.

3. The fusion protein of claim 2, wherein the VEGF peptide portion exhibits about equal of less affinity for neurophilin-1, neurophilin-2, or both, as VEGF₁₂₁.

4. The fusion protein of claim 3, wherein the first peptide portion comprises a wild-type VEGF-A amino acid sequence of about 150 amino acid residues or less.

5. The fusion protein of claim 4, wherein the first peptide portion comprises VEGF₁₂₁.

6. The fusion protein of claim 1, wherein the fusion protein has a half-life in a mammalian host at least twice as long as the half-life of a protein consisting essentially of either the first peptide portion, the second peptide portion, or both.

7. The fusion protein of claim 6, wherein the fusion protein has a half-life of at least about 10 minutes in a mammalian host.

8. The fusion protein of claim 7, wherein the second peptide portion comprises a peptide lacking its native multimerization domain or a peptide comprising a non-functional multimerization domain.

9. The fusion protein of claim 1, wherein the fusion protein is more angiogenic than a protein consisting essentially of either the first peptide portion, the second peptide portion, or both.

10. The fusion protein of claim 9, wherein blood vessels resulting from administration of the fusion protein to a mammalian host have less permeability than blood vessels resulting from administration of a protein consisting essentially of the first peptide portion.

11. The fusion protein of claim 9, wherein blood vessels resulting from administration of the fusion protein to a mammalian host exhibit greater maturity than blood vessels resulting from administration of a protein consisting essentially of the first peptide portion.

12. The fusion protein of claim 9, wherein blood vessels resulting from administration of the fusion protein to a mammalian host are associated with more smooth muscle cells, a greater concentration of smooth muscle cells, more endothelial cells, a greater concentration of endothelial cells, or any combination thereof, than blood vessels resulting from administration of a protein consisting essentially of the first peptide portion.

13. The fusion protein of claim 1, wherein the second peptide portion comprises a receptor ligand which is present on a native endothelial cell.

14. The fusion protein of claim 1, wherein the fusion protein diffuses through the extracellular matrix in a mammalian host upon administration to a mammalian host from a point of administration, the cell in which it is expressed, or both, farther than a protein consisting essentially of a naturally occurring heparin-binding form of a VEGF.

15. The fusion protein of claim 14, wherein the fusion protein diffuses through the extracellular matrix in a mammalian host upon administration to a mammalian host from a point of administration, the cell in which it is expressed, or both, farther than a protein consisting essentially of the second peptide portion.

16. The fusion protein of claim 1, wherein administration of the fusion protein to an area in a mammalian host results in greater blood flow in the area of administration than the administration of a protein consisting essentially of the second peptide portion.

17. The fusion protein of claim 1, wherein the second peptide portion comprises a peptide which promotes blood vessel wall maturation, blood vessel wall dilatation, blood vessel remodeling, extracellular matrix degradation, decreases blood vessel permeability, or any combination thereof.

18. The fusion protein of claim 1, wherein the fusion protein is free of any immunoglobulin domains.

19. The fusion protein of claim 1, wherein the second peptide portion comprises an angiopoietin, a fibroblast growth factor, a member of the HBNF-MK family of growth factors, an alkaline phosphatase, or a fragment thereof which promotes angiogenesis, bone growth, wound healing, or any combination thereof.

20. The fusion protein of claim 19, wherein the second peptide portion comprises a peptide that is about 25% or more homologous to angiopoietin-1.

21. The fusion protein of claim 20, wherein the second peptide portion comprises a domain which exhibits about 35% or more homology to the fibrinogen-like domain of Ang-1.

22. The fusion protein of claim 21, wherein the second peptide portion comprises angiopoietin-1 or an angiogenically functional fragment thereof.

23. The fusion protein of claim 22, wherein the second peptide portion comprises an N-terminal truncated form of angiopoietin-1, and the truncated form comprises about 60% or less of the wild-type angiopoietin-1 amino acid sequence.

24. The fusion protein of claim 23, wherein the second peptide portion lacks the multimerization domain of angiopoietin-1.

25. The fusion protein of claim 24, wherein the fusion protein is free of any immunoglobulin domains.

26. The fusion protein of claim 21, wherein the second peptide portion comprises the peptide encoded by KIAA0003.

27. The fusion protein of claim 19, wherein the second peptide portion comprises an acidic fibroblast growth factor or a fragment thereof which promotes angiogenesis, bone growth, wound healing, or any combination thereof.

28. The fusion protein of claim 19, wherein the second peptide portion comprises a member of the HBNF-MK family of growth factors or a fragment thereof which promotes angiogenesis, bone growth, wound healing, or any combination thereof.

29. The fusion protein of claim 28, wherein the second peptide portion comprises a peptide that is about 30% or more homologous to HBNF or MK.

30. The fusion protein of claim 29, wherein the second peptide portion comprises HBNF or MK, or a fragment thereof which promotes angiogenesis, bone growth, wound healing, or combination thereof.

31. The fusion protein of claim 30, wherein the second peptide portion comprises an N-terminal truncated form of HBNF or MK, and the truncated form comprises about 60% or less of the wild-type HBNF or MK amino acid sequence.

32. The fusion protein of claim 1, wherein:
(a) the amino acid sequence of the first peptide portion or second peptide portion, within about 15 amino acids of the fusion point of the fusion protein, lacks an amino acid residue corresponding to an amino acid residue in its wild-type counterpart, or
(b) the fusion protein further comprises a linker positioned between the first peptide portion and second peptide portion.

33. A polynucleotide comprising a nucleotide sequence which, when expressed in a cell permissive for expression of the nucleotide sequence, results in the production of a fusion protein according to claim 1.

34. A vector comprising the polynucleotide of claim 33.

35. The vector of claim 34, wherein the vector is a replication deficient adenoviral vector.

36. The vector of claim 35, wherein the replication deficient adenoviral vector comprises or expresses a modified adenoviral protein, non-adenoviral protein, or both, which increases the efficiency that the vector infects cells as compared to wild-type adenovirus, allows the vector to infect cells which are not normally infected by wild-type adenovirus, results in a reduced host immune response in a mammalian host as compared to wild-type adenovirus, or any combination thereof.

37. The vector of claim 36, wherein the polynucleotide comprises a nucleotide sequence which upon expression results in a fusion protein comprising VEGF₁₂₁ fused to (a) angiopoietin-1, (b) an acidic fibroblast growth factor, (c) a HBNF, (d) a MK, (e) an alkaline phosphatase, or (f) a fragment of any of (a)-(e) which promotes angiogenesis, bone growth, or wound healing.

38. The vector of claim 37, wherein the polynucleotide comprises a second nucleotide sequence that, when expressed, produces a second protein which promotes angiogenesis, bone growth, wound healing, or any combination thereof, and
wherein the nucleotide sequence which results in the production of the fusion protein is operably linked to a first promoter and the second nucleotide sequence is operably linked to a second promoter, such that the initiation of expression of the first nucleotide sequence and the second nucleotide sequence occurs at different times, in response to different factors, or both.

39. A method of promoting angiogenesis, bone growth, wound healing, or any combination thereof in an individual comprising administering to the individual an amount of the fusion protein of claim 1 effective to promote angiogenesis, bone growth, wound healing, or any combination thereof.

40. A method of producing a fusion protein comprising introducing the vector of claim 34 into a cell such that the nucleotide sequence is expressed to produce a fusion protein.

41. A method of producing a fusion protein comprising introducing the vector of claim 35 into a cell such that the polynucleotide is expressed to produce a fusion protein.

42. A method of producing a fusion protein comprising introducing the vector of claim 38 into a cell and permitting or inducing expression of the first nucleotide sequence and the second nucleotide sequence in a manner which imitates a biological cascade associated with angiogenesis, bone growth, or wound healing.

43. A fusion protein comprising a first non-heparin-binding VEGF peptide portion and a second non-VEGF peptide portion covalently associated with the first peptide portion, which first and second peptide portions separately promote angiogenesis, bone growth, wound healing, or any combination thereof, wherein the VEGF peptide portion is at least about 115 amino acids in length or the second peptide portion lacks a collagen binding domain.

44. The fusion protein of claim 43, wherein the second peptide portion promotes angiogenesis.

45. The fusion protein of claim 44, wherein the second peptide portion promotes angiogenesis or bone growth more than wound healing.

46. The fusion protein of claim 45, wherein the fusion protein does not comprise a functional collagen binding domain.
